(19) United States
(12) Patent Application Publication
(10) Pub. No.: US 2026/0242372 A1
(43) Pub. Date: Aug. 20, 2026

(54) NOVEL IMIDAZOPYRIDINE AND PYRAZOLOPYRIDINE SULFONAMIDE DERIVATIVES

(71) Applicant: Hoffmann-La Roche Inc., Little Falls, NJ (US)

(72) Inventors: Guido GALLEY, Rheinfelden (DE); Luca Claudio GOBBI, Buus (CH); Wolfgang GUBA, Muellheim (DE); Dmitry MAZUNIN, Grenzach-Wyhlen (DE); Emmanuel PINARD, Linsdorf (FR); Antonio RICCI, Biel-Benken (CH)

(73) Assignee: Hoffmann-La Roche Inc., Little Falls, NJ (US)

(21) Appl. No.: 18/992,928

(22) PCT Filed: Jul. 18, 2023
(86) PCT No.: PCT/EP2023/069849

§ 371 Date: Jan. 9, 2025

(30) Foreign Application Priority Data
Jul. 20, 2022 (EP) 22185885.5
Aug. 2, 2022 (EP) 22188189.9

(51) Int. Cl. C07D 471/04; A61K 31/437; A61K 31/4439; A61K 31/444; A61K 31/4985; A61K 31/506; A61K 31/519; A61K 31/5365; A61K 31/5383; C07D 487/04; C07D 498/04

(52) U.S. Cl. CPC C07D 471/04; A61K 31/437; A61K 31/4439; A61K 31/444; A61K 31/4985; A61K 31/506; A61K 31/519; A61K 31/5365; A61K 31/5383; C07D 487/04; C07D 498/04

(57) ABSTRACT

The invention relates to novel compounds having the general formula I wherein R¹, R², X₁, X₂, X₃ and W are as described herein, composition including the compounds and methods of using the compounds.

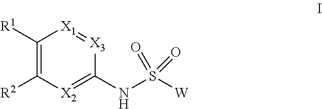

The present invention relates to organic compounds useful for therapy and/or prophylaxis in a mammal, and in particular to compounds that modulate GPR17 activity.

The present invention provides novel compounds of formula I

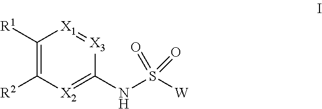

R¹ is alkyl, cyano, cyanoalkyl, cyclopropyl, halo, haloalkoxy, haloalkyl or oxetanyl;
R² is alkoxy, H or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy, H, or halo;
R¹² is H or halo;
R¹³ is alkoxy, H, halo or haloalkoxy;
W is selected from Ring Systems A, B, C, D, or E

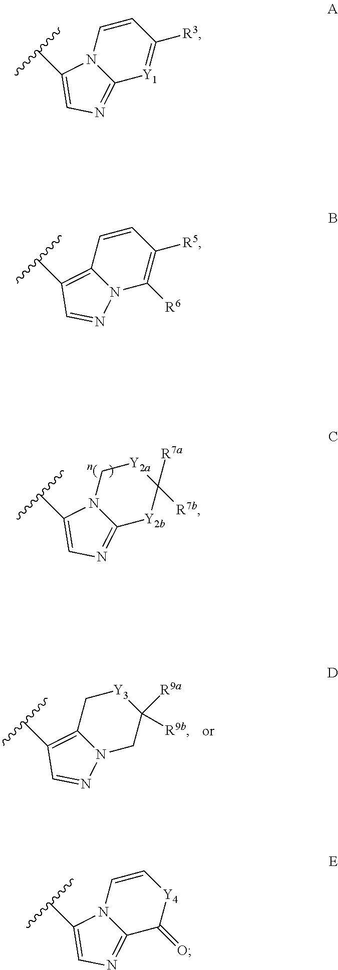

Y₁ is CR⁴ or N;
R³ is alkoxy, alkyl, cyano, cyclopropyl, H, halo, haloalkoxy, haloalkyl, hydroxyalkyl, hydroxyoxetanyl, or oxetanyl;
R⁴ alkoxy, alkyl, cyano, H, or halo;
R⁵ is alkyl, halo, haloalkyl, cyclopropyl or oxetanyl;
R⁶ is H, cyano, alkyl, alkoxy or halo;
Y₂ₐ is CR^{8a} or O and Y_{2b} is CR⁸b or O, wherein only one of Y₂ₐ and Y_{2b} can be O;
n is 0 or 1;
R^{7a} and R^{7b} are independently selected from H, alkyl, alkoxy, or haloalkyl;
R^{8a} and R^{8b} are independently selected from H or alkyl;
Y₃ is O or CH₂;
R^{9a} and R^{9b} are independently selected from H, alkyl, alkoxy, or haloalkyl;
Y₄ is NR¹⁰;
R¹⁰ is alkyl or haloalkyl;
and pharmaceutically acceptable salts thereof.

Furthermore, the invention includes all racemic mixtures, all their corresponding enantiomers and/or optical isomers.

### BACKGROUND OF THE INVENTION

Myelination is a process that occurs robustly during development and despite the abundant presence of oligodendrocyte precursor cells (OPCs) throughout the adult CNS, the transition to myelinating oligodendrocytes and the production of restorative myelin sheaths around denuded axons is impaired in chronic demyelinating diseases. During development, myelination proceeds in a very orderly manner, with OPCs, characterized by expression of markers such as neural/glial antigen 2 (NG2) and platelet-derived growth factor alpha (PDGFRα), differentiating into oligodendrocytes which lose NG2 and PDGFRα expression and gain the expression of markers such as myelin basic protein (MBP) and myelin oligodendrocyte glycoprotein (MOG). The production of myelin by oligodendrocytes is a very tightly regulated process and in the CNS, this can be controlled by interactions with axons, well-understood in the peripheral but not in the central nervous system (Macklin, W. B. (2010). Sci. Signal. 3, pe32-pe32, “The myelin brake: When Enough Is Enough”). Myelination can also be controlled by internal brakes within oligodendrocytes themselves, through the transcription factor EB (TFEB)-PUMA axis or through GPR17 antagonism (Chen, Y., et al. (2009). Nat Neurosci 12, 1398-1406, “The oligodendrocyte-specific G protein-coupled receptor GPR17 is a cell-intrinsic timer of myelination”) (Sun, L. O., et al. (2018). Cell 175, 1811-1826.e21, “Spatiotemporal Control of CNS Myelination by Oligodendrocyte Programmed Cell Death through the TFEB-PUMA Axis”). Myelin serves not only to protect axons and facilitate neuronal transmission, but oligodendrocytes have also been shown to play an important role in metabolism of axons as well as in maintaining the electrolyte balance around axons (Schirmer, L., et al. (2014). Ann Neurol 75, 810-828, “Differential loss of KIR4.1 immunoreactivity in multiple sclerosis lesions”) (Simons, M., and Nave, K.-A. (2015). Cold Spring Harb Perspect Biol. 22, “Oligodendrocytes: Myelination and Axonal Support”).

GPR17 is a Class A orphan G protein-coupled receptor (GPCR). GPCRs are 7 domain transmembrane proteins that couple extracellular ligands with intracellular signaling via their intracellular association with small, heterotrimeric G-protein complexes consisting of G_{α}, G_{β}, G_{γ} subunits. It is the coupling of the GPCR to the Ga subunit that confers results in downstream intracellular signaling pathways. GPR17 is known to be coupled directly to G_{α i/o}, which leads to inhibition of adenylate cyclase activity, resulting in a reduction in cyclic AMP production (CAMP). GPR17 has also been shown to couple to G_{q/11}, that targets phospholipase C. Activation of phospholipase C leads to the cleavage of phosphatidylinositol 4,5-bisphosphate which produces inositol triphosphate (IP₃) and diacylglycerol (DAG). IP₃ consequently binds to the IP₃ receptor on the endoplasmic reticulum and causes an increase in intracellular calcium levels (Hanlon, C. D., and Andrew, D. J. (2015). J Cell Sci. 128, 3533-3542, “Outside-in signaling-a brief review of GPCR signaling with a focus on the Drosophila GPCR family”) (Inoue, A., et al. (2019), Cell 177, 1933-1947.e25, “Illuminating G-Protein-Coupling Selectivity of GPCRs”).

The role of GPR17 in myelination was first identified in a screen of the optic nerves of Olig1 knockout mice to identify genes regulating myelination. GPR17 expression was found to be expressed only in the myelinating cells of the CNS and absent from the Schwann cells, the peripheral nervous system's myelinating cells. The expression of GPR17 was found to be exclusively expressed in the oligodendrocyte lineage cells and was downregulated in myelinating oligodendrocyte (Chen, Y., et al. (2009)). Specifically, GPR17 expression is found to be present at low levels early on in the OPC and increases in the pre-myelinating oligodendrocyte before the expression is downregulated in the mature, myelinating oligodendrocyte (Boda, E., et al. (2011), Glia 59, 1958-1973, “The GPR17 receptor in NG2 expressing cells: Focus on in vivocell maturation and participation in acute trauma and chronic damage”) (Dziedzic, A., et al. (2020). Int. J. Mol. Sci. 21, 1852, “The gpr17 receptor—a promising goal for therapy and a potential marker of the neurodegenerative process in multiple sclerosis”) (Fumagalli, M. et al. (2011), J Biol Chem 286, 10593-10604, “Phenotypic changes, signaling pathway, and functional correlates of GPR17-expressing neural precursor cells during oligodendrocyte differentiation”). GPR17 knockout animals were shown to exhibit precocious myelination throughout the CNS and conversely, transgenic mice overexpressing GPR17 in oligodendrocytes with the CNP-Cre (2′, 3′-cyclic-nucleotide 3′-phosphodiesterase) promoter exhibited myelinogenesis defects, in line with what is to be expected of a cell-intrinsic brake on the myelination process (Chen, Y., et al. (2009)). Furthermore, loss of GPR17 enhances remyelination following demyelination with lysophosphatidylcholine-induced demyelination (Lu, C., Dong, et al. (2018), Sci. Rep. 8, 4502, “G-Protein-Coupled Receptor Gpr17 Regulates Oligodendrocyte Differentiation in Response to Lysolecithin-Induced Demyelination”). As such, antagonism of GPR17 that promotes the differentiation of oligodendrocyte lineage cells into mature, myelinating oligodendrocytes would lead to increase in myelination following demyelination.

Multiple sclerosis (MS) is a chronic neurodegenerative disease that is characterized by the loss of myelin, the protective fatty lipid layer surrounding axons, in the central nervous system (CNS). Prevention of myelin loss or remyelination of denuded axons is thought to prevent axonal degeneration and thus prevent progression of the disease (Franklin, R. J. (2002), Nat Rev Neurosci 3, 705-714, “Why does remyelination fail in multiple sclerosis?”). Due to the restorative impact that myelin repair has on the central nervous system, such a treatment will benefit all types of MS namely relapse-remitting, secondary progressive, primary progressive and progressive relapsing MS. Reparation of lost myelin will alleviate neurological symptoms associated with MS due to the neuroprotective effect of preserving axons.

Due to the essential role that myelination plays in functioning of the nervous system, facilitating OPC to oligodendrocyte differentiation has the potential to impact multiple diseases where white matter defects/irregularities due to either loss of myelinating oligodendrocytes or hampered differentiation of OPCs to oligodendrocytes have been observed, due to the disease itself or inflammation. This is in addition to the diseases where GPR17 expression itself is altered.

The diseases that GPR17 antagonism can be thus used to yield a positive disease outcome include, but are not limited to:

Direct damage to myelin sheaths:
Metabolic conditions that lead to destruction of central myelin such as central pontine myelinolysis, extra-pontine myelinolysis due to overly-rapid correction of hyponatremia in conditions for instance, but not limited to, alcoholism, liver disease, immunosuppression after transplantation
Carbon monoxide poisoning where oligodendrocyte dysfunction and failure to regenerate has been reported in the deep white matter layers of the brain
Nutritional deficiency that results in myelin loss or failure to properly generate myelin during development
Virus-induced demyelination

Primary demyelinating disorders
Multiple Sclerosis (relapse-remitting, secondary progressive, primary progressive and progressive relapsing MS)
Acute and multiphasic disseminated encephalomyelitis
Neuromyelitis optica spectrum disorders including optic neuritis
Transverse myelitis
Leukodystrophies such as adrenoleukodystrophy, adrenomyeloneuropathy and other inherited leukodystrophies that result in myelin loss

CNS disorders with associated myelin loss:
Alzheimer's Disease
Schizophrenia
Parkinson's Disease
Huntington's disease
Amyotrophic lateral
Ischemia due to stroke

Other diseases:
Inflammation in the CNS for instance following encephalitis, primary angiitis, meningitis

The compounds of formula I bind to and modulates GPR17 activity.

The compounds of formula I are therefore particularly useful in the treatment of diseases related to GPR17 antagonism.

The compounds of formula I are particularly useful in the treatment or prophylaxis of multiple sclerosis (MS), conditions related to direct damage to myelin sheaths such as carbon monoxide poisoning or virus induced demyelination, primary demyelinating disorders such as acute and multiphasic disseminated encephalomyelitis, and other CNS disorders associated with myelin loss such as Alzheimer's disease, schizophrenia, Parkinson's disease and Huntington's disease.

### SUMMARY OF THE INVENTION

The present invention provides novel compounds of formula I

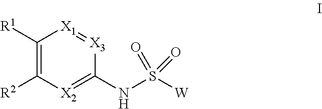

wherein,
R¹ is alkyl, cyano, cyanoalkyl, cyclopropyl, halo, haloalkoxy, haloalkyl or oxetanyl;
R² is alkoxy, H or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy, H, or halo;
R¹² is H or halo;
R¹³ is alkoxy, H, halo or haloalkoxy;
W is selected from Ring Systems A, B, C, D, or E

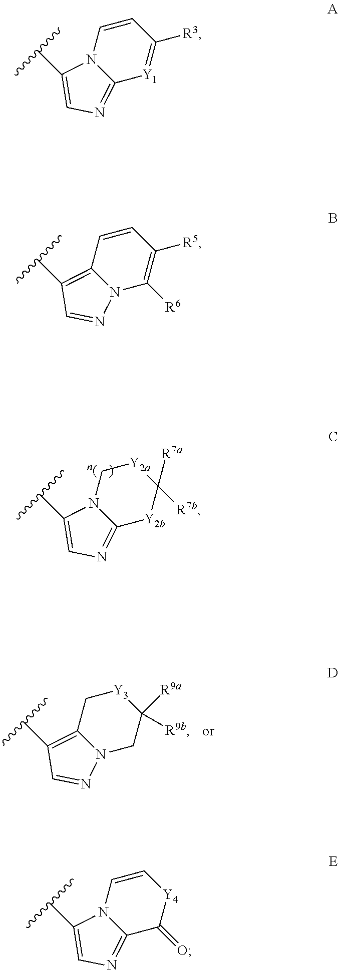

Y₁ is CR⁴ or N;
R³ is alkoxy, alkyl, cyano, cyclopropyl, H, halo, haloalkoxy, haloalkyl, hydroxyalkyl, hydroxyoxetanyl, or oxetanyl;
R⁴ alkoxy, alkyl, cyano, H, or halo;
R⁵ is alkyl, halo, haloalkyl, cyclopropyl or oxetanyl;
R⁶ is H, cyano, alkyl, alkoxy or halo;
Y₂ₐ is CR^{8a} or O and Y_{2b} is CR^{8b} or O, wherein only one of Y₂ₐ and Y_{2b} can be O;
n is 0 or 1;
R^{7a} and R^{7b} are independently selected from H, alkyl, alkoxy, or haloalkyl;
R^{8a} and R^{8b} are independently selected from H or alkyl;
Y₃ is O or CH₂;
R^{9a} and R^{9b} are independently selected from H, alkyl, alkoxy, or haloalkyl;
Y₄ is NR¹⁰;
R¹⁰ is alkyl or haloalkyl;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular examples are methoxy and ethoxy.

The term “cyano” denotes a —C≡N group.

“Cyanoalkyl” means a moiety of the formula-R′—R″, where R′ is alkyl as defined herein and R″ is cyano or nitrile. Particular example is cyanomethyl.

The term “halogen”, “halide” and “halo” are used interchangeably herein and denote fluoro, chloro, bromo or iodo. Particular halogens are fluoro, chloro and bromo.

The term “haloalkyl” denotes a C₁₋₆-alkyl group wherein at least one of the hydrogen atoms of the C₁₋₆-alkyl group has been replaced by the same or different halogen atoms. Particular examples are difluoromethyl, difluoroethyl, difluoropropyl, and trifluoromethyl.

The term “haloalkoxy” denotes a C₁₋₆-alkoxy group wherein at least one of the hydrogen atoms of the C₁₋₆-alkoxy group has been replaced by the same or different halogen atoms. Particular examples are fluoroethoxy, difluoromethoxy and difluoroethoxy.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxymethyl, hydroxyethyl, hydroxypropyl, hydroxymethylpropyl and dihydroxypropyl. Particular example is 1-hydroxy-1-methyl-ethyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol uM.

The abbreviation uL means microliter and is equivalent to the symbol uL.

The abbreviation ug means microgram and is equivalent to the symbol ug.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein Y₂ₐ is CH₂ and Y_{2b} is CR^{8b} or O, wherein R^{8b} is H or alky.

An embodiment of the present invention provides compounds according to formula I as described herein, wherein R^{9a} or R^{9b} are alkyl and the other is H.

An embodiment of the present invention provides compounds according to formula I as described herein, wherein R¹ is alkyl, cyanoalkyl, cyclopropyl, halo, haloalkoxy or haloalkyl.

An embodiment of the present invention provides compounds according to formula I as described herein, wherein R² is alkoxy or halo.

An embodiment of the present invention provides compounds according to formula I as described herein, wherein R¹¹ is alkoxy or H.

An embodiment of the present invention provides compounds according to formula I as described herein, wherein R¹² is H.

An embodiment of the present invention provides compounds according to formula I as described herein, wherein R¹³ is alkoxy.

An embodiment of the present invention provides compounds according to formula I as described herein, wherein W is selected from Ring Systems A, B or C.

An embodiment of the present invention provides compounds according to formula I as described herein, wherein R³ is alkoxy, alkyl, cyclopropyl, halo, haloalkoxy, haloalkyl, hydroxyalkyl or oxetanyl.

An embodiment of the present invention provides compounds according to formula I as described herein, wherein R⁴ is alkoxy, cyano, H or halo.

An embodiment of the present invention provides compounds according to formula I as described herein, wherein R⁵ is alkyl, halo or oxetanyl.

An embodiment of the present invention provides compounds according to formula I as described herein, wherein R⁶ is cyano or H.

An embodiment of the present invention provides compounds according to formula I as described herein, wherein R⁵ is halo and R⁶ is H.

An embodiment of the present invention provides compounds according to formula I as described herein, wherein R¹⁰ is alkyl.

An embodiment of the present invention provides compounds according to formula I as described herein, wherein when R^{7a}, R^{7b}, R^{8a} and R^{8b} are all H then X₁ is N and n is 1.

An embodiment of the present invention provides compounds according to formula I as described herein, wherein
R¹ is alkyl, cyano, cyanoalkyl, cyclopropyl, halo, haloalkoxy, haloalkyl or oxetanyl;
R² is alkoxy, H or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy, H, or halo;
R¹² is H or halo;
R¹³ is alkoxy, H, halo or haloalkoxy;
W is selected from Ring Systems A, B, C, D, or E

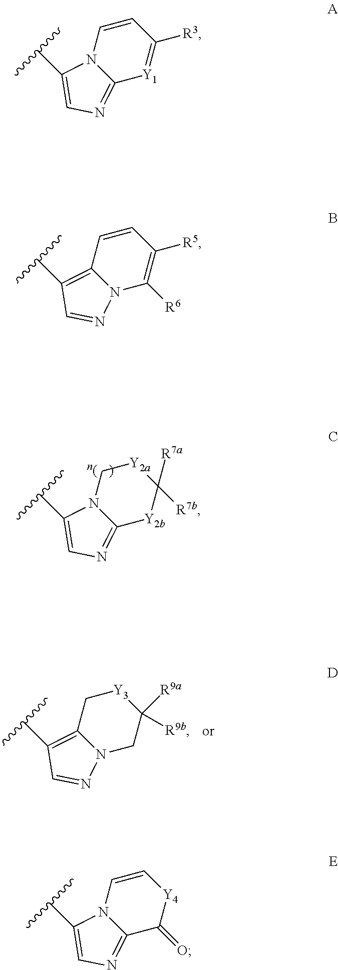

Y₁ is CR⁴ or N;
R³ is alkoxy, alkyl, cyano, cyclopropyl, H, halo, haloalkoxy, haloalkyl, hydroxyalkyl, hydroxyoxetanyl, or oxetanyl;
R⁴ alkoxy, alkyl, cyano, H, or halo;
R⁵ alkyl, halo or oxetanyl;
R⁶ cyano or H;
Y₂ₐ is CH₂ and Y_{2b} is CR^{8b} or O, wherein R^{8b} is H or alkyl;
n is 0 or 1;
R^{7a} and R^{7b} are independently selected from H, alkyl, alkoxy, or haloalkyl;
Y₃ is O or CH₂;
R^{9a} or R^{9b} are alkyl and the other is H;
Y₄ is NR¹⁰;
R¹⁰ is alkyl;
and pharmaceutically acceptable salts thereof.

An embodiment of the present invention provides compounds according to formula I as described herein, wherein
R¹ is alkyl, cyano, cyanoalkyl, cyclopropyl, halo, haloalkoxy, haloalkyl or oxetanyl;
R² is alkoxy, H or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy, H, or halo;
R¹² is H or halo;
R¹³ is alkoxy, H, halo or haloalkoxy;
W is selected from Ring Systems A, B, C, D, or E

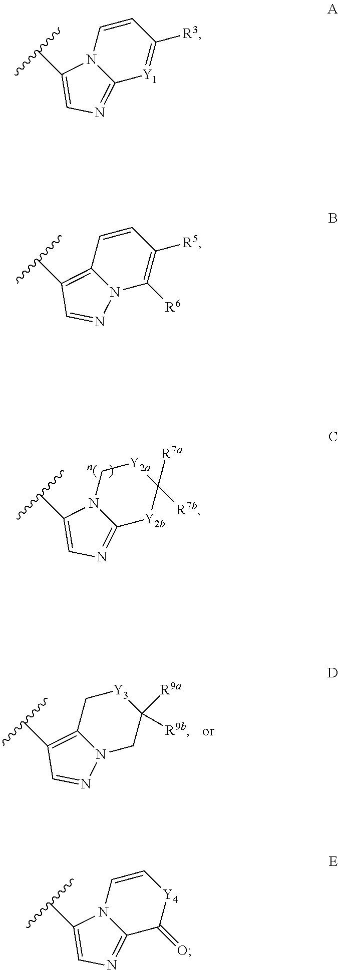

Y₁ is CR⁴ or N;
R³ is alkoxy, alkyl, cyano, cyclopropyl, H, halo, haloalkoxy, haloalkyl, hydroxyalkyl, hydroxyoxetanyl, or oxetanyl;
R⁴ alkoxy, alkyl, cyano, H, or halo;
R⁵ alkyl, halo or oxetanyl;
R⁶ cyano or H;
Y₂ₐ is CH₂ and Y_{2b} is CR^{8b} or O, wherein R^{8b} is H or alkyl;
n is 0 or 1;
R^{7a} and R^{7b} are independently selected from H, alkyl, alkoxy, or haloalkyl;
Y₃ is O or CH₂;
R^{9a} or R^{9b} are alkyl and the other is H;
Y₄ is NR¹⁰;
R¹⁰ is alkyl;
wherein when R^{7a}, R^{7b}, R^{8a} and R^{8b} are all H then X₁ is N and nis 1;
and pharmaceutically acceptable salts thereof.

An embodiment of the present invention provides compounds according to formula I as described herein, wherein
R¹ is alkyl, cyanoalkyl, cyclopropyl, halo, haloalkoxy, or haloalkyl;
R² is alkoxy or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy or H;
R¹² is H;
R¹³ is alkoxy, halo or haloalkoxy;
W is selected from Ring Systems A, B, or C

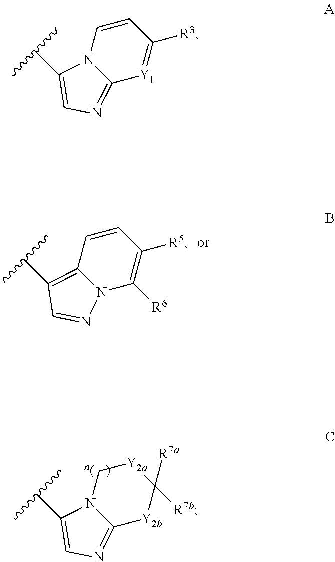

Y₁ is CR⁴ or N;
R³ is alkoxy, alkyl, cyclopropyl, halo, haloalkoxy, haloalkyl, hydroxyalkyl, or oxetanyl;
R⁴ is alkoxy, cyano, H, or halo;
R⁵ is halo;
R⁶ is H;
Y₂ₐ is CH₂ and Y_{2b} is CR^{8b} or O, wherein R^{8b} is H or alkyl;
n is 1;
R^{7a} and R^{7b} are independently selected from H, alkyl, or haloalkyl;
and pharmaceutically acceptable salts thereof.

An embodiment of the present invention provides compounds according to formula I as described herein, wherein
R¹ is alkyl, cyanoalkyl, cyclopropyl, halo, haloalkoxy, or haloalkyl;
R² is alkoxy or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³.
R¹¹ is alkoxy or H;
R¹² is H;
R¹³ is alkoxy, halo or haloalkoxy;
W is selected from Ring Systems A, B, or C

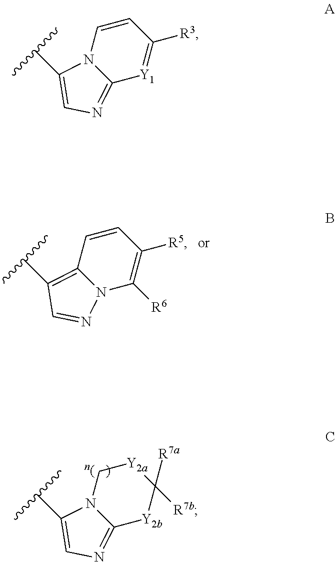

Y₁ is CR⁴ or N;
R³ is alkoxy, alkyl, cyclopropyl, halo, haloalkoxy, haloalkyl, hydroxyalkyl, or oxetanyl;
R⁴ is alkoxy, cyano, H, or halo;
R⁵ is halo;
R⁶ is H;
Y₂ₐ is CH₂ and Y_{2b} is CR^{8b} or O, wherein R^{8b} is H or alkyl;
n is 1;
R^{7a} and R^{7b} are independently selected from H, alkyl, or haloalkyl;
wherein when R^{7a}, R^{7b}, R^{8a} and R^{8b} are all H then X₁ is N and nis 1;
and pharmaceutically acceptable salts thereof.

Particular examples of compounds of formula I as described herein are selected from
7-bromo-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyridine-3-sulfonamide;
7-bromo-N-[4-(cyanomethyl)-5-fluoro-2-methoxy-phenyl]imidazo[1,2-a]pyridine-3-sulfonamide;
N-[6-(2,2-difluoroethoxy)-5-fluoro-2-methoxy-3-pyridyl]-8-methoxy-7-methyl-imidazo[1,2-a]pyridine-3-sulfonamide;
N-[6-(2,2-difluoroethoxy)-5-fluoro-2-methoxy-3-pyridyl]-7-(difluoromethoxy)imidazo[1,2-a]pyridine-3-sulfonamide;
7-chloro-N-[6-(2,2-difluoroethoxy)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-fluoro-imidazo[1,2-a]pyridine-3-sulfonamide;
7-bromo-N-[6-(difluoromethoxy)-5-fluoro-2-methoxy-3-pyridyl]imidazo[1,2-a]pyridine-3-sulfonamide;
7-chloro-N-[6-(3,3-difluoropropyl)-5-fluoro-2-methoxy-3-pyridyl]imidazo[1,2-a]pyridine-3-sulfonamide;
7-bromo-N-[5-(2,2-difluoroethyl)-3-fluoro-6-methoxy-2-pyridyl]imidazo[1,2-a]pyridine-3-sulfonamide;
7-chloro-N-[6-(2,2-difluoroethyl)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-methoxy-imidazo[1,2-a]pyridine-3-sulfonamide;
N-[6-(difluoromethoxy)-5-fluoro-2-methoxy-3-pyridyl]-7-methyl-imidazo[1,2-a]pyridine-3-sulfonamide;
N-[6-(2,2-difluoroethoxy)-5-fluoro-2-methoxy-3-pyridyl]-7-(difluoromethyl)imidazo[1,2-a]pyridine-3-sulfonamide;
N-[6-(2,2-difluoroethoxy)-5-fluoro-2-methoxy-3-pyridyl]-7-methoxy-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-ethyl-imidazo[1,2-a]pyridine-3-sulfonamide;
N-[4-(cyanomethyl)-5-fluoro-2-methoxy-phenyl]-7-cyclopropyl-imidazo[1,2-a]pyrimidine-3-sulfonamide;
N-[6-(difluoromethoxy)-5-fluoro-2-methoxy-3-pyridyl]-8-methoxy-7-methyl-imidazo[1,2-a]pyridine-3-sulfonamide;
N-[4-(cyanomethyl)-5-fluoro-2-methoxy-phenyl]-7-methyl-imidazo[1,2-a]pyridine-3-sulfonamide;
7-bromo-N-[5-(cyanomethyl)-3-fluoro-6-methoxy-2-pyridyl]imidazo[1,2-a]pyridine-3-sulfonamide;
7-chloro-N-[6-(cyanomethyl)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-5,6,7,8-tetrahydroimidazo[1,2-a]pyridine-3-sulfonamide;
7-chloro-N-[5-(2,2-difluoroethyl)-3-fluoro-6-methoxy-2-pyridyl]imidazo[1,2-a]pyridine-3-sulfonamide;
7-chloro-N-(6-cyclopropyl-5-fluoro-2-methoxy-3-pyridyl)imidazo[1,2-a]pyridine-3-sulfonamide;
7-bromo-N-[5-(difluoromethoxy)-3-fluoro-6-methoxy-2-pyridyl]imidazo[1,2-a]pyridine-3-sulfonamide;
8-cyano-N-[6-(2,2-difluoroethoxy)-5-fluoro-2-methoxy-3-pyridyl]-7-methyl-imidazo[1,2-a]pyridine-3-sulfonamide;
7-cyclopropyl-N-[6-(difluoromethoxy)-5-fluoro-2-methoxy-3-pyridyl]imidazo[1,2-a]pyrimidine-3-sulfonamide;
N-[6-(difluoromethoxy)-5-fluoro-2-methoxy-3-pyridyl]-7-methoxy-imidazo[1,2-a]pyridine-3-sulfonamide;
N-[6-(2,2-difluoroethoxy)-5-fluoro-2-methoxy-3-pyridyl]-7,8-dimethyl-5,6,7,8-tetrahydroimidazo[1,2-a]pyridine-3-sulfonamide;
N-[6-(2,2-difluoroethoxy)-5-fluoro-2-methoxy-3-pyridyl]-7-(trifluoromethyl)imidazo[1,2-a]pyridine-3-sulfonamide;
N-[4-(cyanomethyl)-2,5-difluoro-phenyl]-8-methoxy-7-methyl-imidazo[1,2-a]pyridine-3-sulfonamide;
8-cyano-7-cyclopropyl-N-[6-(2,2-difluoroethoxy)-5-fluoro-2-methoxy-3-pyridyl]imidazo[1,2-a]pyridine-3-sulfonamide;
7-bromo-N-[4-(cyanomethyl)-2,5-difluoro-phenyl]imidazo[1,2-a]pyridine-3-sulfonamide; N-[5-(cyanomethyl)-3-fluoro-6-methoxy-2-pyridyl]-7-methoxy-imidazo[1,2-a]pyridine-3-sulfonamide;
7-cyclopropyl-N-[5-(2,2-difluoroethyl)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-7-methoxy-imidazo[1,2-a]pyrimidine-3-sulfonamide;
N-[2,6-bis(difluoromethoxy)-5-fluoro-3-pyridyl]-7-chloro-imidazo[1,2-a]pyridine-3-sulfonamide;
7-chloro-N-[6-(difluoromethoxy)-5-fluoro-2-methoxy-3-pyridyl]imidazo[1,2-a]pyridine-3-sulfonamide;
7-chloro-N-[5-(difluoromethoxy)-3-fluoro-6-methoxy-2-pyridyl]imidazo[1,2-a]pyridine-3-sulfonamide;
N-[6-(2,2-difluoroethoxy)-5-fluoro-2-methoxy-3-pyridyl]-7-(trifluoromethyl)-5,6,7,8-tetrahydroimidazo[1,2-a]pyridine-3-sulfonamide;
7-chloro-N-[6-(2,2-difluoroethoxy)-5-fluoro-2-methoxy-3-pyridyl]imidazo[1,2-a]pyrimidine-3-sulfonamide;
8-cyano-N-[6-(difluoromethoxy)-5-fluoro-2-methoxy-3-pyridyl]-7-methyl-imidazo[1,2-a]pyridine-3-sulfonamide;
7-chloro-N-(5-cyclopropyl-3-fluoro-6-methoxy-2-pyridyl)imidazo[1,2-a]pyridine-3-sulfonamide;
N-[6-(2,2-difluoroethoxy)-5-fluoro-2-methoxy-3-pyridyl]-7,7-dimethyl-6,8-dihydro-5H-imidazo[1,2-a]pyridine-3-sulfonamide;
7-bromo-N-(5-bromo-4,6-dimethoxy-pyrimidin-2-yl)imidazo[1,2-a]pyridine-3-sulfonamide;
7-bromo-N-[3-fluoro-5-(2-fluoroethoxy)-6-methoxy-2-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyrimidine-3-sulfonamide;
7-cyclopropyl-N-[5-(2,2-difluoroethoxy)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-8-methyl-5,6,7,8-tetrahydroimidazo[1,2-a]pyridine-3-sulfonamide;
7-chloro-N-[5-(2,2-difluoroethoxy)-3-fluoro-6-methoxy-2-pyridyl]imidazo[1,2-a]pyridine-3-sulfonamide;
7-chloro-8-cyano-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-6,7-dihydro-5H-imidazo[2,1-b][1,3]oxazine-3-sulfonamide;
N-(4,6-dimethoxy-5-methyl-pyrimidin-2-yl)-7-methyl-imidazo[1,2-a]pyridine-3-sulfonamide;
N-[4-(cyanomethyl)-2,5-difluoro-phenyl]-7-methoxy-imidazo[1,2-a]pyridine-3-sulfonamide;
N-[2,6-bis(difluoromethoxy)-5-fluoro-3-pyridyl]-7-cyclopropyl-imidazo[1,2-a]pyrimidine-3-sulfonamide;
N-(4-bromo-2,5-difluoro-phenyl)-8-methoxy-7-methyl-imidazo[1,2-a]pyridine-3-sulfonamide;
N-[6-(2,2-difluoroethoxy)-5-fluoro-2-methoxy-3-pyridyl]-8-keto-7-methyl-imidazo[1,2-a]pyrazine-3-sulfonamide;
7-cyano-N-[6-(2,2-difluoroethoxy)-5-fluoro-2-methoxy-3-pyridyl]imidazo[1,2-a]pyridine-3-sulfonamide;
8-cyano-N-[6-(2,2-difluoroethoxy)-5-fluoro-2-methoxy-3-pyridyl]-7-methoxy-imidazo[1,2-a]pyridine-3-sulfonamide;
7-chloro-N-[4-(cyanomethyl)-2,5-difluoro-phenyl]-8-methoxy-imidazo[1,2-a]pyridine-3-sulfonamide;
N-[6-(2,2-difluoroethoxy)-5-fluoro-2-methoxy-3-pyridyl]-7-methoxy-5,6,7,8-tetrahydroimidazo[1,2-a]pyridine-3-sulfonamide;
N-[6-(2,2-difluoroethoxy)-5-fluoro-2-methoxy-3-pyridyl]-5,6,7,8-tetrahydroimidazo[1,2-a]pyridine-3-sulfonamide;
7-chloro-N-(4-(cyanomethyl)-2,5-difluorophenyl)imidazo[1,2-a]pyridine-3-sulfonamide; 8-cyano-N-[6-(2,2-difluoroethoxy)-5-fluoro-2-methoxy-3-pyridyl]-7-(difluoromethoxy)imidazo[1,2-a]pyridine-3-sulfonamide;
N-[2,6-bis(difluoromethoxy)-5-fluoro-3-pyridyl]-8-cyano-7-methyl-imidazo[1,2-a]pyridine-3-sulfonamide;
8-cyano-N-[4-(cyanomethyl)-2,5-difluoro-phenyl]-7-methyl-imidazo[1,2-a]pyridine-3-sulfonamide;
7-chloro-N-[4-(cyanomethyl)-2,5-difluoro-phenyl]-8-fluoro-imidazo[1,2-a]pyridine-3-sulfonamide;
8-cyano-N-[6-(2,2-difluoroethoxy)-5-fluoro-2-methoxy-3-pyridyl]imidazo[1,2-a]pyridine-3-sulfonamide;
7-chloro-N-[5-fluoro-2-methoxy-6-(oxetan-3-yl)-3-pyridyl]imidazo[1,2-a]pyridine-3-sulfonamide;
N-[6-(2,2-difluoroethoxy)-5-fluoro-2-methoxy-3-pyridyl]-8-methyl-imidazo[1,2-a]pyridine-3-sulfonamide;
N-[6-(2,2-difluoroethoxy)-5-fluoro-2-methoxy-3-pyridyl]-6-methyl-6,7-dihydro-5H-pyrrol [1,2-a]imidazole-3-sulfonamide;
N-[4-(cyanomethyl)-2,5-difluoro-phenyl]-7-methoxy-imidazo[1,2-a]pyrimidine-3-sulfonamide;
7-chloro-N-[5-(2-fluoroethoxy)-4-methoxy-pyrimidin-2-yl]imidazo[1,2-a]pyridine-3-sulfonamide;
N-[6-(2,2-difluoroethoxy)-5-fluoro-2-methoxy-3-pyridyl]-7-methyl-6,7-dihydro-5H-pyrrol [1,2-a]imidazole-3-sulfonamide;
N-[4-(cyanomethyl)-2,5-difluoro-phenyl]-7-methyl-imidazo[1,2-a]pyrimidine-3-sulfonamide;
6-bromo-N-[6-(cyanomethyl)-5-fluoro-2-methoxy-3-pyridyl]pyrazolo[1,5-a]pyridine-3-sulfonamide;
6-bromo-N-[6-(2,2-difluoroethoxy)-5-fluoro-2-methoxy-3-pyridyl]pyrazolo[1,5-a]pyridine-3-sulfonamide;
6-bromo-N-[5-(cyanomethyl)-3-fluoro-6-methoxy-2-pyridyl]pyrazolo[1,5-a]pyridine-3-sulfonamide;
6-bromo-N-[3-fluoro-5-(2-fluoroethoxy)-6-methoxy-2-pyridyl]pyrazolo[1,5-a]pyridine-3-sulfonamide;
6-bromo-N-[5-(2,2-difluoroethoxy)-3-fluoro-6-methoxy-2-pyridyl]pyrazolo[1,5-a]pyridine-3-sulfonamide;
6-bromo-N-(6-cyclopropyl-5-fluoro-2-methoxy-3-pyridyl)pyrazolo[1,5-a]pyridine-3-sulfonamide;
N-[6-(2,2-difluoroethoxy)-5-fluoro-2-methoxy-3-pyridyl]-6-methyl-4,5,6,7-tetrahydropyrazolo[1,5-a]pyridine-3-sulfonamide;
7-cyano-N-[6-(2,2-difluoroethoxy)-5-fluoro-2-methoxy-3-pyridyl]-6-methyl-pyrazolo[1,5-a]pyridine-3-sulfonamide;
6-bromo-N-[4-(difluoromethoxy)-2,5-difluoro-phenyl]pyrazolo[1,5-a]pyridine-3-sulfonamide;
6-bromo-N-(4-bromo-2,5-difluoro-phenyl)pyrazolo[1,5-a]pyridine-3-sulfonamide;
6-bromo-N-[5-fluoro-2-methoxy-6-(oxetan-3-yl)-3-pyridyl]pyrazolo[1,5-a]pyridine-3-sulfonamide;
N-[6-(2,2-difluoroethoxy)-5-fluoro-2-methoxy-3-pyridyl]-6-methyl-6,7-dihydro-4H-pyrazolo[5,1-c][1,4]oxazine-3-sulfonamide;
N-[4-(cyanomethyl)-5-fluoro-2-methoxy-phenyl]-7-cyclopropyl-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyridine-3-sulfonamide;
7-cyclopropyl-N-[6-(difluoromethoxy)-5-fluoro-2-methoxy-3-pyridyl]imidazo[1,2-a]pyridine-3-sulfonamide;
N-[5-(cyanomethyl)-3-fluoro-6-methoxy-2-pyridyl]-7-cyclopropyl-imidazo[1,2-a]pyridine-3-sulfonamide;
7-cyclopropyl-N-[5-(difluoromethoxy)-3-fluoro-6-methoxy-2-pyridyl]imidazo[1,2-a]pyrimidine-3-sulfonamide;
N-(4-cyano-2,5-difluoro-phenyl)-8-methoxy-7-methyl-imidazo[1,2-a]pyridine-3-sulfonamide;
6-bromo-N-[5-(difluoromethoxy)-3-fluoro-6-methoxy-2-pyridyl]pyrazolo[1,5-a]pyridine-3-sulfonamide;
N-[6-(2,2-difluoroethoxy)-5-fluoro-2-methoxy-3-pyridyl]-7-(oxetan-3-yl)imidazo[1,2-a]pyridine-3-sulfonamide;
N-[4-(cyanomethyl)-5-fluoro-2-methoxy-phenyl]-7-(oxetan-3-yl)imidazo[1,2-a]pyridine-3-sulfonamide;
N-[5-(2,2-difluoroethyl)-3-fluoro-6-methoxy-2-pyridyl]-7-(oxetan-3-yl)imidazo[1,2-a]pyridine-3-sulfonamide;
N-[6-(2,2-difluoroethoxy)-5-fluoro-2-methoxy-3-pyridyl]-7-(oxetan-3-yl)imidazo[1,2-a]pyrimidine-3-sulfonamide;
N-[6-(2,2-difluoroethoxy)-5-fluoro-2-methoxy-3-pyridyl]-6-(oxetan-3-yl)pyrazolo[1,5-a]pyridine-3-sulfonamide;
7-bromo-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-(1-hydroxy-1-methyl-ethyl)imidazo[1,2-a]pyridine-3-sulfonamide;
N-[6-(2,2-difluoroethoxy)-5-fluoro-2-methoxy-3-pyridyl]-7-(3-hydroxyoxetan-3-yl)imidazo[1,2-a]pyridine-3-sulfonamide;
and pharmaceutically acceptable salts thereof.

Preferred examples of compounds of formula I as described herein are selected from
7-bromo-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyridine-3-sulfonamide;
7-bromo-N-[4-(cyanomethyl)-5-fluoro-2-methoxy-phenyl]imidazo[1,2-a]pyridine-3-sulfonamide;
N-[6-(2,2-difluoroethoxy)-5-fluoro-2-methoxy-3-pyridyl]-8-methoxy-7-methyl-imidazo[1,2-a]pyridine-3-sulfonamide;
N-[6-(2,2-difluoroethoxy)-5-fluoro-2-methoxy-3-pyridyl]-7-(difluoromethoxy)imidazo[1,2-a]pyridine-3-sulfonamide;
7-chloro-N-[6-(2,2-difluoroethoxy)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-fluoro-imidazo[1,2-a]pyridine-3-sulfonamide;
7-bromo-N-[6-(difluoromethoxy)-5-fluoro-2-methoxy-3-pyridyl]imidazo[1,2-a]pyridine-3-sulfonamide;
7-chloro-N-[6-(3,3-difluoropropyl)-5-fluoro-2-methoxy-3-pyridyl]imidazo[1,2-a]pyridine-3-sulfonamide;
7-bromo-N-[5-(2,2-difluoroethyl)-3-fluoro-6-methoxy-2-pyridyl]imidazo[1,2-a]pyridine-3-sulfonamide;
7-chloro-N-[6-(2,2-difluoroethyl)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-methoxy-imidazo[1,2-a]pyridine-3-sulfonamide;
N-[6-(difluoromethoxy)-5-fluoro-2-methoxy-3-pyridyl]-7-methyl-imidazo[1,2-a]pyridine-3-sulfonamide;
N-[6-(2,2-difluoroethoxy)-5-fluoro-2-methoxy-3-pyridyl]-7-(difluoromethyl)imidazo[1,2-a]pyridine-3-sulfonamide;
N-[6-(2,2-difluoroethoxy)-5-fluoro-2-methoxy-3-pyridyl]-7-methoxy-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-ethyl-imidazo[1,2-a]pyridine-3-sulfonamide;
N-[4-(cyanomethyl)-5-fluoro-2-methoxy-phenyl]-7-cyclopropyl-imidazo[1,2-a]pyrimidine-3-sulfonamide;
N-[6-(difluoromethoxy)-5-fluoro-2-methoxy-3-pyridyl]-8-methoxy-7-methyl-imidazo[1,2-a]pyridine-3-sulfonamide;
N-[4-(cyanomethyl)-5-fluoro-2-methoxy-phenyl]-7-methyl-imidazo[1,2-a]pyridine-3-sulfonamide;
7-bromo-N-[5-(cyanomethyl)-3-fluoro-6-methoxy-2-pyridyl]imidazo[1,2-a]pyridine-3-sulfonamide;
7-chloro-N-[6-(cyanomethyl)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-5,6,7,8-tetrahydroimidazo[1,2-a]pyridine-3-sulfonamide;
7-chloro-N-[5-(2,2-difluoroethyl)-3-fluoro-6-methoxy-2-pyridyl]imidazo[1,2-a]pyridine-3-sulfonamide;
7-chloro-N-(6-cyclopropyl-5-fluoro-2-methoxy-3-pyridyl)imidazo[1,2-a]pyridine-3-sulfonamide;
7-bromo-N-[5-(difluoromethoxy)-3-fluoro-6-methoxy-2-pyridyl]imidazo[1,2-a]pyridine-3-sulfonamide;
8-cyano-N-[6-(2,2-difluoroethoxy)-5-fluoro-2-methoxy-3-pyridyl]-7-methyl-imidazo[1,2-a]pyridine-3-sulfonamide;
7-cyclopropyl-N-[6-(difluoromethoxy)-5-fluoro-2-methoxy-3-pyridyl]imidazo[1,2-a]pyrimidine-3-sulfonamide;
N-[6-(difluoromethoxy)-5-fluoro-2-methoxy-3-pyridyl]-7-methoxy-imidazo[1,2-a]pyridine-3-sulfonamide;
N-[6-(2,2-difluoroethoxy)-5-fluoro-2-methoxy-3-pyridyl]-7,8-dimethyl-5,6,7,8-tetrahydroimidazo[1,2-a]pyridine-3-sulfonamide;
N-[6-(2,2-difluoroethoxy)-5-fluoro-2-methoxy-3-pyridyl]-7-(trifluoromethyl)imidazo[1,2-a]pyridine-3-sulfonamide;
N-[4-(cyanomethyl)-2,5-difluoro-phenyl]-8-methoxy-7-methyl-imidazo[1,2-a]pyridine-3-sulfonamide;
8-cyano-7-cyclopropyl-N-[6-(2,2-difluoroethoxy)-5-fluoro-2-methoxy-3-pyridyl]imidazo[1,2-a]pyridine-3-sulfonamide;
7-bromo-N-[4-(cyanomethyl)-2,5-difluoro-phenyl]imidazo[1,2-a]pyridine-3-sulfonamide;
N-[5-(cyanomethyl)-3-fluoro-6-methoxy-2-pyridyl]-7-methoxy-imidazo[1,2-a]pyridine-3-sulfonamide;
7-cyclopropyl-N-[5-(2,2-difluoroethyl)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-7-methoxy-imidazo[1,2-a]pyrimidine-3-sulfonamide;
N-[2,6-bis(difluoromethoxy)-5-fluoro-3-pyridyl]-7-chloro-imidazo[1,2-a]pyridine-3-sulfonamide;
7-chloro-N-[6-(difluoromethoxy)-5-fluoro-2-methoxy-3-pyridyl]imidazo[1,2-a]pyridine-3-sulfonamide;
7-chloro-N-[5-(difluoromethoxy)-3-fluoro-6-methoxy-2-pyridyl]imidazo[1,2-a]pyridine-3-sulfonamide;
N-[6-(2,2-difluoroethoxy)-5-fluoro-2-methoxy-3-pyridyl]-7-(trifluoromethyl)-5,6,7,8-tetrahydroimidazo[1,2-a]pyridine-3-sulfonamide;
7-chloro-N-[6-(2,2-difluoroethoxy)-5-fluoro-2-methoxy-3-pyridyl]imidazo[1,2-a]pyrimidine-3-sulfonamide;
8-cyano-N-[6-(difluoromethoxy)-5-fluoro-2-methoxy-3-pyridyl]-7-methyl-imidazo[1,2-a]pyridine-3-sulfonamide;
7-chloro-N-(5-cyclopropyl-3-fluoro-6-methoxy-2-pyridyl)imidazo[1,2-a]pyridine-3-sulfonamide;
N-[6-(2,2-difluoroethoxy)-5-fluoro-2-methoxy-3-pyridyl]-7,7-dimethyl-6,8-dihydro-5H-imidazo[1,2-a]pyridine-3-sulfonamide;
7-bromo-N-(5-bromo-4,6-dimethoxy-pyrimidin-2-yl)imidazo[1,2-a]pyridine-3-sulfonamide;
7-bromo-N-[3-fluoro-5-(2-fluoroethoxy)-6-methoxy-2-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyrimidine-3-sulfonamide;
7-cyclopropyl-N-[5-(2,2-difluoroethoxy)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-8-methyl-5,6,7,8-tetrahydroimidazo[1,2-a]pyridine-3-sulfonamide;
7-chloro-N-[5-(2,2-difluoroethoxy)-3-fluoro-6-methoxy-2-pyridyl]imidazo[1,2-a]pyridine-3-sulfonamide;
7-chloro-8-cyano-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-6,7-dihydro-5H-imidazo[2,1-b][1,3]oxazine-3-sulfonamide;
N-(4,6-dimethoxy-5-methyl-pyrimidin-2-yl)-7-methyl-imidazo[1,2-a]pyridine-3-sulfonamide;
6-bromo-N-(6-cyclopropyl-5-fluoro-2-methoxy-3-pyridyl)pyrazolo[1,5-a]pyridine-3-sulfonamide;
N-[4-(cyanomethyl)-5-fluoro-2-methoxy-phenyl]-7-cyclopropyl-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyridine-3-sulfonamide;
7-cyclopropyl-N-[6-(difluoromethoxy)-5-fluoro-2-methoxy-3-pyridyl]imidazo[1,2-a]pyridine-3-sulfonamide;
N-[5-(cyanomethyl)-3-fluoro-6-methoxy-2-pyridyl]-7-cyclopropyl-imidazo[1,2-a]pyridine-3-sulfonamide;
7-cyclopropyl-N-[5-(difluoromethoxy)-3-fluoro-6-methoxy-2-pyridyl]imidazo[1,2-a]pyrimidine-3-sulfonamide;
6-bromo-N-[5-(difluoromethoxy)-3-fluoro-6-methoxy-2-pyridyl]pyrazolo[1,5-a]pyridine-3-sulfonamide;
N-[6-(2,2-difluoroethoxy)-5-fluoro-2-methoxy-3-pyridyl]-7-(oxetan-3-yl)imidazo[1,2-a]pyridine-3-sulfonamide;
7-bromo-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-(1-hydroxy-1-methyl-ethyl)imidazo[1,2-a]pyridine-3-sulfonamide;
and pharmaceutical salts thereof.

Processes for the manufacture of compounds of formula I as described herein are an object of the invention.

The present compounds of formula I and their pharmaceutically acceptable salts can be prepared by methods known in the art, for example, by processes described below, which process comprises reacting a compound of formula V with a compound of formula VI in the presence of a base to provide a compound of formula I,

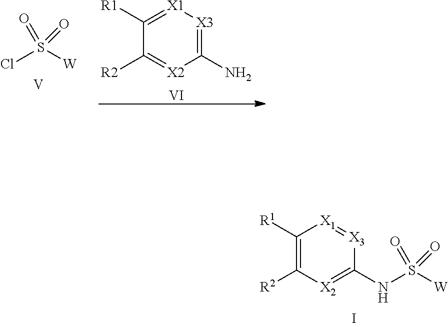

wherein R¹, R², X₁, X₂, X₃ and W are as described above.

### General Synthetic Schemes

The compounds of formula I may be prepared in accordance with the process variant described above and with the following Scheme 1. The starting materials are commercially available or may be prepared in accordance with known methods.

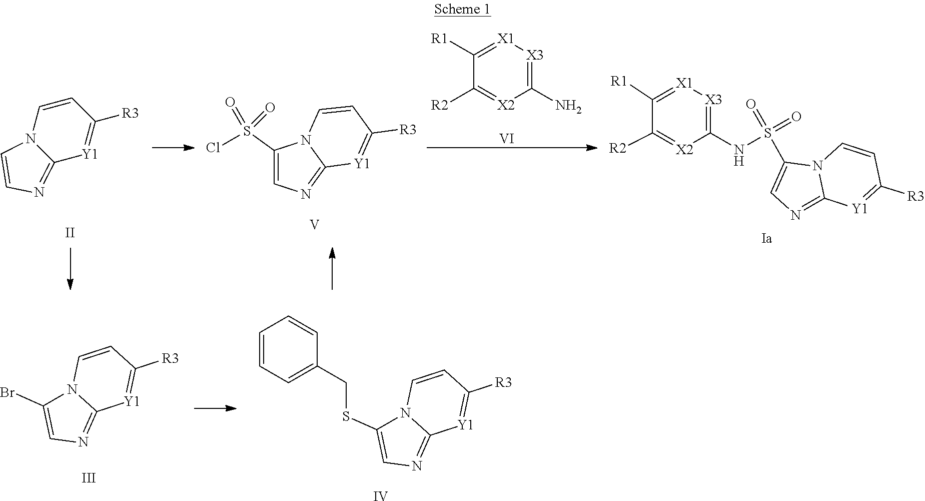

Compounds of general formula Ia can be prepared by reacting sulfonylchloride V with 2-amino-pyrimidine or 2-amino-pyridine or 3-amino-pyridine or aniline VI in the presence of a base (e.g. pyridine). Sulfonylchloride V can be prepared from intermediate II in the presence of chlorosulfonylating agent (e.g. chlorosulfonic acid or trimethylsilyl chlorosulfonate). Alternatively, sulfonylchloride V can be prepared from intermediate IV in presence of chlorinating agent like NCS in aqueous solution. Intermediate IV can be prepared from intermediate III using Buchwald-Hartwig type cross coupling using palladium catalyst system such as Pd(OAc)₂ or Pd₂(dba)₃/xantphos or XPhos and an excess of a base such as DIPEA or Cs₂CO₃ at elevated temperatures in solvents such as dioxane or toluene. Intermediate III can be obtained from intermediate II in presence of brominating agent like NBS in solvents such as MeCN, EtOAc or DMF.

The compounds of formula VII may be prepared in accordance with the process variant described above and with the following Scheme 2. The starting materials are commercially available or may be prepared in accordance with known methods.

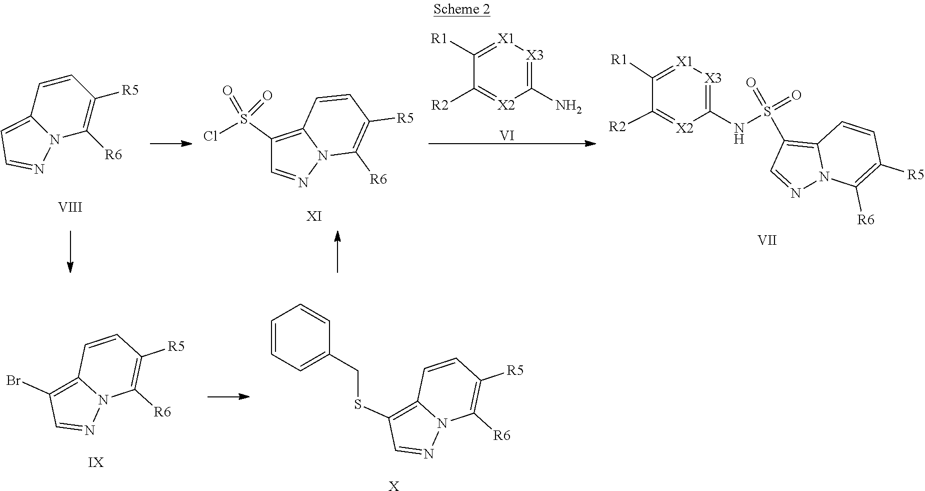

Compounds of general formula VII can be prepared by reacting sulfonylchloride XI with 2-amino-pyrimidine or 2-amino-pyridine or 3-amino-pyridine or aniline VI in the presence of a base (e.g. pyridine). Sulfonylchloride XI can be prepared from intermediate VIII in the presence of chlorosulfonylating agent (e.g. chlorosulfonic acid or trimethylsilyl chlorosulfonate). Alternatively, sulfonylchloride XI can be prepared from intermediate X in presence of chlorinating agent like NCS in aqueous solution. Intermediate X can be prepared from intermediate IX using Buchwald-Hartwig type cross coupling using palladium catalyst system such as Pd(OAc)₂ or Pd₂(dba)₃/xantphos or XPhos and an excess of a base such as DIPEA or Cs₂CO₃ at elevated temperatures in solvents such as dioxane or toluene. Intermediate IX can be obtained from intermediate VIII in presence of brominating agent like NBS in solvents such as MeCN, EtOAc or DMF.

The compounds of formula XII may be prepared in accordance with the process variant described above and with the following Scheme 3. The starting materials are commercially available or may be prepared in accordance with known methods.

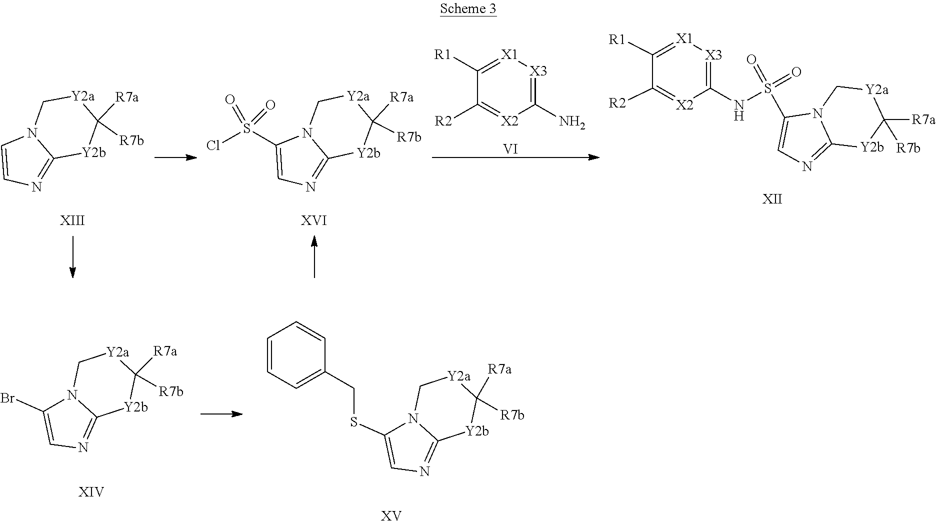

Compounds of general formula XII can be prepared by reacting sulfonylchloride XVI with 2-amino-pyrimidine or 2-amino-pyridine or 3-amino-pyridine or aniline VI in the presence of a base (e.g. pyridine). Sulfonylchloride XVI can be prepared from intermediate XIII in the presence of chlorosulfonylating agent (e.g. chlorosulfonic acid or trimethylsilyl chlorosulfonate). Alternatively, sulfonylchloride XVI can be prepared from intermediate XV in presence of chlorinating agent like NCS in aqueous solution. Intermediate XV can be prepared from intermediate XIV using Buchwald-Hartwig type cross coupling using palladium catalyst system such as Pd(OAc)₂ or Pd₂(dba)₃/xantphos or xphos and an excess of a base such as DIPEA or Cs₂CO₃ at elevated temperatures in solvents such as dioxane or toluene. Intermediate XIV can be obtained from intermediate XIII in presence of brominating agent like NBS in solvents such as MeCN, EtOAc or DMF.

The compounds of formula XVII may be prepared in accordance with the process variant described above and with the following Scheme 4. The starting materials are commercially available or may be prepared in accordance with known methods.

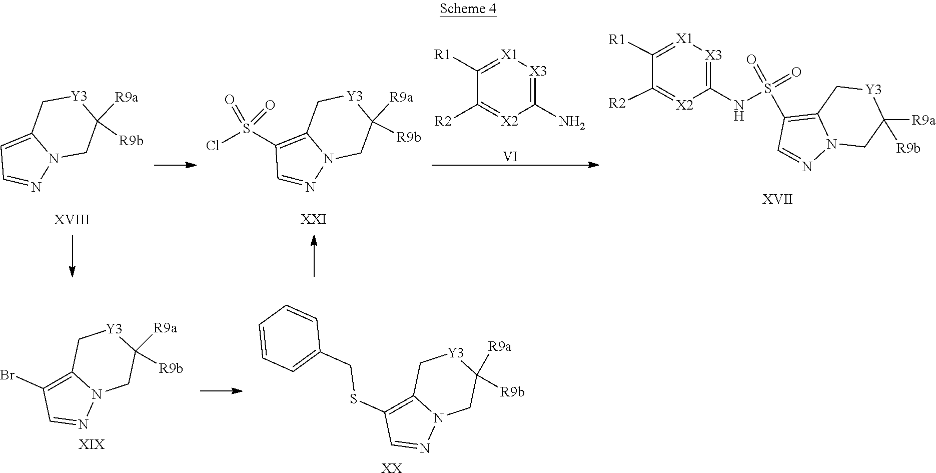

Compounds of general formula XVII can be prepared by reacting sulfonylchloride XXI with 2-amino-pyrimidine or 2-amino-pyridine or 3-amino-pyridine or aniline VI in the presence of a base (e.g. pyridine). Sulfonylchloride XXI can be prepared from intermediate XVIII in the presence of chlorosulfonylating agent (e.g. chlorosulfonic acid or trimethylsilyl chlorosulfonate). Alternatively, sulfonylchloride XXI can be prepared from intermediate XX in presence of chlorinating agent like NCS in aqueous solution. Intermediate XX can be prepared from intermediate XIX using Buchwald-Hartwig type cross coupling using palladium catalyst system such as Pd(OAc)₂ or Pd₂(dba)₃/xantphos or xphos and an excess of a base such as DIPEA or Cs₂CO₃ at elevated temperatures in solvents such as dioxane or toluene. Intermediate XIX can be obtained from intermediate XVIII in presence of brominating agent like NBS in solvents such as MeCN, EtOAc or DMF.

The compounds of formula XXII may be prepared in accordance with the process variant described above and with the following Scheme 5. The starting materials are commercially available or may be prepared in accordance with known methods.

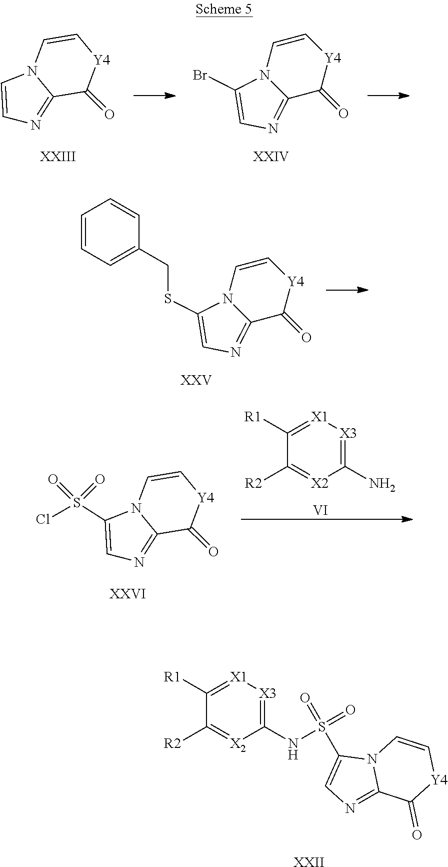

Compounds of general formula XXII can be prepared by reacting sulfonylchloride XXVI with 2-amino-pyrimidine or 2-amino-pyridine or 3-amino-pyridine or aniline VI in the presence of a base (e.g. pyridine). Sulfonylchloride XXVI can be prepared from intermediate XXV in presence of chlorinating agent like NCS in aqueous solution. Intermediate XXV can be prepared from intermediate XXIV using Buchwald-Hartwig type cross coupling using palladium catalyst system such as Pd(OAc)₂ or Pd₂(dba)₃/xantphos or xphos and an excess of a base such as DIPEA or Cs₂CO₃ at elevated temperatures in solvents such as dioxane or toluene. Intermediate XXIV can be obtained from intermediate XXIII in presence of brominating agent like NBS in solvents such as MeCN, EtOAc or DMF.

2-Amino-pyrimidines or 2-amino-pyridines or 3-amino-pyridines or anilines VI are either commercially available or may be prepared in accordance to literature or to procedures described in this patent. The starting materials are commercially available or may be prepared in accordance with known methods.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

The invention also relates in particular to:
A compound of formula I for use as therapeutically active substance;
A compound of formula I for use in the treatment of a disease modulated by GPR17;
Likewise an object of the present invention is a pharmaceutical composition comprising a compound according to formula I as described herein and a therapeutically inert carrier.

The use of a compound of formula I for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the treatment or prophylaxis of multiple sclerosis, Alzheimer's disease, Parkinson's disease, or Huntington's disease.

A particular embodiment of the invention is the use of a compound of formula I for the treatment or prophylaxis of multiple sclerosis.

The use of a compound of formula I for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the preparation of a medicament for the treatment or prophylaxis of multiple sclerosis, Alzheimer's disease, Parkinson's disease, or Huntington's disease.

A particular embodiment of the invention is the use of a compound of formula I for the preparation of a medicament for the treatment or prophylaxis of multiple sclerosis.

A compound according to formula I for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is a compound of formula I for use in the treatment or prophylaxis of multiple sclerosis, Alzheimer's disease, Parkinson's disease, or Huntington's disease.

A particular embodiment of the invention is a compound according to formula I for use in the treatment or prophylaxis of multiple sclerosis.

A method for the treatment or propylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity, which method comprises administering an effective amount of a compound of formula I to a patient in need thereof.

An embodiment of the present invention is a method for the treatment or prophylaxis of multiple sclerosis, Alzheimer's disease, Parkinson's disease, or Huntington's disease, which method comprises administering an effective amount of a compound of formula I to a patient in need thereof.

A particular embodiment of the invention is a method for the treatment or prophylaxis of multiple sclerosis, which method comprises administering an effective amount of a compound of formula I to a patient in need thereof.

Also an embodiment of the present invention provides compounds of formula I as described herein, when manufactured according to any one of the described processes.

### Assay Procedures

### GPR17 CAMP Assay Protocol:

CHO-K1 cells stably expressing vector containing untagged human GPR17 short isoform (Roche) were cultured at 37° C./5% CO₂ in DMEM (Dulbecco's Modified Eagle Medium): F-12 (1:1) supplemented with 10% foetal bovine serum and 400 μg/ml Geneticin.

Changes in intracellular cyclic adenosine monophosphate (cAMP) levels were quantified using the Nano-TRF Detection Assay kit (Roche Diagnostics, Cat. No. 05214386001). This assay allows for direct cAMP quantification in a homogeneous solution. CAMP is detected based on time-resolved fluorescence energy transfer (TR-FRET) and competitive binding of ruthenylated cAMP and endogenous cAMP to an anti-cAMP monoclonal antibody labeled with AlexaFluor-700. The Ruthenium complex serves as the FRET donor and transfers energy to AlexaFluor-700. The FRET signal is inversely proportional to the CAMP concentration.

CHO-GPR17S cells were detached with Accutase and resuspended in assay buffer consisting of Hank's Balanced Salt Solution (HBSS), 10 mM HEPES (4-(2-hydroxyethyl) piperazine-1-ethanesulfonic acid solution) and 0.1% bovine serum albumin (pH 7.4). The cells were seeded in black 384-well plates (Corning) at a density of 10′000 cells/20 μl assay buffer until the addition of compounds.

Test antagonist compounds were serially diluted in dimethyl sulfoxide (DMSO) and spotted in 384-well plates. The compounds were then diluted in HBSS buffer supplemented with an EC80 concentration of MDL29,951 (3-(2-Carboxy-4,6-dichloroindol-3-yl) propionic acid) (GPR17 agonist) plus 3-Isobutyl-1-methylxanthine (IBMX) (0.5 mM final concentration) and added to the cells at room temperature. Forskolin (15 μM final concentration) was added 5 minutes after the test compounds and the cells were incubated at room temperature for 30 minutes. The assay was stopped by adding cAMP detection mix (containing detergents for cell lysis) for 90 minutes at room temperature.

Cellular cAMP was measured using a Paradigm reader (Molecular Devices). The raw data was used to calculate the FRET signal based on the assay's P-factor as per cAMP kit instructions. The data was normalized to the maximal activity of a reference antagonist and dose response curves were fitted to the percent activity of the test compounds using a sigmoidal dose response model (Genedata Screener).

Results in the hGPR17 cAMP assay are provided for compounds of formula I in Table 1

TABLE 1

| | hGPR17 cAMP |
|---|---|
| Example Number | IC50 [μM] |
| 1 | 0.005 |
| 2 | 0.007 |
| 3 | 0.007 |
| 4 | 0.008 |
| 5 | 0.008 |
| 6 | 0.009 |
| 7 | 0.009 |
| 8 | 0.009 |
| 9 | 0.011 |
| 10 | 0.011 |
| 11 | 0.014 |
| 12 | 0.016 |
| 13 | 0.018 |
| 14 | 0.018 |
| 15 | 0.018 |
| 16 | 0.018 |
| 17 | 0.018 |
| 18 | 0.019 |
| 19 | 0.019 |
| 20 | 0.021 |
| 21 | 0.021 |
| 22 | 0.023 |
| 23 | 0.023 |
| 24 | 0.023 |
| 25 | 0.023 |
| 26 | 0.030 |
| 27 | 0.031 |
| 28 | 0.031 |
| 29 | 0.033 |
| 30 | 0.035 |
| 31 | 0.038 |
| 32 | 0.040 |
| 33 | 0.042 |
| 34 | 0.045 |
| 35 | 0.046 |
| 36 | 0.052 |
| 37 | 0.056 |
| 38 | 0.057 |
| 39 | 0.058 |
| 40 | 0.064 |
| 41 | 0.066 |
| 42 | 0.072 |
| 43 | 0.075 |
| 44 | 0.076 |
| 45 | 0.076 |
| 46 | 0.082 |
| 47 | 0.083 |
| 48 | 0.085 |
| 49 | 0.089 |
| 50 | 0.098 |
| 51 | 0.099 |
| 52 | 0.099 |
| 53 | 0.101 |
| 54 | 0.112 |
| 55 | 0.116 |
| 56 | 0.132 |
| 57 | 0.137 |
| 58 | 0.174 |
| 59 | 0.176 |
| 60 | 0.179 |
| 61 | 0.181 |
| 62 | 0.217 |
| 63 | 0.217 |
| 64 | 0.251 |
| 65 | 0.270 |
| 66 | 0.289 |
| 67 | 0.301 |
| 68 | 0.329 |
| 69 | 0.393 |
| 70 | 0.407 |
| 71 | 0.413 |
| 72 | 0.449 |
| 73 | 0.876 |
| 74 | 0.897 |
| 75 | 0.974 |
| 76 | 1.028 |
| 77 | 0.006 |
| 78 | 0.010 |
| 79 | 0.020 |
| 80 | 0.026 |
| 81 | 0.049 |
| 82 | 0.100 |
| 83 | 0.100 |
| 84 | 0.130 |
| 85 | 0.251 |
| 86 | 0.320 |
| 87 | 0.367 |
| 88 | 0.692 |
| 89 | 0.139 |
| 90 | 0.115 |
| 91 | 0.211 |
| 92 | 0.115 |
| 93 | 0.004 |
| 94 | 0.009 |
| 95 | 0.013 |
| 96 | 0.038 |
| 97 | 0.023 |
| 98 | 0.819 |
| 99 | 0.008 |
| 100 | 0.037 |
| 101 | 0.179 |
| 102 | 0.201 |
| 103 | 0.492 |
| 104 | 0.306 |
| 105 | 0.027 |
| 106 | 0.038 |
| 107 | 0.101 |
| 108 | 0.016 |
| 109 | 0.035 |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen or argon atmosphere if not specified otherwise.

### Intermediates A

The intermediates are either commercially available or have been prepared following procedures described in the indicated patent applications or novel and corresponding experimental procedures are described in the end of the table:

| | | | Patent |
|---|---|---|---|
| Intermediate | | | application |
| number | Structure | Name | number or CAS |
| A1 | 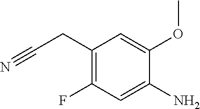 | 2-(4-amino-2-fluoro-5- methoxy-phenyl)acetonitrile | |
| A2 | 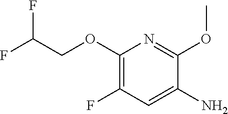 | 6-(2,2-difluoroethoxy)-5- fluoro-2-methoxy-pyridin-3- amine | WO2019243303 |
| A3 | 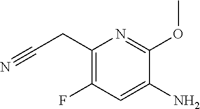 | 2-(5-amino-3-fluoro-6- methoxy-2- pyridyl)acetonitrile | |
| A4 | 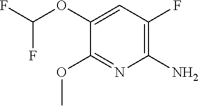 | 5-(difluoromethoxy)-3-fluoro- 6-methoxy-pyridin-2-amine | |
| A5 | 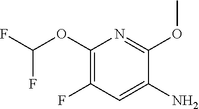 | 6-(difluoromethoxy)-5-fluoro- 2-methoxy-pyridin-3-amine | WO2019243303 |
| A6 | 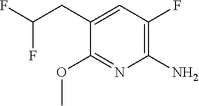 | 5-(2,2-difluoroethyl)-3- fluoro-6-methoxy-pyridin-2- amine | |
| A7 | 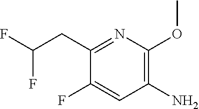 | 6-(2,2-difluoroethyl)-5- fluoro-2-methoxy-pyridin-3- amine | |
| A8 | 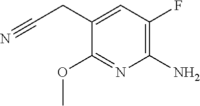 | 2-(6-amino-5-fluoro-2- methoxy-3- pyridyl)acetonitrile | WO2018122232 |
| A9 | 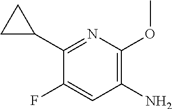 | 6-cyclopropyl-5-fluoro-2- methoxy-pyridin-3-amine | WO2019243303 |
| A10 | 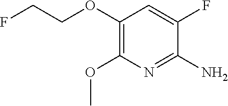 | 3-fluoro-5-(2-fluoroethoxy)- 6-methoxy-pyridin-2-amine | |
| A11 | 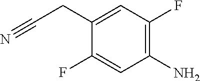 [SMILES: N#CCc1cc(F)c(N)cc1F] | 2-(4-amino-2,5-difluoro- phenyl)acetonitrile | WO2018122232 |
| A12 | 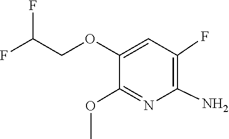 | 5-(2,2-difluoroethoxy)-3- fluoro-6-methoxy-pyridin-2- amine | WO2019243398 |
| A13 | 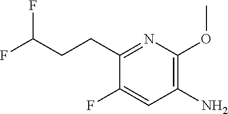 | 6-(3,3-difluoropropyl)-5- fluoro-2-methoxy-pyridin-3- amine | |
| A14 | 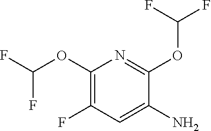 [SMILES: Nc1cc(F)c(OC(F)F)nc1OC(F)F] | 2,6-bis(difluoromethoxy)-5- fluoro-pyridin-3-amine | |
| A15 | 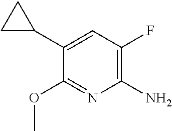 | 5-cyclopropyl-3-fluoro-6- methoxy-pyridin-2-amine | |
| A16 | 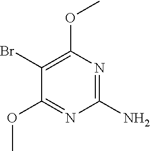 | 5-bromo-4,6-dimethoxy- pyrimidin-2-amine | |
| A17 | 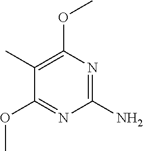 | 4,6-dimethoxy-5-methyl- pyrimidin-2-amine | |
| A18 | 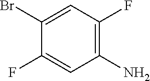 | 4-bromo-2,5-difluoro-aniline | 112279-60-4 |
| A19 | 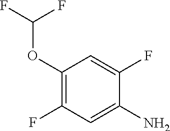 [SMILES: Nc1cc(F)c(OC(F)F)cc1F] | 4-(difluoromethoxy)-2,5- difluoro-aniline | WO2018122232 |
| A20 | 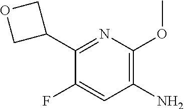 | 5-fluoro-2-methoxy-6- (oxetan-3-yl)pyridin-3-amine | |
| A21 | 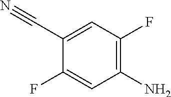 [SMILES: N#Cc1cc(F)c(N)cc1F] | 4-amino-2,5-difluoro- benzonitrile | 112279-61-5 |
| A22 | 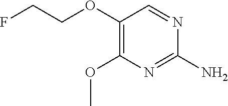 | 5-(2-fluoroethoxy)-4- methoxy-pyrimidin-2-amine | |
| A23 | 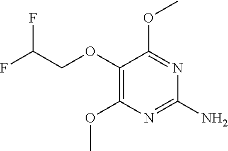 [SMILES: COc1nc(N)nc(OC)c1OCC(F)F] | 5-(2,2-difluoroethoxy)-4,6- dimethoxy-pyrimidin-2- amine | |
| A24 | 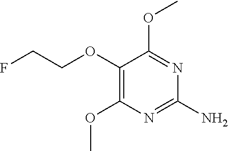 | 5-(2-fluoroethoxy)-4,6- dimethoxy-pyrimidin-2- amine | |

### Intermediate A1:2-(4-amino-2-fluoro-5-methoxy-phenyl acetonitrile

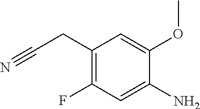

To a stirred solution of (4-bromo-5-fluoro-2-methoxy-phenyl)amine (2 g, 9.09 mmol, CAS: 330794-03-1), 4-(4,4,5,5-tetramethyl-1,3,2-dioxaborolan-2-yl) isoxazole (2.13 g, 10.91 mmol) and 1 M aqueous potassium fluoride (27.27 mL, 27.27 mmol) in N-methyl-2-pyrrolidinone under argon was added 1,1′-bis(diphenylphosphino) ferrocene-palladium (II) dichloride dichloromethane complex (751.43 mg, 0.909 mmol). The reaction mixture was stirred at 90° C. for 4 h before another equivalent of 4-(4,4,5,5-tetramethyl-1,3,2-dioxaborolan-2-yl) isoxazole (1.77 g, 9.09 mmol) was added. After additional 5 h at 90° C. 1 M aqueous potassium fluoride (9.09 mL, 9.09 mmol) was added and the reaction mixture was allowed to stir over night at 90° C. The reaction mixture was cooled to room temperature, diluted with water and extracted with ethyl acetate. The organic layers were combined, dried over anhydrous sodium sulfate, filtered and concentrated in vacuo. The crude material was purified by flash chromatography over silica gel using a gradient ethyl acetate/heptane 0-50% to provide the title compound as a yellow oil (536 mg, 32%). MS (ESI): m/z=181.1 [M+H]⁺

### Intermediate A3:2-(5-amino-3-fluoro-6-methoxy-2-pyridyl)acetonitrile

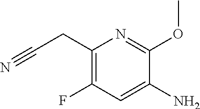

Step 1: To a stirred solution of (6-bromo-5-fluoro-2-methoxy-3-pyridyl)amine (2.5 g, 10.75 mmol) in N,N-dimethylacetamide (25 mL) was added sodium hydride (1.29 g, 32.24 mmol) portionwise (5×258 mg) at 0° C. After stirring at 0° C. for 30 min, 4-methoxybenzyl chloride (3.43 g, 2.99 mL, 21.49 mmol) was added and the reaction mixture and stirred for 30 min at room temperature before it was carefully quenched with sat. ammonium chloride solution, poured into water and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-10% to afford (6-bromo-5-fluoro-2-methoxy-3-pyridyl)-bis(p-anisyl)amine (4.93 g, 99%) as red viscous oil. 1H NMR (300 MHz, DMSO-d6) δ=7.22-7.12 (m, 5H), 6.91-6.81 (m, 4H), 4.24 (s, 4H), 3.95 (s, 3H), 3.71 (s, 6H)

Step 2: A solution of (6-bromo-5-fluoro-2-methoxy-3-pyridyl)-bis(p-anisyl)amine (1 g, 2.17 mmol) in toluene (20 mL) was cooled to −78° C. and 1.6 M n-butyllithium (1.75 g, 2.03 mL, 3.25 mmol) was added dropwise. The resulting dark blue solution was stirred at −78° C. for 30 min before N,N-dimethylformamide (396.11 mg, 419.17 μL, 5.42 mmol) was added. The stirring was continued at −78° C. for 30 min. before the reaction mixture was allowed to warm to room temperature. Methanol (4 mL) followed by sodium borohydride (82 mg, 2.17 mmol) were added. After 15 min the reaction mixture was quenched with sat. ammonium chloride solution and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-50% to afford [5-[bis(p-anisyl)amino]-3-fluoro-6-methoxy-2-pyridyl]methanol (297 mg, 32%) as light yellow viscous oil. MS (ESI): m/z=413.3 [M+H]⁺

Step 3: To a stirred solution of [5-[bis(p-anisyl)amino]-3-fluoro-6-methoxy-2-pyridyl]methanol (144 mg, 0.342 mmol) in dichloromethane (1.5 mL) was added thionyl dichloride (81.41 mg, 49.64 μL, 0.684 mmol) dropwise at room temperature. The reaction mixture was stirred at room temperature for 1 h before it was concentrated in vacuo to afford [6-(chloromethyl)-5-fluoro-2-methoxy-3-pyridyl]-bis(p-anisyl)amine (152 mg, 103%) as light brown foam, which was directly used in the next step without further purification.

Step 4: To a stirred solution of [6-(chloromethyl)-5-fluoro-2-methoxy-3-pyridyl]-bis(p-anisyl)amine (152 mg, 0.353 mmol) in dichloromethane (700 μL) was added tetrabutylammonium bromide (11.49 mg, 0.035 mmol) followed by a solution of sodium cyanide (21.39 mg, 0.423 mmol) in water (130 μL). The reaction mixture was stirred at room temperature for 15 h. Sodium cyanide (21.39 mg, 0.423 mmol) was added again and the stirring was continued at room temperature for another 15 h before the reaction mixture was diluted with dichloromethane and washed twice with water. The organic layers were washed twice with brine, dried over anhydrous sodium sulfate, filtered and concentrated in vacuo. The crude material was purified by flash chromatography over silica gel using a gradient ethyl acetate/heptane 0-30% to afford 2-[5-[bis(p-anisyl)amino]-3-fluoro-6-methoxy-2-pyridyl]acetonitrile (87 mg, 58%) as colorless viscous oil. MS (ESI): m/z=422.3 [M+H]⁺

Step 5: A solution of 2-[5-[bis(p-anisyl)amino]-3-fluoro-6-methoxy-2-pyridyl] acetonitrile (85 mg, 0.202 mmol) in dichloromethane (400 μL) was cooled to 0° C. and trifluoroacetic acid (1.38 g, 926.64 μL, 12.1 mmol) was added. The reaction mixture was stirred at 0° C. for 20 min and at room temperature for 4 h before it was poured into sat. NaHCO₃ and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-40% to afford 2-(5-amino-3-fluoro-6-methoxy-2-pyridyl) acetonitrile (31 mg, 84%) as light yellow solid. MS (ESI): m/z=182.1 [M+H]⁺

### Intermediate A4:5-(difluoromethoxy)-3-fluoro-6-methoxy-pyridin-2-amine

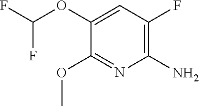

Step 1: To a stirred solution of (5-bromo-3,6-difluoro-2-pyridyl)amine (2.45 g, 11.74 mmol) in N,N-dimethylacetamide (25 mL) was added sodium hydride (1.41 g, 35.21 mmol) portionwise (5×282 mg) at 0° C. After stirring at 0° C. for 30 min 4-methoxybenzyl chloride (3.75 g, 3.26 mL, 23.47 mmol) was added and the reaction mixture was allowed to stir for another 30 min at room temperature before it was carefully quenched with sat. ammonium chloride solution, poured into water and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-10% to afford (5-bromo-3,6-difluoro-2-pyridyl)-bis(p-anisyl)amine (4.9 g, 92%) as light yellow viscous oil. MS (ESI): m/z=448.8 [M+H]⁺

Step 2: To a solution of [1,1′-bis(diphenylphosphino) ferrocene]palladium (II) chloride (1.27 g, 1.56 mmol), potassium acetate (2.92 mL, 46.74 mmol) and 5-bromo-3,6-difluoro-N,N-bis[(4-methoxyphenyl)methyl]pyridin-2-amine (7.0 g, 15.58 mmol) in 1,4-dioxane (70 mL) was added bis(pinacolato)diboron (5.93 g, 23.37 mmol) under nitrogen atmosphere. The mixture was stirred at 100° C. for 16 h before it was filtered, diluted with water and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/petroleum ether 0-15% to afford 3,6-difluoro-N,N-bis[(4-methoxyphenyl)methyl]-5-(4,4,5,5-tetramethyl-1,3,2 dioxaborolan-2-yl)pyridin-2-amine (4.2 g, 46%) as off-white solid. MS (ESI): m/z=497.0 [M+H]⁺

Step 3: To a solution of 3,6-difluoro-N,N-bis[(4-methoxyphenyl)methyl]-5-(4,4,5,5-tetramethyl-1,3,2-dioxaborolan-2-yl)pyridin-2-amine (3.70 g, 7.45 mmol) in THF (45 mL) at 0° C. hydrogen peroxide (16.9 g, 149.09 mmol) was added carefully. The reaction mixture was stirred at 0° C. for 15 min and at room temperature for additional 2 h before it was poured into cold 0.1 N aq. sodium thiosulfate (150 mL) and extracted twice with ethyl acetate. The organic layers were washed twice with brine, dried over anhydrous sodium sulfate, filtered and concentrated in vacuo (temp. of water bath: 20° C., do not evaporate to dryness) to afford the crude material 6-[bis[(4-methoxyphenyl)methyl]amino]-2,5-difluoro-pyridin-3-ol (3.30 g, 114%). The crude material was used directly as such for the next step without further purification. MS (ESI): m/z=387.2 [M+H]^{+Step} 4: To a solution of 6-[bis[(4-methoxyphenyl)methyl]amino]-2,5-difluoro-pyridin-3-ol (3.30 g, 8.54 mmol) in acetonitrile (35 mL) at 0° C. was added aqueous potassium hydroxide solution (34.16 mL, 170.81 mmol) dropwise, followed by addition of diethyl (bromodifluoromethyl)phosphonate (4.56 g, 17.08 mmol) in acetonitrile (10 mL). The resulting reaction mixture was stirred at 0° C. for 1 h before it was poured into water (200 mL) and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/petroleum ether 0-20% to afford 5-(difluoromethoxy)-3,6-difluoro-N,N-bis[(4-methoxyphenyl)methyl]pyridin-2-amine (3.31 g, 88%) as light yellow gum. 1H NMR (400 MHz, CHLOROFORM-d) 8 ppm 3.81 (s, 6H) 4.59 (s, 4H) 6.27-6.66 (m, 1H) 6.85-6.89 (m, 4H) 7.19 (d, J=8.56 Hz, 4H) 7.27-7.33 (m, 1H)

Step 5: A solution of 5-(difluoromethoxy)-3,6-difluoro-N,N-bis[(4-methoxyphenyl)methyl]pyridin-2-amine (3.30 g, 7.56 mmol) in dichloromethane (6 mL) and trifluoroacetic acid (31.46 mL, 408.34 mmol) was stirred at 0° C. for 1 h before it was poured into sat. NaHCO3 (200 mL) solution and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/petroleum ether 0-40% to afford 5-(difluoromethoxy)-3,6-difluoro-pyridin-2-amine (1.70 g, 114%) as light yellow oil. MS (ESI): m/z=196.7 [M+H]⁺

Step 6: To a solution of 5-(difluoromethoxy)-3,6-difluoro-pyridin-2-amine (1.70 g, 8.67 mmol) in THF (40 mL) was added sodium methoxide (468 mg, 8.67 mmol, 1 eq) at room temperature. The reaction mixture was stirred at 60° C. for 6 h before it was poured into water (40 mL) and extracted with twice with ethyl acetate. The organic layers were washed twice with brine, dried over anhydrous sodium sulfate, filtered and concentrated in vacuo. The crude material was purified twice by flash chromatography over silica gel using a gradient ethyl acetate/petroleum ether 0-30% to afford 5-(difluoromethoxy)-3-fluoro-6-methoxy-pyridin-2-amine (850 mg, 45%) as light yellow oil. MS (ESI): m/z=208.7 [M+H]⁺

### Intermediate A6:5-(2,2-difluoroethyl)-3-fluoro-6-methoxy-pyridin-2-amine

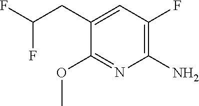

Step 1: A mixture of 5-bromo-3,6-difluoropyridin-2-amine (1.02 g, 4.88 mmol) and sodium methoxide (833 mg, 14.6 mmol) in THF (15 ml) was heated at 100° C. for 15 h before it was poured into sat. ammonium chloride solution. pH was adjusted to ca. 7 with aqueous 1 N HCl and the reaction mixture was extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-20% to afford 5-bromo-3-fluoro-6-methoxypyridin-2-amine (893 mg, 83%) as a light brown solid. MS (ESI): m/z=221.0 [M+H]⁺

Step 2: To a stirred solution of 5-bromo-3-fluoro-6-methoxypyridin-2-amine (200 mg, 0.905 mmol) in N,N-dimethylacetamide (3 mL) was added sodium hydride (108.57 mg, 2.71 mmol) at 0° C. After stirring at 0° C. for 20 min, 4-methoxybenzyl chloride (289.2 mg, 251.48 μL, 1.81 mmol) was added. The reaction mixture was allowed to warm to room temperature and stirred for 30 min before it was carefully quenched with sat. ammonium chloride solution, poured into water and extracted twice with ethyl acetate. The combined organic layers were washed twice with brine, dried over anhydrous sodium sulfate, filtered and concentrated in vacuo. The crude material was purified by flash chromatography over silica gel using a gradient ethyl acetate/heptane 0-20% to afford (5-bromo-3-fluoro-6-methoxy-2-pyridyl)-bis(p-anisyl)amine (270 mg, 64%) as light yellow viscous oil. MS (ESI): m/z=463.2 [M+H]⁺

Step 3: To a solution of (5-bromo-3-fluoro-6-methoxy-2-pyridyl)-bis(p-anisyl)amine (1.27 g, 2.75 mmol) in tetrahydrofuran (80 mL) at 0° C. was added isopropylmagnesium chloride-lithium chloride complex (1.3M in THF, 8.47 mL, 11.01 mmol). The reaction mixture was stirred 3 h at room temperature before it was cooled to 0° C. and N,N-dimethylformamide (1.61 g, 1.71 mL, 22.02 mmol) was added. After 30 min at room temperature the reaction mixture was cooled to 0° C. and quenched with aqueous potassium bisulfate solution (1 N, 30 ml), extracted with water and ethyl acetate. The combined organic layers were washed twice with brine, dried over anhydrous sodium sulfate, filtered and concentrated in vacuo. The crude material was purified by flash chromatography over silica gel using a gradient ethyl acetate/heptane 0-30% to afford the desired product (901 mg, 79%) as a colorless oil. MS (ESI): m/z=411.3 [M+H]⁺

Step 4: To a solution of 6-[bis(p-anisyl)amino]-5-fluoro-2-methoxy-nicotinaldehyde (933 mg, 2.16 mmol) in N,N-dimethylformamide (30 mL) was added triphenylphosphine (1.42 g, 5.4 mmol) and the reaction mixture was heated to 100° C. Sodium chlorodifluoroacetate (823.1 mg, 5.4 mmol) was added to the reaction mixture in 5 portions of ~165 mg within 10 min interval. The reaction mixture was allowed to cool down to room temperature, quenched with water and extracted twice with ethyl acetate. The combined organic layers were washed twice with brine, dried over anhydrous sodium sulfate, filtered and concentrated in vacuo. The crude material was purified by flash chromatography over silica gel using a gradient ethyl acetate/heptane 0-20% to afford the desired product (844 mg, 84%) as a light yellow oil. MS (ESI): m/z-445.3 [M+H]^{+Step} 5: To a solution of [5-(2,2-difluorovinyl)-3-fluoro-6-methoxy-2-pyridyl]-bis(p-anisyl)amine (836 mg, 1.88 mmol) was dissolved in methanol (50 mL) was added palladium hydroxide on carbon (264.15 mg, 0.094 mmol, 0.050 eq) and the reaction mixture was stirred under hydrogen balloon pressure at room temperature. After the reaction was completed, the reaction mixture was filtered through dicalite and washed with ethyl acetate. The combined organic layers were concentrated on vacuo and dried to afford 5-(2,2-difluoroethyl)-3-fluoro-6-methoxy-pyridin-2-amine (390 mg, 80%) as a light brown oil. MS (ESI): m/z=207.1 [M+H]⁺

### Intermediate A7:6-(2,2-difluoroethyl)-5-fluoro-2-methoxy-pyridin-3-amine

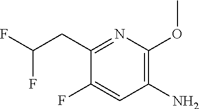

Step 1: To a stirred solution of (6-bromo-5-fluoro-2-methoxy-3-pyridyl)amine (2.5 g, 10.75 mmol) in N,N-dimethylacetamide (25 mL) was added sodium hydride (1.29 g, 32.24 mmol) portionwise (5×258 mg) at 0° C. After stirring at 0° C. for 30 min, 4-methoxybenzyl chloride (3.43 g, 2.99 mL, 21.49 mmol) was added and the reaction mixture and stirred for 30 min at room temperature before it was carefully quenched with sat. ammonium chloride solution, poured into water and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-10% to afford (6-bromo-5-fluoro-2-methoxy-3-pyridyl)-bis(p-anisyl)amine (4.93 g, 99%) as red viscous oil. 1H NMR (300 MHz, DMSO-d6) δ=7.22-7.12 (m, 5H), 6.91-6.81 (m, 4H), 4.24 (s, 4H), 3.95 (s, 3H), 3.71 (s, 6H)

Step 2: To a solution of (6-bromo-5-fluoro-2-methoxy-3-pyridyl)-bis(p-anisyl)amine (1.33 g, 2.88 mmol) in tetrahydrofuran (25 mL) at −78° C. was added n-butyllithium (1.6 M in hexane, 1.8 mL, 2.88 mmol) slowly. After 15 min at −78° C. N,N-dimethylformamide (273.94 mg, 290.19 μL, 3.75 mmol) was added and the mixture was stirred for additional 5 min at −78° C. before it was allowed to warm up to −20° C. When LCMS indicated full conversion, the reaction was quenched with sat. ammonium chloride solution at −78° C., warmed to room temperature and extracted twice with ethyl acetate. The organic layers were washed twice with brine, dried over anhydrous sodium sulfate, filtered and concentrated in vacuo. The crude material was purified by flash chromatography over silica gel using a gradient ethyl acetate/heptane 0-30% to afford the desired product (807 mg, 65%) as a yellow foam. MS (ESI): m/z=411.3 [M+H]⁺

Step 3: To a solution of 5-[bis(p-anisyl)amino]-3-fluoro-6-methoxy-picolinaldehyde (805 mg, 1.96 mmol) in N,N-dimethylformamide (30 mL) was added triphenylphosphine (1.29 g, 4.9 mmol) and the reaction mixture was heated to 100° C. Sodium chlorodifluoroacetate (747.55 mg, 4.9 mmol) was added to the reaction mixture in 5 portions of ~150 mg within 10 min interval. The reaction mixture was allowed to cool down to room temperature, quenched with water and extracted twice with ethyl acetate. The combined organic layers were washed twice with brine, dried over anhydrous sodium sulfate, filtered and concentrated in vacuo. The crude material was purified by flash chromatography over silica gel using a gradient ethyl acetate/heptane 0-20% to afford the desired product (818 mg, 90%) as a colorless oil. MS (ESI): m/z=445.3 [M+H]^{+Step} 5: To a solution of [6-(2,2-difluorovinyl)-5-fluoro-2-methoxy-3-pyridyl]-bis(p-anisyl)amine (812 mg, 1.74 mmol) was dissolved in methanol (50 mL) was added palladium hydroxide on carbon (243.73 mg, 0.087 mmol, 0.050 eq) and the reaction mixture was stirred under hydrogen balloon pressure at room temperature. After the reaction was completed, the reaction mixture was filtered through dicalite and washed with ethyl acetate. The combined organic layers were concentrated on vacuo and dried to afford 6-(2,2-difluoroethyl)-5-fluoro-2-methoxy-pyridin-3-amine (355 mg, 89%) as a light brown oil. MS (ESI): m/z=207.1 [M+H]⁺

### Intermediate A10:3-fluoro-5-(2-fluoroethoxy)-6-methoxy-pyridin-2-amine

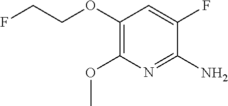

A solution of 3,6-difluoro-5-(2-fluoroethoxy)pyridin-2-amine (29 mg, 0.151 mmol, described in WO2019243398) and sodium methylate (25.75 mg, 0.453 mmol) in methanol (1 mL) was heated to 100° C. and stirred for 15 h. Sodium methylate (12.87 mg, 0.226 mmol) was added and the stirring was continued at 100° C. for 20 h. The reaction mixture was cooled to room temperature and concentrated in vacuo. The residue was poured into water and extracted twice with ethyl acetate. The combined organic layers were washed twice with brine, dried over anhydrous sodium sulfate, filtered and concentrated in vacuo to afford [3-fluoro-5-(2-fluoroethoxy)-6-methoxy-2-pyridyl]amine (26 mg, 81%) as brown gum. MS (ESI): m/z=205.1 [M+H]⁺

### Intermediate A13:6-(3,3-difluoropropyl)-5-fluoro-2-methoxy-pyridin-3-amine

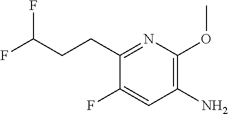

Step 1: To a solution of (6-bromo-5-fluoro-2-methoxy-3-pyridyl)amine (1.25 g, 5.66 mmol) in dichloromethane (30 mL) at 0° C. were added N,N-diisopropylethylamine (877.08 mg, 1.19 mL, 6.79 mmol) and 4-dimethylaminopyridine (34.55 mg, 0.283 mmol) and a solution of di-tert-butyl dicarbonate (1.36 g, 1.44 mL, 6.22 mmol) in dichloromethane (15 mL). The reaction mixture was stirred at room temperature before additional di-tert-butyl dicarbonate (1.23 g, 1.31 mL, 5.66 mmol) was added after 3 h and the stirring was continued at room temperature overnight. The reaction mixture was poured into water and extracted with dichloromethane. The combined organic layers were washed twice with brine, dried over anhydrous sodium sulfate, filtered and concentrated in vacuo. The crude material was purified by flash chromatography over silica gel using a gradient ethyl acetate/heptane 0-30% to afford the desired product (1.2 g, 40%) as a white semisolid. MS (ESI): m/z=445.06 [M+H+Na]⁺

Step 2: To a solution of N-(6-bromo-5-fluoro-2-methoxy-3-pyridyl)-N-tert-butoxycarbonyl-carbamic acid tert-butyl ester (1.2 g, 2.85 mmol), (E)-3-(4,4,5,5-tetramethyl-1,3,2-dioxaborolan-2-yl) acrylic acid ethyl ester (837.21 mg, 828.92 μL, 3.7 mmol) and cesium carbonate (2.78 g, 8.55 mmol) in 1,4-dioxane (6 mL), water (1 mL) was added 1,1′-bis(diphenylphosphino) ferrocene-palladium (II) dichloride dichloromethane complex (235.5 mg, 0.285 mmol). The reaction mixture was stirred at 100° C. for 2 h. The residue was poured into water and extracted twice with ethyl acetate. The combined organic layers were washed twice with brine, dried over anhydrous sodium sulfate, filtered and concentrated in vacuo. The crude material was adsorbed on ISOLUTE HM-N and purified by flash chromatography over silica gel using a gradient ethyl acetate/heptane 0-35% to afford the desired product (747 mg, 56%) as a colorless oil. MS (ESI): m/z=441.3 [M+H]^{+Step} 3: (E)-3-[5-[bis(tert-butoxycarbonyl)amino]-3-fluoro-6-methoxy-2-pyridyl] acrylic acid ethyl ester (740 mg, 1.68 mmol) was dissolved in ethyl acetate (30 mL) under argon. Palladium on activated charcoal 10% (178.79 mg, 0.168 mmol) was added and the flask was purged and backfilled with argon. The reaction mixture was stirred under hydrogen atmosphere at room temperature for 2 h before it was filtered on decalite and washed with ethyl acetate. Filtrate was concentrated in vacuo to afford the desired product (744 mg, 95%) as a colorless oil. MS (ESI): m/z=443.3 [M+H]⁺

Step 4: In a 50 ml three-necked flask, 3-[5-[bis(tert-butoxycarbonyl)amino]-3-fluoro-6-methoxy-2-pyridyl]propionic acid ethyl ester (667 mg, 1.51 mmol) was dissolved in tetrahydrofuran (10 mL) and the colorless solution was cooled to −78° C. DIBAL-H (1M in THF, 1.51 mL, 1.51 mmol) was added at −78° C. and the reaction mixture was stirred at this temperature for 1 h. Additional DIBAL-H (1M in THF, 1.51 mL, 1.51 mmol) was added at −78° C. and the reaction mixture was stirred at room temperature. After 2 h the reaction mixture was cooled to −78° C., water (120 μL) were added followed by 15% aq. NaOH (120 μL) and additional water (300 μL). The reaction mixture was warmed to room temperature and stirred 30 min then sodium sulfate was added. The reaction mixture was filtered over celite and concentrated in vacuo. The crude material was adsorbed on ISOLUTE HM-N and purified by flash chromatography over silica gel using a gradient ethyl acetate/heptane 0-50% to afford the desired product (423 mg, 56%) as a colorless oil. MS (ESI): m/z=401.3 [M+H]⁺

Step 5: N-tert-butoxycarbonyl-N-[5-fluoro-6-(3-hydroxypropyl)-2-methoxy-3-pyridyl]carbamic acid tert-butyl ester (416 mg, 1.04 mmol) was dissolved in dichloromethane (10 mL) and the solution was cooled to 0° C. 1,1,1-tris (acetyloxy)-1,1-dihydro-1,2-benzodioxol-3-(1H)-one (Dess-Martin periodinane) (528.75 mg, 1.25 mmol) was added, the ice bath was removed and the reaction mixture was stirred 45 min at room temperature. The reaction mixture was poured into saturated NaHCO₃ solution and extracted with dichloromethane. The combined organic layers were washed twice with brine, dried over anhydrous sodium sulfate, filtered and concentrated in vacuo. The crude material was adsorbed on ISOLUTE HM-N and purified by flash chromatography over silica gel using a gradient ethyl acetate/heptane 0-50% to afford the desired product (359 mg, 84%) as a colorless oil. MS (ESI): m/z=399.3 [M+H]⁺

Step 6: N-tert-butoxycarbonyl-N-[5-fluoro-6-(3-ketopropyl)-2-methoxy-3-pyridyl]carbamic acid tert-butyl ester (355 mg, 0.891 mmol, 1 eq) was dissolved in dichloromethane (10 mL) and the solution was cooled to 0° C. [bis(2-methoxyethyl)amino] sulfur trifluoride (DeoxoFluor 2.7M in toluene, 792. mg, 660 μL, 1.78 mmol) was added dropwise at 0° C. and the reaction mixture was stirred 30 min at 0° C. and at room temperature. After 2 h the reaction mixture was poured into saturated NaHCO₃/ice mixture and stirred for 10 min. The layers were separated and the aqueous layer was extracted with dichloromethane. The combined organic layers were washed twice with brine, dried over anhydrous sodium sulfate, filtered and concentrated in vacuo. The crude material was adsorbed on ISOLUTE HM-N and purified by flash chromatography over silica gel using a gradient ethyl acetate/heptane 0-30% to afford the desired product (110 mg, 11%) as a colorless oil. MS (ESI): m/z=421.3 [M+H]⁺

Step 7: A mixture of N-tert-butoxycarbonyl-N-[6-(3,3-difluoropropyl)-5-fluoro-2-methoxy-3-pyridyl]carbamic acid tert-butyl ester (107 mg, 0.255 mmol) and HCl (4M in dioxane, 1.53 g, 1.27 mL, 5.09 mmol) was stirred at room temperature. After 2.5 h the reaction mixture was poured into saturated NaHCO₃ solution and extracted with ethyl acetate. The combined organic layers were washed twice with brine, dried over anhydrous sodium sulfate, filtered and concentrated in vacuo. The crude material was adsorbed on ISOLUTE HM-N and purified by flash chromatography over silica gel using a gradient ethyl acetate/heptane 0-100% to afford the desired product (15 mg, 25%) as a colorless oil. MS (ESI): m/z=221.1 [M+H]⁺

### Intermediate A14:2,6-bis(difluoromethoxy)-5-fluoro-pyridin-3-amine

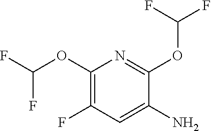 [SMILES: Nc1cc(F)c(OC(F)F)nc1OC(F)F]

Step 1: To a solution of 4-methoxybenzyl alcohol (3.54 g, 25.61 mmol) in THF (120 mL) was added sodiumbis(trimethylsilyl)amide (25.61 mL, 25.61 mmol) at −78° C. and the resulting reaction mixture was stirred for 30 min before it was added to the solution of 2,3,6-trifluoro-5-nitro-pyridine (4.8 g, 26.96 mmol) in THF (50 mL) at −78° C. After 1 h at −78° C. the reaction mixture was quenched with aq. NH4Cl (125 mL) and extracted with ethyl acetate. The combined organic layers were washed twice with brine, dried over anhydrous sodium sulfate, filtered and concentrated in vacuo. The residue was purified by flash chromatography over silica gel using a gradient ethyl acetate/petroleum ether 0-10% to afford 2,5-difluoro-6-[(4-methoxyphenyl) methoxy]-3-nitro-pyridine (3.79 g, 47%) as yellow solid. 1H NMR (400 MHz, DMSO-d6) δ=8.77 (dd, J=7.5, 8.8 Hz, 1H), 7.45 (d, J=8.7 Hz, 2H), 7.03-6.91 (m, 2H), 5.44 (s, 2H), 3.76 (s, 3H).

Step 2: To a solution of 2,5-difluoro-6-[(4-methoxyphenyl) methoxy]-3-nitro-pyridine (3.98 g, 13.44 mmol) in THF (200 mL) was added sodium methoxide (2.69 g, 13.44 mmol). The mixture was stirred at −20° C. for 2 h. The mixture was quenched with aq. NH4Cl (200 mL and extracted with ethyl acetate. The combined organic layers were washed twice with brine, dried over anhydrous sodium sulfate, filtered and concentrated in vacuo to give a crude product 5-fluoro-2-methoxy-6-[(4-methoxyphenyl) methoxy]-3-nitro-pyridine (4.04 g, 97%) as a yellow solid. 1H NMR (400 MHz, DMSO-d6) δ=8.52 (d, J=9.4 Hz, 1H), 7.45 (d, J=8.4 Hz, 2H), 6.97 (d, J=8.7 Hz, 2H), 5.52 (s, 2H), 4.08 (s, 3H), 3.76 (s, 3H).

Step 3: To a solution of 5-fluoro-2-methoxy-6-[(4-methoxyphenyl) methoxy]-3-nitro-pyridine (5.88 g, 19.07 mmol) in dichloromethane (59 mL) was added trifluoroacetic acid (59.0 mL, 765.81 mmol) at 25° C., the reaction mixture was stirred at 25° C. for 12 h. The reaction mixture was concentrated under reduced pressure to give a residue and purified by flash chromatography over silica gel using a gradient ethyl acetate/petroleum ether 0-50% to afford 3-fluoro-6-methoxy-5-nitro-pyridin-2-ol (5.4 g, 78%) as brown solid. 1H NMR (400 MHz, DMSO-d6) δ=8.41 (d, J=9.5 Hz, 1H), 3.97 (s, 3H).

Step 4: To a solution of 3-fluoro-6-methoxy-5-nitro-pyridin-2-ol (1.0 g, 5.32 mmol) in acetonitrile (20 mL) were added a solution of KOH (2.98 g, 53.16 mmol) in water (3.5 mL) and diethyl (bromodifluoromethyl)phosphonate (8.52 g, 31.9 mmol) at 40° C. The reaction mixture was stirred at 40° C. for 4 h before it was diluted with water and extracted with ethyl acetate. The combined organic layers were washed twice with brine, dried over anhydrous sodium sulfate, filtered and concentrated in vacuo. The residue was purified by flash chromatography over silica gel using a gradient ethyl acetate/petroleum ether 0-3% to afford 2-(difluoromethoxy)-3-fluoro-6-methoxy-5-nitro-pyridine (580 mg, 45%) as a yellow oil. 1H NMR (400 MHz, DMSO-d6) δ=8.79 (d, J=9.0 Hz, 1H), 8.10-7.70 (m, 1H), 4.04 (s, 3H).

Step 5: To a mixture of 2-(difluoromethoxy)-3-fluoro-6-methoxy-5-nitro-pyridine (1.7 g, 7.14 mmol) in dichloromethane (24 mL) was added BBr3 (3.34 mL, 35.7 mmol) at 0° C. and the reaction mixture was stirred at 20° C. for 1 h. The reaction mixture was poured into water and extracted with ethyl acetate. The combined organic layers were washed twice with brine, dried over anhydrous sodium sulfate, filtered and concentrated in vacuo. The residue was purified by flash chromatography over silica gel using a gradient ethyl acetate/petroleum ether 0-50% to afford 6-(difluoromethoxy)-5-fluoro-3-nitro-pyridin-2-ol (1.48 g, 92%) as yellow oil. 1H NMR (400 MHz, CHLOROFORM-d) 8 ppm 7.31-7.69 (m, 1H) 8.33 (d, J=7.70 Hz, 1H) 11.35 (br s, 1H)

Step 6: To a solution of 6-(difluoromethoxy)-5-fluoro-3-nitro-pyridin-2-ol (1.48 g, 6.6 mmol) in acetonitrile (20 mL) and KOH (3705.78 mg, 66.04 mmol) in water (5 mL) was added diethyl (bromodifluoromethyl)phosphonate (10580.79 mg, 39.63 mmol) at 40° C. After 1 h the reaction mixture was cooled to room temperature and extracted with dichloromethane. The combined organic layers were washed twice with brine, dried over anhydrous sodium sulfate, filtered and concentrated in vacuo. The residue was purified by flash chromatography over silica gel using a gradient ethyl acetate/petroleum ether 0-20% to afford 2,6-bis(difluoromethoxy)-3-fluoro-5-nitro-pyridine (2.38 g, 46%) as light yellow oil. 1H NMR (400 MHz, DMSO-d6) δ ppm 7.64-8.12 (m, 2H) 8.98 (d, J=8.93 Hz, 1H)

Step 7: To a mixture of 2,6-bis(difluoromethoxy)-3-fluoro-5-nitro-pyridine (2.38 g, 3.04 mmol) in ethanol (24 mL) and water (6 mL) were added NH4Cl (805.34 mg, 15.2 mmol) and Fe (850.93 mg, 15.2 mmol) at 25° C. The reaction mixture was stirred at 80° C. for 2 h. before it was cooled to room temperature and extracted with ethyl acetate. The combined organic layers were washed twice with brine, dried over anhydrous sodium sulfate, filtered and concentrated in vacuo The residue was purified by flash chromatography over silica gel using a gradient ethyl acetate/petroleum ether 0-30% to afford 2,6-bis(difluoromethoxy)-5-fluoro-pyridin-3-amine (639.5 mg) as light brown oil. MS (ESI): m/z=244.7 [M+H]⁺

### Intermediate A15:5-cyclopropyl-3-fluoro-6-methoxy-pyridin-2-amine

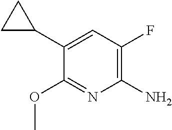

Step 1: To a stirred solution of 3,6-difluoropyridin-2-amine (1.00 g, 7.69 mmol) in acetonitrile (15 ml) was added a suspension of N-bromosuccinimide (1.73 g, 9.61 mmol) in acetonitrile (10 ml). The reaction mixture was stirred at room temperature for 1 h before it was diluted with water and extracted with ethyl acetate. The combined organic layers were washed twice with brine, dried over anhydrous sodium sulfate, filtered and concentrated in vacuo (don't evaporate to dryness due to formation of impurities!). The residue was purified by flash chromatography over silica gel using a gradient ethyl acetate/heptane 0-20% to afford 5-bromo-3,6-difluoropyridin-2-amine (1.44 g, 89%) as an orange solid. MS (ESI) m/z: 209.0 [M+H]⁺

Step 2: A mixture of 5-bromo-3,6-difluoropyridin-2-amine (1.02 g, 4.88 mmol) and sodium methoxide (833 mg, 14.6 mmol) in THF (15 ml) was heated to 100° C. After 15 h the reaction mixture was cooled to room temperature and poured into sat. NH4Cl solution. The pH was adjusted to ca. 7 with 1 N HCl and the mixture was extracted twice with ethyl acetate. The combined organic layers were washed twice with brine, dried over anhydrous sodium sulfate, filtered and concentrated in vacuo. The crude material was purified by flash chromatography on silica gel using a gradient ethyl acetate/heptane 0-20% to afford 5-bromo-3-fluoro-6-methoxypyridin-2-amine (893 mg, 82%) as a light brown solid. MS (ESI) m/z: 221.0 [M+H]⁺

Step 3: A mixture of (5-bromo-3-fluoro-6-methoxy-2-pyridyl)amine (100 mg, 0.452 mmol), cyclopropylboronic acid (77.73 mg, 0.905 mmol), tricyclohexylphosphine (13.08 mg, 0.045 mmol), palladiumacetate (5.08 mg, 0.023 mmol) and potassium phosphate tribasic (346.52 mg, 1.58 mmol) in toluene (1.8 mL) and water (100 μL) was heated to 100° C. After 6 h the reaction mixture was poured into water and extracted twice with ethyl acetate. The combined organic layers were washed twice with brine, dried over anhydrous sodium sulfate, filtered and concentrated in vacuo. The crude material was purified by flash chromatography on silica gel using a gradient ethyl acetate/heptane 0-20% to afford (5-cyclopropyl-3-fluoro-6-methoxy-2-pyridyl)amine (55 mg, 65%) as orange oil. MS (ESI) m/z: 183.1 [M+H]⁺

### Intermediate A16: (5-bromo-4,6-dimethoxy-pyrimidin-2-yl)amine

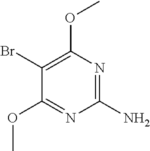

To a stirred solution of (4,6-dimethoxypyrimidin-2-yl)amine (100 mg, 0.632 mmol, CAS: 36315 01-2) in acetonitrile (2 mL) was added N-bromosuccinimide (147.65 mg, 0.821 mmol) at room temperature. The reaction mixture was stirred at room temperature and the obtained light yellow solution was diluted with ethyl acetate and washed with water. The organic layer was dried over sodium sulfate, filtered and concentrated in vacuo. The residue was purified by flash chromatography over silica gel using a gradient ethylacetate/heptane 0-50% to provide the title compound as an off-white solid (116 mg, 78%). MS (ESI): m/z=234.1 [M+H]⁺

### Intermediate A17: (4,6-dimethoxy-5-methyl-pyrimidin-2-yl)amine

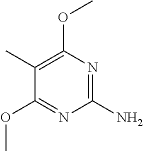

A suspension of (4,6-dichloro-5-methyl-pyrimidin-2-yl)amine (100 mg, 0.551 mmol, CAS: 6343-68-6)) and sodium methylate (156.53 mg, 2.75 mmol) in methanol (3 mL) was heated to 95° C. and stirred for 15 h. Sodium methylate (78.26 mg, 1.38 mmol) was added again. The reaction mixture was heated to 100° C. and stirred for 15 h. The reaction mixture was concentrated in vacuo. The residue was poured into water and extracted twice with ethyl acetate. The organic layers were dried over sodium sulfate, filtered and concentrated in vacuo to afford (4,6-dimethoxy-5-methyl-pyrimidin-2-yl)amine (91 mg, 97%) as white solid. MS (ESI) m/z: 169.8 [M+H]⁺

### Intermediate A20:5-fluoro-2-methoxy-6-(oxetan-3-yl)pyridin-3-amine

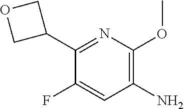

Step 1: To a stirred solution of (5-fluoro-2-methoxy-3-pyridyl)amine (900 mg, 6.33 mmol) in acetonitrile (30 mL) was added a solution of N-bromosuccinimide (1.13 g, 6.33 mmol) in acetonitrile (10 mL) dropwise at room temperature. The reaction mixture was stirred at room temperature for 30 min before it was diluted with water and extracted with ethyl acetate. The combined organic layers were washed twice with brine, dried over anhydrous sodium sulfate, filtered and concentrated in vacuo. The crude material was purified by flash chromatography on silica gel using a gradient ethyl acetate/heptane 0-30% to afford the desired product (1.25 g, 87%) as a dark red solid. MS (ESI): m/z=221.0 [M+H]⁺

Step 2: A stock solution of the Ir-Catalyst (6.9 mg/mL in dichloromethane) and of NiCl2*glyme/dtbbpy (2.7 mg Ni—Co-Cat/mL and 2.2 mg ligand/mL in dimethoxyethane) were prepared.

The corresponding volume of the Ir-Catalyst solution for [4,4′-bis(1,1-dimethylethyl)-2,2′-bipyridine-n1,n1′]bis [3,5-difluoro-2-[5-(trifluoromethyl)-2-pyridinyl-n] phenyl-c] iridium (III) hexafluorophosphate-(Ir[df(cf3)ppy]2(dtbpy))pf6 (7.61 mg, 0.007 mmol, 0.010 eq) was added to the reaction vial and the solvent was evaporated. Then sodium carbonate (142.51 mg, 1.36 mmol, 2 eq), (6-bromo-5-fluoro-2-methoxy-3-pyridyl)amine (150 mg, 0.679 mmol, 1 eq), 3-bromooxetane (185.92 mg, 112.61 μL, 1.36 mmol, 2 eq) and tris (trimethylsilyl) silane (226.13 mg, 280.55 μL, 0.909 mmol, 1.34 eq) were added. Ethylene glycol dimethyl ether, extra dry (10.95 mL) was added and the reaction mixture was degassed by bubbling argon through the mixture for 2 min. The corresponding volume of the NiCl2*glyme/dtbbpy solution for nickel (II) chloride ethylene glycol dimethyl ether complex (745.56 μg, 0.003 mmol, 0.005 eq) and 4,4′-bis(tert-butyl)-2,2′-bipyridine (910.72 μg, 0.003 mmol, 0.005 eq) was added and the reaction was degassed again for 5 min. The vial was placed in the photoreactor and irradiated with 450 nm for 14 h (stirred at 900 rpm, LED 100% power). The reaction mixture was filtered and concentrated in vacuo. The residue was purified by column chromatography to afford [5-fluoro-2-methoxy-6-(oxetan-3-yl)-3-pyridyl]amine (47 mg, 35%) as off-white solid. (ESI): m/z=199.1 [M+H]⁺

### Intermediate A22:5-(2-fluoroethoxy)-4-methoxy-pyrimidin-2-amine

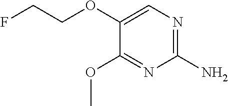

Step 1: A suspension of 5-bromo-2-chloro-4-methoxy-pyrimidine (1.02 g, 4.48 mmol, CAS: 57054-929), bis(p-anisyl)amine (1.29 g, 4.92 mmol) and n-ethyldiisopropylamine (858 μL, 4.92 mmol) in acetonitrile (20 mL) was heated at 70° C. for 2 days. The resulting solution was poured into a saturated aqueous sodium bicarbonate solution and extracted twice with ethylacetate. The organic layers were dried over sodium sulfate, filtered and dried in vacuo. The residue was purified by flash chromatography over silica gel using a gradient ethylacetate/heptane 0-20% to provide the title compound as a colorless viscous oil (998 mg, 50%). MS (ESI): m/z=446.2 [M+H]^{+Step} 2: A suspension of (5-bromo-4-methoxy-pyrimidin-2-yl)-bis(p-anisyl)amine (500 mg, 1.13 mmol), bis(pinacolato)diboron (354 mg, 1.35 mmol) and potassium acetate (335 mg, 3.38 mmol) in 1,4-dioxane (10 mL) was purged with argon for 5 min. dichloro[1,1′-bis(diphenylphosphino) ferrocene]palladium (II) dichloromethane adduct (91.9 mg, 0.113 mmol) was added. The reaction mixture was heated to 90° C. and stirred for 16 hours. The resulting dark suspension was poured into ethylacetate and washed once with sat. NaCl. The organic layer was dried over sodium sulfate, filtered and concentrated in vacuo. The residue was purified by column chromatography on silica gel using a gradient ethylacetate/heptane 0-30% to provide the title compound as a colorless viscous oil (157 mg, 29%). MS (ESI): m/z=492.4 [M+H]⁺

Step 3: A solution of [4-methoxy-5-(4,4,5,5-tetramethyl-1,3,2-dioxaborolan-2-yl)pyrimidin-2-yl]-bis(p-anisyl)amine (130 mg, 0.265 mmol) in tetrahydrofuran (2.5 mL) was cooled to 0° C. Hydrogen peroxide 35% (500 μL, 5.71 mmol) was added. The reaction mixture was stirred at 0° C. for 15 min, allowed to warm to room temperature and stirred for 3 hours. The reaction mixture was poured into cold 0.1 N sodiumsulfite solution and extracted twice with EtOAc. The organic layers were washed twice with brine, dried over sodiumsulfate, filtered and concentrated in vacuo to provide the title compound as a light yellow viscous oil (103 mg, 100%). MS (ESI): m/z=382.3 [M+H]⁺

Step 4: A suspension of 2-[bis(p-anisyl)amino]-4-methoxy-pyrimidin-5-ol (100 mg, 0.236 mmol), potassium carbonate (98.82 mg, 0.708 mmol) and 1-bromo-2-fluoroethane (61.14 mg, 35.75 μL, 0.472 mmol) in acetonitrile (2.5 mL) was stirred at room temperature for 15 min and at 80° C. for 6 hours. The reaction mixture was poured into water and extracted twice with EtOAc. The organic layers were dried over sodium sulfate, filtered and concentrated in vacuo. The residue was purified by flash chromatography on silica gel using a gradient ethylacetate/heptane 0-30% to provide the title compound as a colorless viscous oil (22 mg, 22%). MS (ESI): m/z=428.3 [M+H]^{+Step} 5: A solution of [5-(2-fluoroethoxy)-4-methoxy-pyrimidin-2-yl]-bis(p-anisyl)amine (87 mg, 0.204 mmol) in dichloromethane (500 μL) was cooled to 0° C. Trifluoroacetic acid (1.41 g, 944.56 uL, 12.21 mmol) was added. The reaction mixture was allowed to warm to room temperature and stirred for 16 hours and at 55° C. for two additional hours. The resulting purple solution was poured into a sat. aqueous sodium bicarbonate solution and extracted twice with EtOAc. The organic layers were dried over sodium sulfate, filtered and concentrated in vacuo. The residue was purified by flash chromatography on silica gel using a gradient ethylacetate/heptane 0-100% to provide the title compound as an off-white solid (27 mg, 71%). MS (ESI): m/z=188.1 [M+H]⁺

### Intermediate A23:5-(2,2-difluoroethoxy)-4,6-dimethoxy-pyrimidin-2-amine

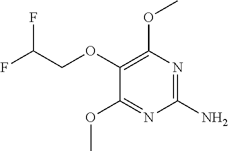 [SMILES: COc1nc(N)nc(OC)c1OCC(F)F]

### Step 1:5-bromo-4,6-dimethoxy-pyrimidin-2-amine

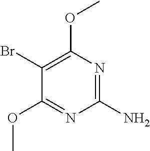

To a stirred solution of (4,6-dimethoxypyrimidin-2-yl)amine (7 g, 44.22 mmol, CAS: 36315-01-2) in acetonitrile (100 mL) was added a solution of N-bromosuccinimide (10.33 g, 57.48 mmol) in acetonitrile (100 mL) dropwise at room temperature. The reaction mixture was stirred at room temperature for 30 min. The resulting white suspension was diluted with ethyl acetate and washed with water. The organic layer was dried over sodium sulfate, filtered, diluted with heptane and concentrated in vacuo. The precipitate was filtered off and washed with heptane to provide the title compound as a white solid (9.26 g, 87% yield). MS (ESI) m/z=234.1 [M+H]⁺

### Step 2:5-bromo-4,6-dimethoxy-N,N-bis[(4-methoxyphenyl)methyl]pyrimidin-2-amine

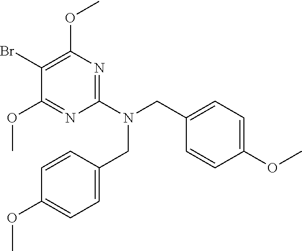 [SMILES: COc1ccc(CN(Cc2ccc(OC)cc2)c2nc(OC)c(Br)c(OC)n2)cc1]

A solution of 5-bromo-4,6-dimethoxy-pyrimidin-2-amine (517 mg, 2.21 mmol) in N,N-dimethylacetamide (9 mL) was cooled to 0° C. Sodium hydride (265.05 mg, 6.63 mmol) was added portionwise (3×88 mg). The stirring was continued at 0° C. for 30 min. 4-methoxybenzyl chloride (706. mg, 608.62 μL, 4.42 mmol) was added dropwise. The reaction mixture was allowed to warm to room temperature, stirred for 1 hour, carefully quenched with a saturated ammonium chloride solution, poured into water and extracted twice with ethyl acetate. The organic layers were dried over sodium sulfate, filtered and concentrated in vacuo. The residue was purified by flash chromatography over silica gel using a gradient ethylacetate/heptane 0-20% to provide the title compound as a white solid (1.11 g, 100% yield). MS (ESI) m/z=476.2 [M+H]⁺

### Step 3:2-[bis[(4-methoxyphenyl)methyl]amino]-4,6-dimethoxy-pyrimidin-5-ol

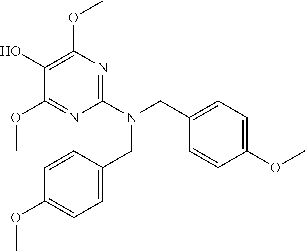 [SMILES: COc1ccc(CN(Cc2ccc(OC)cc2)c2nc(OC)c(O)c(OC)n2)cc1]

To a colorless solution of 5-bromo-4,6-dimethoxy-N,N-bis[(4-methoxyphenyl)methyl]-pyrimidin-2-amine (500 mg, 1 mmol) in tetrahydrofuran (3.5 mL) was added a 1.6 M n-butyllithium solution in hexanes (699.71 mg, 813.61 μL, 1.3 mmol) dropwise at −78° C. The resulting yellow solution was stirred at −78° C. for 30 min. Trimethyl borate (156.08 mg, 167.47 uL, 1.5 mmol) was added dropwise and the stirring was continued at −78° C. for 1.5 hours. The reaction mixture was allowed to warm to 0° C. and acetic acid (120.27 mg, 114.62 μL, 2 mmol) was added dropwise, followed by hydrogen peroxide 35% (145.98 mg, 131.51 μL, 1.5 mmol). The stirring was continued at 0° C. for 1.5 hours. The resulting pink suspension was poured into a 0.1 N sodium thiosulfate solution and extracted twice with ethyl acetate. The organic layers were washed with brine, dried over sodium sulfate, filtered and concentrated in vacuo. The residue was purified by flash chromatography over silica gel using a gradient ethylacetate/heptane 0-30% to provide the title compound as a light yellow viscous oil (194 mg, 47% yield). MS (ESI) m/z=412.3 [M+H]⁺

### Step 4:5-(2,2-difluoroethoxy)-4,6-dimethoxy-N,N-bis[(4-methoxyphenyl)methyl]pyrimidin-2-amine

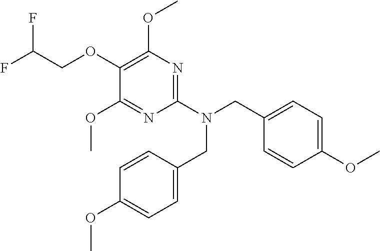 [SMILES: COc1ccc(CN(Cc2ccc(OC)cc2)c2nc(OC)c(OCC(F)F)c(OC)n2)cc1]

To a solution of 2-[bis[(4-methoxyphenyl)methyl]amino]-4,6-dimethoxy-pyrimidin-5-ol (100 mg, 0.214 mmol) in N,N-dimethylformamide (1.75 mL) was added potassium carbonate (88.68 g, 0.642 mmol) and 1,1-difluoro-2-iodoethane (123.16 mg, 56.5 μL, 0.642 mmol). The reaction mixture was stirred at 80° C. for 1.5 hours, cooled to room temperature, poured into water and extracted twice with ethyl acetate. The organic layers were dried over sodium sulfate, filtered and concentrated in vacuo. The residue was purified by flash chromatography over silica gel using a gradient ethylacetate/heptane 0-20% to provide the title compound as an off-white solid (92 mg, 85% yield). MS (ESI) m/z=476.2 [M+H]⁺

### Step 5:5-(2,2-difluoroethoxy)-4,6-dimethoxy-pyrimidin-2-amine

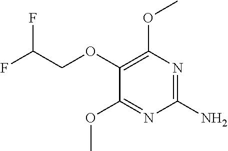 [SMILES: COc1nc(N)nc(OC)c1OCC(F)F]

To a stirred solution of 5-(2,2-difluoroethoxy)-4,6-dimethoxy-N,N-bis[(4-methoxyphenyl)methyl]pyrimidin-2-amine (92 mg, 0.193 mmol) in dichloromethane (0.340 mL) was added trifluoroacetic acid (1.34 g, 897.97 μL, 11.61 mmol) at 0° C. The reaction mixture was stirred at room temperature for 18 hours and at 50° C. for 4 hours. The resulting red solution was concentrated in vacuo, poured into sat. NaHCO₃ and extracted twice with ethyl acetate. The organic layers were dried over sodium sulfate, filtered and concentrated in vacuo. The residue was purified by flash chromatography over silica gel using a gradient ethylacetate/heptane 0-30% to provide the title compound as a light yellow solid (41 mg, 87% yield). MS (ESI) m/z=236.2 [M+H]⁺

### Intermediate A24:5-(2-fluoroethoxy)-4,6-dimethoxy-pyrimidin-2-amine

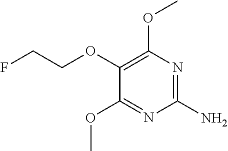

### Step 1:5-(2-fluoroethoxy)-4,6-dimethoxy-N,N-bis[(4-methoxyphenyl)methyl]pyrimidin-2-amine

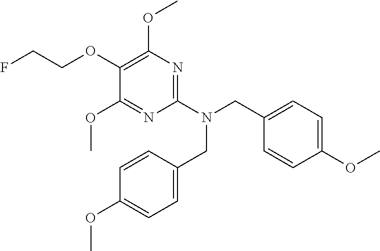 [SMILES: COc1ccc(CN(Cc2ccc(OC)cc2)c2nc(OC)c(OCCF)c(OC)n2)cc1]

A suspension of 2-[bis[(4-methoxyphenyl)methyl]amino]-4,6-dimethoxy-pyrimidin-5-ol (2 g, 4.52 mmol, intermediate A23, step 3), potassium carbonate (1.89 g, 13.56 mmol) and 1-bromo-2-fluoroethane (1.76 g, 1.03 mL, 13.56 mmol) in N,N-dimethylformamide (45 mL) was heated to 80° C. and stirred for 2.5 hours. The reaction mixture was poured into brine and extracted twice with ethyl acetate. The organic layers were dried over sodium sulfate, filtered and concentrated in vacuo. The residue was purified by flash chromatography over silica gel using a gradient ethylacetate/heptane 0-30% to provide the title compound as a white solid (1.79 g, 85% yield). MS (ESI) m/z=458.3 [M+H]⁺

### Step 2:5-(2-fluoroethoxy)-4,6-dimethoxy-pyrimidin-2-amine

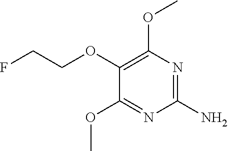

To a stirred solution of 5-(2-fluoroethoxy)-4,6-dimethoxy-N,N-bis[(4-methoxyphenyl)methyl]pyrimidin-2-amine (1.79 g, 3.83 mmol) in dichloromethane (4 mL) was added trifluoroacetic acid (26.23 g, 17.62 mL, 230.06 mmol). The reaction mixture was stirred at 50° C. for 3 hours, at room temperature for 15 hours and concentrated in vacuo. The residue was poured into sat. NaHCO₃ and extracted twice with ethyl acetate. The organic layers were dried over sodium sulfate, filtered and concentrated in vacuo. The residue was purified by flash chromatography over silica gel using a gradient ethylacetate/heptane 0-60% to provide the title compound as an off-white solid (885 mg, 100% yield). MS (ESI) m/z=218.1 [M+H]⁺

Intermediates B

| | | | | MS (ESI): |
|---|---|---|---|---|
| Interm. | | | | m/z, |
| number | structure | method | name | [M + H]⁺ |
| B1 | 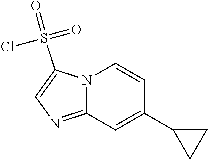 [SMILES: O=S(=O)(Cl)c1cnc2cc(C3CC3)ccn12] | Method B | 7-cyclopropylimidazo[1,2- a]pyridine-3-sulfonyl chloride | 257.1 |
| B2 | 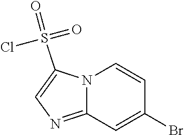 [SMILES: O=S(=O)(Cl)c1cnc2cc(Br)ccn12] | Method A | 7-bromoimidazo[1,2- a]pyridine-3-sulfonyl chloride | 297.0 |
| B3 | 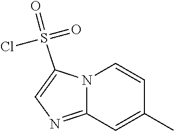 | Method A | 7-methylimidazo[1,2- a]pyridine-3-sulfonyl chloride | 231.1 |
| B4 | 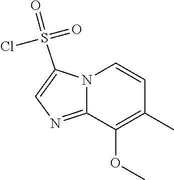 [SMILES: COc1c(C)ccn2c(S(=O)(=O)Cl)cnc12] | Method B | 8-methoxy-7-methyl- imidazo[1,2-a]pyridine-3- sulfonyl chloride | 261.1 |
| B5 | 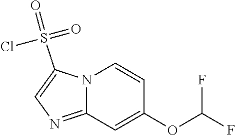 [SMILES: O=S(=O)(Cl)c1cnc2cc(OC(F)F)ccn12] | Method B | 7- (difluoromethoxy)imidazo[1,2- a]pyridine-3-sulfonyl chloride | 282.9 |
| B6 | 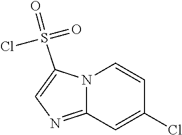 [SMILES: O=S(=O)(Cl)c1cnc2cc(Cl)ccn12] | Method C | 7-chloroimidazo[1,2- a]pyridine-3-sulfonyl chloride | 251.1 |
| B7 | 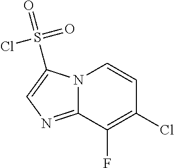 [SMILES: O=S(=O)(Cl)c1cnc2c(F)c(Cl)ccn12] | Method A | 7-chloro-8-fluoro-imidazo[1,2- a]pyridine-3-sulfonyl chloride | 269.0 |
| B8 | 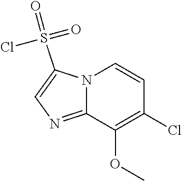 [SMILES: COc1c(Cl)ccn2c(S(=O)(=O)Cl)cnc12] | Method B | 7-chloro-8-methoxy- imidazo[1,2-a]pyridine-3- sulfonyl chloride | 281.0 |
| B9 | 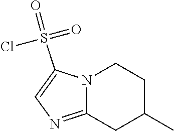 | Method A | 7-methyl-5,6,7,8- tetrahydroimidazo[1,2- a]pyridine-3-sulfonyl chloride | 235.0 |
| B10 | 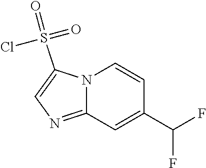 [SMILES: O=S(=O)(Cl)c1cnc2cc(C(F)F)ccn12] | Method B | 7-(difluoromethyl)imidazo[1,2- a]pyridine-3-sulfonyl chloride | 266.9 |
| B11 | 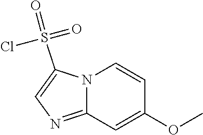 | Method B | 7-methoxyimidazo[1,2- a]pyridine-3-sulfonyl chloride | 247.1 |
| B12 | 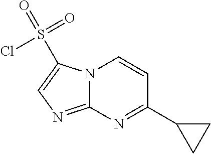 [SMILES: O=S(=O)(Cl)c1cnc2nc(C3CC3)ccn12] | Method B | 7-cyclopropylimidazo[1,2- a]pyrimidine-3-sulfonyl chloride | 258.1 |
| B13 | 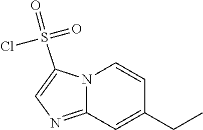 | Method B | 7-ethylimidazo[1,2-a]pyridine- 3-sulfonyl chloride | 245.0 |
| B14 | 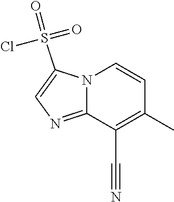 [SMILES: Cc1ccn2c(S(=O)(=O)Cl)cnc2c1C#N] | Method C | 8-cyano-7-methyl-imidazo[1,2- a]pyridine-3-sulfonyl chloride | 256.1 |
| B15 | 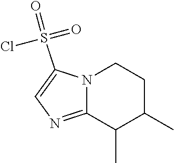 | Method A | 7,8-dimethyl-5,6,7,8- tetrahydroimidazo[1,2- a]pyridine-3-sulfonyl chloride | 249.1 |
| B16 | 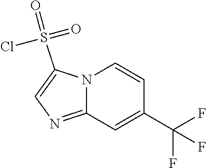 [SMILES: O=S(=O)(Cl)c1cnc2cc(C(F)(F)F)ccn12] | Method C | 7- (trifluoromethyl)imidazo[1,2- a]pyridine-3-sulfonyl chloride | 285.1 |
| B17 | 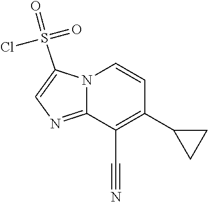 [SMILES: N#Cc1c(C2CC2)ccn2c(S(=O)(=O)Cl)cnc12] | Method C | 8-cyano-7-cyclopropyl- imidazo[1,2-a]pyridine-3- sulfonyl chloride | 282.1 |
| B18 | 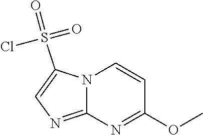 | Method B | 7-methoxyimidazo[1,2- a]pyrimidine-3-sulfonyl chloride | 248.0 |
| B19 | 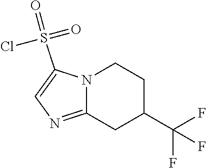 [SMILES: O=S(=O)(Cl)c1cnc2n1CCC(C(F)(F)F)C2] | Method A | 7-(trifluoromethyl)-5,6,7,8- tetrahydroimidazo[1,2- a]pyridine-3-sulfonyl chloride | 289.0 |
| B20 | 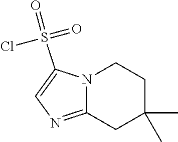 | Method A | 7,7-dimethyl-6,8-dihydro-5H- imidazo[1,2-a]pyridine-3- sulfonyl chloride | 249.1 |
| B21 | 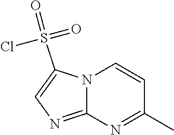 | Method B | 7-methylimidazo[1,2- a]pyrimidine-3-sulfonyl chloride | 232.1 |
| B22 | 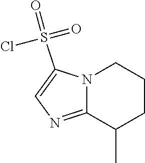 | Method A | 8-methyl-5,6,7,8- tetrahydroimidazo[1,2- a]pyridine-3-sulfonyl chloride | 235.1 |
| B23 | 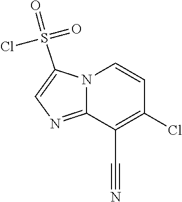 [SMILES: N#Cc1c(Cl)ccn2c(S(=O)(=O)Cl)cnc12] | Method C | 7-chloro-8-cyano-imidazo[1,2- a]pyridine-3-sulfonyl chloride | 276.0 |
| B24 | 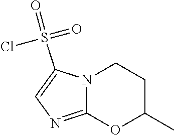 | Method A | 7-methyl-6,7-dihydro-5H- imidazo[2,1-b][1,3]oxazine-3- sulfonyl chloride | 237.0 |
| B25 | 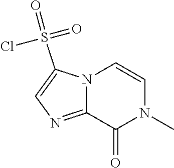 | Method C | 7-methyl-8-oxo-imidazo[1,2- a]pyrazine-3-sulfonyl chloride | 248.0 |
| B26 | 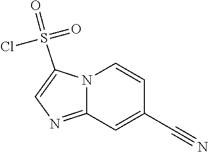 [SMILES: N#Cc1ccn2c(S(=O)(=O)Cl)cnc2c1] | Method C | 7-cyanoimidazo[1,2- a]pyridine-3-sulfonyl chloride | 242.1 |
| B27 | 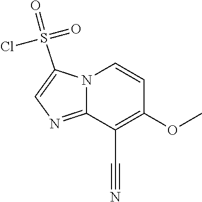 [SMILES: COc1ccn2c(S(=O)(=O)Cl)cnc2c1C#N] | Method C | 8-cyano-7-methoxy- imidazo[1,2-a]pyridine-3- sulfonyl chloride | 272.1 |
| B28 | 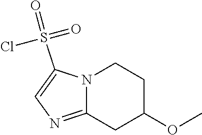 | Method C | 7-methoxy-5,6,7,8- tetrahydroimidazo[1,2- a]pyridine-3-sulfonyl chloride | 251.1 |
| B29 | 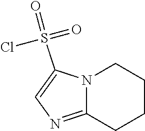 [SMILES: O=S(=O)(Cl)c1cnc2n1CCCC2] | Method A | 5,6,7,8-tetrahydroimidazo[1,2- a]pyridine-3-sulfonyl chloride | 220.7 |
| B30 | 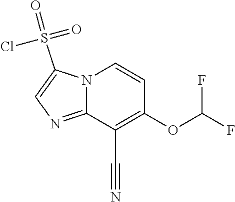 [SMILES: N#Cc1c(OC(F)F)ccn2c(S(=O)(=O)Cl)cnc12] | Method C | 8-cyano-7- (difluoromethoxy)imidazo[1,2- a]pyridine-3-sulfonyl chloride | 332.1 |
| B31 | 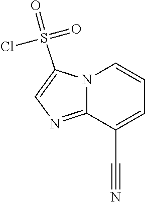 [SMILES: N#Cc1cccn2c(S(=O)(=O)Cl)cnc12] | Method C | 8-cyanoimidazo[1,2- a]pyridine-3-sulfonyl chloride | 242.1 |
| B32 | 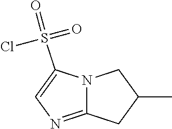 | Method A | 6-methyl-6,7-dihydro-5H- pyrrolo[1,2-a]imidazole-3- sulfonyl chloride | 221.0 |
| B33 | 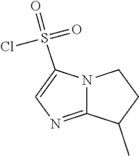 | Method A | 7-methyl-6,7-dihydro-5H- pyrrolo[1,2-a]imidazole-3- sulfonyl chloride | 221.0 |
| B34 | 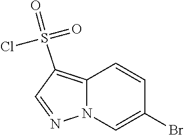 [SMILES: O=S(=O)(Cl)c1cnn2cc(Br)ccc12] | Method B | 6-bromopyrazolo[1,5- a]pyridine-3-sulfonyl chloride | 296.9 |
| B35 | 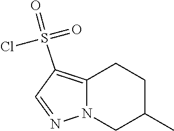 | Method A | 6-methyl-4,5,6,7- tetrahydropyrazolo[1,5- a]pyridine-3-sulfonyl chloride | 235.1 |
| B36 | 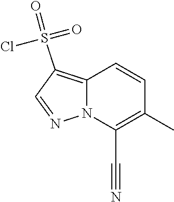 [SMILES: Cc1ccc2c(S(=O)(=O)Cl)cnn2c1C#N] | Method C | 7-cyano-6-methyl- pyrazolo[1,5-a]pyridine-3- sulfonyl chloride | 256.1 |
| B38 | 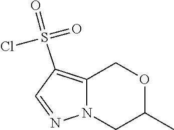 | Method A | 6-methyl-6,7-dihydro-4H- pyrazolo[5,1-c][1,4]oxazine-3- sulfonyl chloride | 219.0 (mass of acid) |
| B40 | 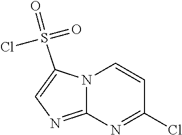 [SMILES: O=S(=O)(Cl)c1cnc2nc(Cl)ccn12] | Method A | 7-chloroimidazo [1,2- a]pyrimidine-3-sulfonyl chloride | 252.0 |

### Method A

A solution of imidazolo or pyrazolo derivative II or VIII or XIII or XVIII or XXIII (1 eq) in chlorosulfonic acid (12 eq) was stirred at 110° C. until LCMS indicated full conversion. The reaction mixture was allowed to cool to room temperature, poured into ice/ethyl acetate and extracted with ethyl acetate. The combined organic layers were washed twice with brine, dried over anhydrous sodium sulfate, filtered and concentrated in vacuo to give the corresponding sulfonyl chloride B. If sulfonyl chloride B is stable on silica gel, the crude material was purified by flash chromatography on silica gel using a gradient ethyl acetate/heptane.

### Method B

To a solution of imidazolo or pyrazolo derivative II or VIII or XIII or XVIII or XXIII (1 eq) in 1,2-dichloroethane was added trimethylsilyl chlorosulfonate (3 eq). The mixture was stirred at 90° C. until full conversion to sulfonic acid. The reaction mixture was cooled to room temperature, triethylamine (2 eq) and phosphorus oxychloride (3 eq) were added. The reaction mixture was stirred at 100° C. until LCMS showed full conversion. The reaction mixture was cooled to room temperature and poured into saturated NaHCO₃/ice solution and extracted with ethyl acetate. The combined organic layers were washed twice with brine, dried over anhydrous sodium sulfate, filtered and concentrated in vacuo to give the corresponding sulfonyl chloride B. If sulfonyl chloride B is stable on silica gel, the crude material was purified by flash chromatography on silica gel using a gradient ethyl acetate/heptane.

### Method C

Step 1: To a solution of imidazolo or pyrazolo derivative III or IX or XIV or XIX or XXIV (1 eq) in 1,4-dioxane (5 mL) under nitrogen at room temperature were added benzyl mercaptan (1.1 eq), N-ethyldiisopropylamine (2 eq), 9,9-dimethyl-4,5-bis(diphenylphosphino) xanthene (0.050 eq) and tris(dibenzylideneacetone) dipalladium (0.030 eq). The reaction mixture was stirred at 90° C. until full conversion before it was cooled to room temperature and poured into water. Ethyl acetate was added and the mixture was filtered. Both layers were separated and the aqueous phase was extracted twice with ethyl acetate. The combined organic layers were washed twice with brine, dried over anhydrous sodium sulfate, filtered and concentrated in vacuo. The crude material was purified by flash chromatography on silica gel using a gradient ethyl acetate/heptane to afford corresponding benzyl thioether.

Step 2: To a solution of benzyl thioether derivative (1 eq) in acetic acid/water (10:1) under nitrogen at room temperature was added n-chlorosuccinimide (3 eq). The reaction mixture was stirred at room temperature until full conversion. The reaction mixture was diluted with ice-water and ethyl acetate. Both layers were separated and the aqueous phase was extracted twice with ethyl acetate. The combined organic layers were washed twice with brine, dried over anhydrous sodium sulfate, filtered and concentrated in vacuo. If sulfonyl chloride B is stable on silica gel, the crude material was purified by flash chromatography on silica gel using a gradient ethyl acetate/heptane.

### Synthesis of Precursors I or III for Sulfonyl Chloride Intermediates B

Precursor B1-I: 7-cyclopropylimidazo[1,2-a]pyridine

In a 25 ml round-bottomed flask were added 7-bromoimidazo[1,2-a]pyridine (1 g, 5.08 mmol), cyclopropylboronic acid (566.77 mg, 6.6 mmol), 1,4-dioxane (24 mL), PdCl2(dppf)-CH₂Cl₂ (828.95 mg, 1.02 mmol) and cesium carbonate (4.96 g, 15.23 mmol). The reaction mixture was stirred at 90° C. for 2 h before it was poured into water and extracted with ethyl acetate. The combined organic layers were washed with brine, dried over anhydrous sodium sulfate, filtered and concentrated in vacuo. The crude material was adsorbed on ISOLUTE HM-N and purified by flash chromatography on silica gel using a gradient ethyl acetate/heptane 0-100% to afford 7-cyclopropylimidazo[1,2-a]pyridine (613 mg, 65%) as light brown oil. MS (ESI): m/z=159.1 [M+H]⁺

Precursor B2-I: 7-bromoimidazo[1,2-a]pyridine

Commercially available CAS 808744-34-5

Precursor B3-I: 7-methylimidazo[1,2-a]pyridine

Commercially available CAS 874-39-5

Precursor B4-I: 8-methoxy-7-methyl-imidazo[1,2-a]pyridine

To (3-methoxy-4-methyl-2-pyridyl)amine (0.500 g, 3.62 mmol) in ethanol (15 mL) was added chloroacetaldehyde 50% in water (5.68 g, 4.6 mL, 36.19 mmol) at room temperature. The reaction mixture was stirred at reflux for 16 h before it was cooled to room temperature and concentrated. The residue was dissolved in saturated in NaHCO₃ and extracted with ethyl acetate. The combined organic layers were washed with brine, dried over anhydrous sodium sulfate, filtered and concentrated in vacuo. The crude material was purified by flash chromatography on silica gel using a gradient methanol/dichloromethane 0-10% to afford 8-methoxy-7-methyl-imidazo[1,2-a]pyridine (478 mg, 77%) as light brown oil. MS (ESI): m/z=163.0 [M+H]⁺

Precursor B5-I: 7-(difluoromethoxy)imidazo[1,2-a]pyridine

To a solution of 4-(difluoromethoxy)pyridin-2-amine (20.0 mg, 0.120 mmol) in ethanol (1 mL) was added chloroacetaldehyde (51.48 mg, 0.260 mmol). The mixture was stirred at 90° C. for 16 h before it was concentrated under reduced pressure. The mixture was triturated with 5 mL acetonitrile and to give the 7-(difluoromethoxy)imidazo[1,2-a]pyridine (30 mg, 100%, 80% purity) as a yellow solid. 1H NMR (400 MHz, CHLOROFORM-d) 8=8.67 (br d, J=7.0 Hz, 1H), 8.00 (s, 1H), 7.94 (s, 1H), 7.79 (s, 1H), 7.19 (br d, J=5.4 Hz, 1H), 7.11-6.66 (m, 1H)

Precursor B6-III: 3-bromo-7-chloro-imidazo[1,2-a]pyridine Commercially available CAS 342613-67-6
Precursor B7-I: 7-chloro-8-fluoro-imidazo[1,2-a]pyridine Commercially available CAS 628691-87-2
Precursor B8-I: 7-chloro-8-methoxy-imidazo[1,2-a]pyridine

To a mixture of (4-chloro-3-methoxy-2-pyridyl)amine (200 mg, 1.26 mmol) in ethanol (1 mL) was added 2-chloroacetaldehyde (296.99 mg, 239.51 μL, 1.89 mmol). The reaction mixture was heated in a sand bath to 150° C. for 90 min before it was cooled to room temperature and concentrated in vacuo. The residue was treated with diethyl ether and filtered to afford 7-chloro-8-methoxy-imidazo[1,2-a]pyridine (247.5 mg, 104%) as light brown solid. MS (ESI): m/z=183.0 [M+H]⁺

Precursor B9-I: 7-methyl-5,6,7,8-tetrahydroimidazo[1,2-a]pyridine

To a solution of 7-methylimidazo[1,2-a]pyridine (1 g, 7.57 mmol) in 1-butanol (20.0 mL) was added Raney Ni (1.4 g). The reaction mixture was purged with hydrogen gas several times before it was stirred under hydrogen atmosphere (50 psi) at 65° C. until LCMS showed full conversion. The reaction mixture was filtered and concentrated under reduced pressure to give the 7-methyl-5,6,7,8-tetrahydroimidazo[1,2-a]pyridine (614 mg, 58%) as a light yellow oil. MS (ESI): m/z=137.2 [M+H]⁺

Precursor B10-I: 7-(difluoromethyl)imidazo[1,2-a]pyridine

To a solution of imidazo[1,2-a]pyridine-7-carboxaldehyde (500 mg, 3.42 mmol) in dichloromethane (20 mL) was added diethylaminosulfur trifluoride (1.93 g, 11.97 mmol) at −78° C. The mixture was stirred at 30° C. After 12 h LCMS showed ~80% of starting material remained. Diethylaminosulfur trifluoride (3.58 g, 22.24 mmol) was added to the reaction mixture and it was stirred at 30° C. for another 12 h before it was quenched by saturated NaHCO₃ solution and extracted with dichloromethane/MeOH (10:1 V/V). The combined organic layers were washed with brine, dried over anhydrous sodium sulfate, filtered and concentrated in vacuo. The crude material was purified by flash chromatography on silica gel using a gradient ethyl acetate/petroleum ether 0-50% to yield 7-(difluoromethyl)imidazo[1,2-a]pyridine (180 mg, 31%) as a yellow oil. 1H NMR (400 MHz, CHLOROFORM-d) 8=8.23 (d, J=7.0 Hz, 1H), 7.82-7.60 (m, 3H), 6.96 (d, J=7.0 Hz, 1H), 6.86-6.47 (m, 1H)

Precursor B11-I: 7-methoxyimidazo[1,2-a]pyridine

Commercially available CAS 342613-71-2

Precursor B12-I: 7-cyclopropylimidazo[1,2-a]pyrimidine

To a solution of (4-cyclopropylpyrimidin-2-yl)amine (1 g, 7.4 mmol) in ethanol (28.26 mL) was added chloroacetaldehyde 50% in water (11.61 g, 9.4 mL, 73.98 mmol) at room temperature. The reaction mixture was stirred at reflux for 16 h before it was cooled to room temperature and concentrated in vacuo. The residue was dissolved in saturated in NaHCO₃ and extracted with ethyl acetate. The combined organic layers were washed with brine, dried over anhydrous sodium sulfate, filtered and concentrated in vacuo. The crude material was purified by flash chromatography on silica gel using a gradient dichloromethane/methanol 0-7.5% to afford 7-cyclopropylimidazo[1,2-a]pyrimidine (670 mg, 51%) as a light red solid. MS (EI): m/z=158.1 [M−H]⁺

Precursor B13-I: 7-ethylimidazo[1,2-a]pyridine

To a solution of (4-ethyl-2-pyridyl)amine (500 mg, 3.97 mmol) in ethanol (16 mL) was added 2-chloroacetaldehyde 50% in water (6.23 g, 5.03 mL, 39.7 mmol) and the resulting yellow solution was stirred at 80° C. for 5 h. The reaction mixture was cooled to room temperature and diluted with sat. NaHCO₃ solution, extracted twice with ethyl acetate and the combined organic layers were washed with brine, dried over anhydrous sodium sulfate, filtered and concentrated in vacuo. The crude material was purified by flash chromatography on silica gel using a gradient dichloromethane/methanol 0-5% to afford 7-ethylimidazo[1,2-a]pyridine (313 mg, 49%) as a yellow oil. MS (ESI): m/z=147.1 [M+H]⁺

Precursor B14-III: 3-bromo-7-methyl-imidazo[1,2-a]pyridine-8-carbonitrile

To a solution of 7-methylimidazo[1,2-a]pyridine-8-carbonitrile (750 mg, 4.77 mmol) in acetonitrile (12 mL), N-bromosuccinimide (849.3 mg, 4.77 mmol) was added in portions and the resulting reaction mixture was stirred at 22° C. until full conversion. Sat. NaHCO₃ solution was added to the reaction mixture and it was extracted with ethyl acetate. The combined organic layers were washed with brine, dried over anhydrous sodium sulfate, filtered and concentrated in vacuo to afford 3-bromo-7-methyl-imidazo[1,2-a]pyridine-8-carbonitrile (990 mg, 88%) as orange solid. MS (ESI): m/z=236.0 [M+H]⁺

Precursor B15-I: 7,8-dimethyl-5,6,7,8-tetrahydroimidazo[1,2-a]pyridine

A reaction mixture of 7,8-dimethylimidazo[1,2-a]pyridine (550 mg, 4 mmol) and 4.5% palladium and 0.5% Rhodium on carbon (770 mg, 3.4 mol %) in ethanol (11 mL) was heated at 80° C. and 50 bar hydrogen atmosphere until full conversion. The reaction mixture was filtered and the crude material concentrated in vacuo to afford 7,8-dimethyl-5,6,7,8-tetrahydroimidazo[1,2-a]pyridine (469 mg, 63%) as a light yellow oil. MS (ESI): m/z=150.9 [M+H]⁺

Precursor B16-III: 3-bromo-7-(trifluoromethyl)imidazo[1,2-a]pyridine

To a stirred solution of 7-(trifluoromethyl)imidazo[1,2-a]pyridine (650 mg, 3.49 mmol) in acetic acid (8 mL) at room temperature was added bromine (558.09 mg, 178.93 μL, 3.49 mmol). A clear yellow solution was stirred at room temperature for 1 h before 4N NaOH were added at 0° C. until pH>10 was reached. The reaction mixture was extracted with dichloromethane/methanol 95:5. The combined organic layers were washed with brine, dried over anhydrous sodium sulfate, filtered and concentrated in vacuo to afford 3-bromo-7-(trifluoromethyl)imidazo[1,2-a]pyridine (810 mg, 70%) as light yellow solid. MS (ESI): m/z=265.1 [M+H]⁺

Precursor B17-III: 3-bromo-7-cyclopropyl-imidazo[1,2-a]pyridine-8-carbonitrile

Step 1: To a solution of 2-amino-4-bromo-nicotinonitrile (300 mg, 1.52 mmol), cyclopropylboronic acid (260.27 mg, 3.03 mmol) in toluene (6 mL) and water (0.3 mL) were added tricyclohexyl phosphine (84.97 mg, 0.303 mmol), palladium acetate (34.01 mg, 0.152 mmol) and tripotassium phosphate (1.13 g, 5.3 mmol). The reaction mixture was stirred at 110° C. for 15 h before it was concentrated in vacuo. The crude material was purified by flash chromatography on silica gel using a gradient heptane/ethyl acetate 0-100% to afford 2-amino-4-cyclopropyl-nicotinonitrile (124 mg, 51%) as a yellow solid. MS (ESI): m/z=160.1 [M+H]⁺

Step 2: To a yellow solution of 2-amino-4-cyclopropyl-nicotinonitrile (124 mg, 0.779 mmol) in ethanol (6.18 mL) was added chloroacetaldehyde (183.44 mg, 147.93 μL, 2.34 mmol). The reaction mixture was stirred for 4 h at 150° C. before was then concentrated in vacuo. The crude material was purified by flash chromatography on silica gel using a gradient methanol/ethyl acetate 0-50% to afford 7-cyclopropylimidazo[1,2-a]pyridine-8-carbonitrile (155 mg, 97%) as light brown solid. MS (ESI): m/z=184.1 [M+H]⁺

Step 3: To a suspension of 7-cyclopropylimidazo[1,2-a]pyridine-8-carbonitrile (155 mg, 0.761 mmol) in acetonitrile (2 mL) was added N-bromosuccinimide (135 mg, 0.761 mmol) in acetonitrile (1 mL) at 0° C. The resulting reaction mixture was stirred for 1 h at 0° C. before it was poured into sat. NaHCO₃ solution and extracted with ethyl acetate. The combined organic layers were washed with brine, dried over anhydrous sodium sulfate, filtered and concentrated in vacuo. The crude material was purified by reverse phase HPLC to afford 3-bromo-7-cyclopropyl-imidazo[1,2-a]pyridine-8-carbonitrile (153 mg, 63%) as light yellow solid. MS (ESI): m/z=262.1 [M+H]⁺

Precursor B18-I: 7-methoxyimidazo[1,2-a]pyrimidine

To a solution of (4-methoxypyrimidin-2-yl)amine (1 g, 7.83 mmol) in ethanol (29.4 mL) was added chloroacetaldehyde 50% in water (12.3 g, 9.95 mL, 78.32 mmol) at room temperature and the reaction mixture was stirred at reflux for 16 h. The reaction mixture was cooled to room temperature and diluted with sat. NaHCO₃ solution, extracted twice with ethyl acetate and the combined organic layers were washed with brine, dried over anhydrous sodium sulfate, filtered and concentrated in vacuo. The crude material was purified by flash chromatography on silica gel using a gradient dichloromethane/methanol 0-7.5% to afford 7-methoxyimidazo[1,2-a]pyrimidine (742 mg, 63%) as white solid. MS (ESI): m/z=150.0 [M+H]⁺

Precursor B19-I: 7-(trifluoromethyl)-5,6,7,8-tetrahydroimidazo[1,2-a]pyridine

A reaction mixture of 7-(trifluoromethyl)imidazo[1,2-a]pyridine (75 mg, 0.4 mmol) and 5% platinum on carbon (7.5 mg) in ethanol (2 mL) was heated at 80° C. and 50 bar hydrogen atmosphere until full conversion. The reaction mixture was filtered and the crude material concentrated in vacuo to afford 7-(trifluoromethyl)-5,6,7,8-tetrahydroimidazo[1,2-a]pyridine (72 mg, 73%) as a colorless solid. MS (EI+): m/z=190.1 [M]⁺

Precursor B20-I: 7,7-dimethyl-6,8-dihydro-5H-imidazo[1,2-a]pyridine

Step 1: A solution of 4,4-dimethyl-2-piperidone (1 g, 7.86 mmol, 1 eq) and Lawesson's reagent (1.74 g, 4.31 mmol) in tetrahydrofuran (13.35 mL) was stirred at 70° C. for 2 h. The reaction mixture was poured into water and extracted with ethyl acetate. The combined organic layers were washed with water, brine, dried over sodium sulfate, filtered and concentrated to dryness. The crude compound was purified by flash chromatography on silica gel using a gradient dichloromethane/methanol 0-30% to afford 4,4-dimethylpiperidine-2-thione (700 mg, 59%) as white solid. MS (ESI): m/z=144.1 [M+H]⁺

Step 2:4,4-dimethylpiperidine-2-thione (255 mg, 1.78 mmol) was dissolved in 1-butanol (8.9 mL) followed by the addition of 2,2-diethoxyethylamine (355 mg, 388.25 μL, 2.67 mmol) and p-toluenesulfonic acid monohydrate (507 mg, 2.67 mmol). The mixture was stirred at 130° C. for 16 h. After full conversion the solvent was removed and mixture was poured into dichloromethane and sat. NaHCO₃ solution. Layers were separated and the aqueous layer was extracted with DCM. The combined organic layers were washed with water, brine, dried over sodium sulfate, filtered and concentrated to dryness. The crude was purified by flash chromatography on silica gel using a gradient 0-30% methanol in dichloromethane (stain with KMnO4) to afford 7,7-dimethyl-6,8-dihydro-5H-imidazo[1,2-a]pyridine (67 mg, 24%) as light yellow oil. MS (ESI): m/z=151.1 [M+H]⁺

Precursor B21-I: 7-methylimidazo[1,2-a]pyrimidine

To a solution of (4-methylpyrimidin-2-yl)amine (3 g, 27.49 mmol) in ethanol (120 mL) was added chloroacetaldehyde, 50% in water (43.16 g, 34.92 mL, 274.9 mmol) at room temperature, and the reaction mixture was stirred at reflux for 16 h. The reaction mixture was cooled to room temperature and diluted with sat. NaHCO₃ solution, extracted twice with ethyl acetate and the combined organic layers were washed with brine, dried over anhydrous sodium sulfate, filtered and concentrated in vacuo. The crude material was purified by flash chromatography on silica gel using a gradient dichloromethane/methanol 0-10% to afford a regioisomeric mixture which was separated by SFC. 7-methylimidazo[1,2-a]pyrimidine (466 mg, 12%) was obtained as a light yellow solid. MS (ESI): m/z=134.0 [M+H]⁺

Precursor B22-I: 8-methyl-5,6,7,8-tetrahydroimidazo[1,2-a]pyridine

Step 1: A solution of 3-methyl-2-piperidone (1 g, 8.84 mmol) and Lawesson's reagent (1.96 g, 4.84 mmol) in tetrahydrofuran (15 mL) was stirred at 70° C. for 2 h. The reaction mixture was poured into water and extracted with ethyl acetate. The combined organic layers were washed with water, brine, dried over sodium sulfate, filtered and concentrated to dryness. The crude material was purified by flash chromatography on silica gel using a gradient dichloromethane/methanol 0-30% to afford 3-methylpiperidine-2-thione (532 mg, 44%) as white solid. MS (ESI): m/z=130.1 [M+H]⁺

Step 2:3-methylpiperidine-2-thione (308 mg, 2.38 mmol) was dissolved in toluene (10 mL) followed by the addition of 2,2-dimethoxyethylamine (250.6 mg, 259.69 μL, 2.38 mmol) and p-toluenesulfonic acid monohydrate (453.4 mg, 2.38 mmol). The reaction mixture was stirred at 130° C. for 72 h. After full conversion, the reaction mixture was poured on dichloromethane and aqueous sat. NaHCO₃ solution. Layers were separated and the aqueous layer was extracted twice with dichloromethane. Combined organic layers were washed with water and brine, dried over sodium sulfate, filtered and concentrated to dryness to afford 8-methyl-5,6,7,8-tetrahydroimidazo[1,2-a]pyridine (215 mg, 39%) as brown solid which was used without further purification. MS (ESI): m/z=137.1 [M+H]⁺

Precursor B23-III: 3-bromo-7-chloro-imidazo[1,2-a]pyridine-8-carbonitrile

Step 1: A mixture of 2,4-dibromonicotinonitrile (2 g, 7.64 mmol) and ammonia (0.4 M in THF) (62.96 g, 80 mL, 32 mmol) was stirred at 100° C. for 16 h. After full conversion, the reaction mixture was concentrated in vacuo. The crude material was purified by flash chromatography on silica gel using a gradient heptane/ethyl acetate 0-100% to afford 2-amino-4-bromo-nicotinonitrile (140.2 mg, 8%) as white solid. MS (GS): m/z=196.9 [M]⁺

Step 2: To a mixture of 2-amino-4-methoxy-nicotinonitrile (2.03 g, 13.61 mmol) in ethanol (5.1 mL) was added 2-chloroacetaldehyde (3.2 g, 2.59 mL, 20.41 mmol). The reaction mixture was subjected to microwave heating at 150° C. for 45 min. After cooling to room temperature, the solvent was removed under reduced pressure. The residue was treated with diethyl ether leading to solid precipitation. The solid was filtered off, washed with ethyl acetate and dried under reduced pressure to yield 7-hydroxyimidazo[1,2-a]pyridine-8-carbonitrile (802 mg, 33%) as brown powder: MS (ESI): m/z=160.0 [M+H]⁺

Step 3: A suspension of 7-hydroxyimidazo[1,2-a]pyridine-8-carbonitrile (200 mg, 1.13 mmol) in phosphoryl chloride (888.3 mg, 540 μL, 5.79 mmol) was heated to 140° C. for 2 h. The reaction mixture was concentrated in vacuo and the residue was treated with aqueous sat. NaHCO₃ and extracted twice with dichloromethane. The organic layers were dried over sodium sulfate, filtered and concentrated to dryness to afford 7-chloroimidazo[1,2-a]pyridine-8-carbonitrile (162 mg, 80%) as yellow solid. MS (ESI): m/z=178.1 [M+H]⁺

Step 4: A suspension of 7-chloroimidazo[1,2-a]pyridine-8-carbonitrile (142 mg, 0.800 mmol) in acetonitrile (7 mL) was cooled to 0° C. A solution of N-bromosuccinimide (143 mg, 0.800 mmol) in acetonitrile (1.5 mL) was added dropwise. The reaction mixture was stirred at 0° C. for 25 min before it was poured into aqueous sat. NaHCO₃ and extracted twice with ethyl acetate The organic layers were dried over sodium sulfate, filtered and concentrated to dryness. The crude material was purified by flash chromatography on silica gel using a gradient heptane/ethyl acetate 0-60% to afford 3-bromo-7-chloro-imidazo[1,2-a]pyridine-8-carbonitrile (169 mg, 73%) as light yellow solid. MS (ESI): m/z=258.0 [M+H]⁺

Precursor B24-I: 7-methyl-6,7-dihydro-5H-imidazo[2,1-b][1,3]oxazine

A mixture of 2-chloro-1H-imidazole (1.1 g, 10.73 mmol), potassium carbonate (2.97 g, 21.46 mmol) and 4-bromobutan-2-ol (1.64 g, 10.73 mmol) in acetonitrile (22 mL) was stirred at 60° C. overnight. The solid was filtered off, washed with acetonitrile and the filtrate was concentrated in vacuo. The residue was dissolved in tetrahydrofuran (22 mL), sodium hydride (429 mg, 10.73 mmol) was added at 0° C. and the resulting reaction mixture was stirred at 70° C. overnight. The reaction mixture was quenched with acetic acid (644.31 mg, 614.22 μL, 10.73 mmol), and concentrated in vacuo. The crude material was purified by flash on silica gel using a gradient heptane/ethyl acetate 0-100% to afford 7-methyl-6,7-dihydro-5H-imidazo[2,1-b][1,3]oxazine as a colorless semisolid. MS (EI+): m/z=138.0 [M]⁺

Precursor B25-III: 3-bromo-7-methyl-imidazo[1,2-a]pyrazin-8-one

A suspension of 3-bromo-7H-imidazo[1,2-a]pyrazin-8-one (350 mg, 1.64 mmol, CAS: 689297-67-4) in N,N-dimethylformamide (7 mL) was cooled to 8-10° C. and sodium hydride (78.5 mg, 1.96 mmol) was added portionwise. After 15 min stirring at 20° C. iodomethane (255.34 mg, 112.48 μL, 1.8 mmol) was added and the reaction mixture was stirred at room temperature. After 1 h the suspension was cooled to 0° C., methanol (2 ml) was added slowly and stirred for 10 min before concentrating in vacuo. The crude compound was purified by flash chromatography on silica gel using a gradient dichloromethane/methanol 0-15% to afford 3-bromo-7-methyl-imidazo[1,2-a]pyrazin-8-one (280 mg, 75%) as white solid. MS (ESI): m/z=230.1 [M+H]⁺

Precursor B26-III: 3-bromoimidazo[1,2-a]pyridine-7-carbonitrile

Commercially available CAS 1392210-94-4

Precursor B27-III: 3-bromo-7-methoxy-imidazo[1,2-a]pyridine-8-carbonitrile

Commercially available CAS 1072944-45-6

Precursor B28-III: 3-bromo-7-methoxy-5,6,7,8-tetrahydroimidazo[1,2-a]pyridine

Step 1: A reaction mixture of 7-methoxyimidazo[1,2-a]pyridine (1.9 g, 13 mmol) and 4.5% palladium and 0.5% Rhodium on carbon (1 g, 13 mol %) in ethanol (70 mL) was heated at 80° C. and 50 bar hydrogen atmosphere until full conversion. The reaction mixture was filtered and the crude material concentrated in vacuo to afford 7-methoxy-5,6,7,8-tetrahydroimidazo[1,2-a]pyridine (2 g, 82%) as a light yellow oil. MS (ESI): m/z=153.1 [M+H]⁺

Step 2: A mixture of 7-methoxy-5,6,7,8-tetrahydroimidazo[1,2-a]pyridine (380 mg, 2 mmol) and N-bromosuccinimide (355 mg, 2 mmol) in acetonitrile (5 mL) was stirred at room temperature. After 1 h the reaction mixture was poured into water and extracted with ethyl acetate. The combined organic layers were washed with brine, dried over sodium sulfate, filtered and concentrated in vacuo. The crude compound was purified by flash chromatography on silica gel using a gradient dichloromethane/methanol 0-10% to afford 3-bromo-7-methoxy-5,6,7,8-tetrahydroimidazo[1,2-a]pyridine (257 mg, 54%) as a colorless oil. MS (EI+): m/z=230.0 [M]⁺

Precursor B29-I: 5,6,7,8-tetrahydroimidazo[1,2-a]pyridine

Commercially available CAS 34167-66-3

Precursor B30-III: 3-bromo-7-(difluoromethoxy)imidazo[1,2-a]pyridine-8-carbonitrile

Step 1: A mixture of 2-amino-4-methoxy-nicotinonitrile (1 g, 6.57 mmol) and 2-chloroacetaldehyde solution,~50 wt. % in water (1.55 g, 1.25 mL, 9.85 mmol) in ethanol (2.5 mL) was heated to 150° C. and stirred for 5 h the reaction mixture was concentrated in vacuo. The residue was triturated with ethyl acetate and filtered through sintered glass. The solid was washed with diethyl ether and dried in vacuo to afford 7-hydroxyimidazo[1,2-a]pyridine-8-carbonitrile (1.23 g, 94%) as light brown solid. MS (ESI): m/z=160.1 [M+H]⁺

Step 2: A mixture of 7-hydroxyimidazo[1,2-a]pyridine-8-carbonitrile (400 mg, 2.51 mmol), cesium carbonate (1.24 g, 3.77 mmol), chlorodifluoroacetic acid sodium salt (997 mg, 6.28 mmol) and water (600 μL) in N,N-dimethylformamide (6 mL) was heated to 100° C. After 1 h the reaction mixture was poured into water and extracted twice with ethyl acetate. The organic layers were washed with brine, dried over sodium sulfate and concentrated in vacuo. The crude material was purified by flash on silica gel using a gradient heptane/ethyl acetate 0-100% to afford 7-(difluoromethoxy)imidazo[1,2-a]pyridine-8-carbonitrile (126 mg, 23%) as yellow solid. MS (ESI): m/z=210.0 [M+H]⁺

Step 3: A solution of 7-(difluoromethoxy)imidazo[1,2-a]pyridine-8-carbonitrile (36 mg, 0.172 mmol) in acetonitrile (1.5 mL) was cooled to 0° C. A solution of N-bromosuccinimide (31 mg, 0.172 mmol in acetonitrile (300 μL) was added dropwise. The reaction mixture was stirred at 0° C. for 35 min before it was poured into sat. NaHCO₃ and extracted twice with ethyl acetate. The organic layers were dried over sodium sulfate, filtered and concentrated in vacuo. The crude material was purified by flash on silica gel using a gradient heptane/ethyl acetate 0-100% to afford 3-bromo-7-(difluoromethoxy)imidazo[1,2-a]pyridine-8-carbonitrile (37 mg, 74%) as light yellow solid. MS (ESI): m/z=290.0 [M+H]⁺

Precursor B31-III: 3-bromoimidazo[1,2-a]pyridine-8-carbonitrile

Commercially available CAS 1383718-53-3

Precursor B32-I: 6-methyl-6,7-dihydro-5H-pyrrolo[1,2-a]imidazole

Step 1: A solution of 4-methyl-2-pyrrolidone (500 mg, 5.04 mmol) and Lawesson's reagent (1.12 g, 2.76 mmol) in tetrahydrofuran (10 mL) was stirred at 70° C. for 2 h before the reaction mixture was poured into water and extracted with ethyl acetate. The combined organic layers were washed with water, brine, dried over sodium sulfate, filtered and concentrated to dryness. The crude compound was purified by flash chromatography on silica gel using a gradient dichloromethane/methanol 0-10% to afford 4-methylpyrrolidine-2-thione (390 mg, 63%) as off-white solid. MS (ESI): m/z=116.0 [M+H]⁺

Step 2: A solution of 4-methylpyrrolidine-2-thione (390 mg, 3.22 mmol) and iodomethane (2.5 g, 1.1 mL, 17.59 mmol) in dichloromethane (12.14 mL) was stirred at 23° C. for 16 h. The reaction mixture diluted with sat. Na2CO3 solution and extracted with dichloromethane. The combined organic layers were washed with water, brine, dried over sodium sulfate and concentrated to dryness to afford 4-methyl-2-(methylthio)-1-pyrroline (342 mg, 82%) as light yellow gum. MS (ESI): m/z=130.0 [M+H]⁺

Step 3: A solution of 4-methyl-2-(methylthio)-1-pyrroline (342 mg, 2.65 mmol) and 2,2-dimethoxyethylamine (417 mg, 432.54 μL, 3.97 mmol) in ethanol (3.63 mL) was stirred at 90° C. for 24 h. All volatiles were removed in vacuo to afford 2,2-dimethoxyethyl-(4-methyl-1-pyrrolin-2-yl)amine (487 mg, 98%) as off-white gum. MS (ESI): m/z=187.2 [M+H]⁺

Step 4: A solution of 2,2-dimethoxyethyl-(4-methyl-1-pyrrolin-2-yl)amine (100 mg, 0.537 mmol) in HCl (846 mg, 705.44 μL, 8.59 mmol) and methanol (1 mL) with a few drops of water was stirred at 90° C. for 24 h. The solvent was evaporated and the crude compound was treated with sat. NaHCO₃sol, and extracted with dichloromethane. The combined organic layer were washed with water, brine, dried over sodium sulfate, filtered and concentrated to dryness to afford 6-methyl-6,7-dihydro-5H-pyrrol [1,2-a]imidazole (41 mg, 62%) as light brown gum. MS (ESI): m/z=123.1 [M+H]⁺

Precursor B33-I: 7-methyl-6,7-dihydro-5H-pyrrolo[1,2-a]imidazole

Step 1: A solution of 3-methyl-2-pyrrolidone (500 mg, 5.04 mmol) and Lawesson's reagent (1.12 g, 2.76 mmol) in tetrahydrofuran (10 mL) was stirred at 70° C. for 2 h. The reaction mixture was poured into water and extracted with ethyl acetate. The combined organic layers were washed with water, brine, dried over sodium sulfate, filtered and concentrated to dryness. The crude compound was purified by flash chromatography on silica gel using a gradient dichloromethane/methanol 0-10% to afford 3-methylpyrrolidine-2-thione (422 mg, 71%) as white solid. MS (ESI): m/z=116.0 [M+H]⁺

Step 2: A solution of 3-methylpyrrolidine-2-thione (422.4 mg, 3.59 mmol) and iodomethane (2.72 g, 1.2 mL, 19.19 mmol) in dichloromethane (7 mL) was stirred at 23° C. for 16 h. The reaction mixture was quenched with sat. Na2CO3 solution and extracted with dichloromethane. The combined organic layers were washed with water, brine, dried over sodium sulfate, filtered and concentrated to dryness to afford 3-methyl-2-(methylthio)-1-pyrroline (233 mg, 49%) as light yellow oil. MS (ESI): m/z=130.0 [M+H]⁺

Step 3: A solution of 3-methyl-2-(methylthio)-1-pyrroline (220 mg, 1.67 mmol) and 2,2-dimethoxyethylamine (210.37 mg, 218 μL, 2. mmol) in ethanol (2.2 mL) was stirred at 90° C. for 16 h. The reaction mixture was concentrated to dryness to afford 2,2-dimethoxyethyl-(3-methyl-1-pyrrolin-2-yl)amine (309 mg, 94%) as colorless oil. MS (ESI): m/z=187.1 [M+H]^{+Step} 4: A solution of 2,2-dimethoxyethyl-(3-methyl-1-pyrrolin-2-yl)amine (100 mg, 0.510 mmol) and p-toluenesulfonic acid monohydrate (19.4 mg, 0.102 mmol) in toluene (1 mL) was stirred at 130° C. for 4 h. The reaction mixture was quenched with sat. NaHCO₃sol, and extracted with dichloromethane. The combined organic layers were washed with brine, dried over sodium sulfate, filtered and concentrated to dryness to afford 7-methyl-6,7-dihydro-5H-pyrrol [1,2-a]imidazole (30 mg, 43%) as brown gum. MS (ESI): m/z=123.1 [M+H]⁺

Precursor B34-I: 6-bromopyrazolo[1,5-a]pyridine

Commercially available CAS 1264193-11-4

Precursor B35-I: 6-methyl-4,5,6,7-tetrahydropyrazolo[1,5-a]pyridine

Step 1: A solution of 6-bromopyrazolo[1,5-a]pyridine (400 mg, 2.03 mmol), 2,4,6-trimethyl-1,3,5,2,4,6-trioxatriborinane (509 mg, 566.32 μL, 4.06 mmol), potassium carbonate (308 mg, 2.23 mmol) in water (0.7 mL) and PdCl2(dppf)-CH2Cl2 adduct (82 mg, 0.102 mmol) in 1,4-dioxane (6.5 mL) was heated at 90° C. After 16 h the reaction was cooled to room temperature, ethyl acetate was added and the reaction mixture was filtered. All volatiles were removed under vacuo and the residue was purified by flash on silica gel using a gradient heptane/ethyl acetate 0-60% to afford 6-methylpyrazolo[1,5-a]pyridine (187 mg, 59%) as light yellow oil. MS (ESI): m/z=133.0 [M+H]⁺

Step 2:6-methylpyrazolo[1,5-a]pyridine (187 mg, 1.2 mmol) was dissolved in methanol (9 mL) and acetic acid (0.100 mL), platinum dioxide (27.31 mg, 0.120 mmol) was added and the reaction mixture was stirred under hydrogen at 35° C. After full conversion, the reaction mixture was filtered and washed with diethyl ether. The organic phase was diluted with diethyl ether and washed with water, dried over magnesium sulfate, filtered and concentrated in vacuo (30° C./200 mbar). The residue was diluted with diethyl ether and crystallized at 4° C. to obtain 6-methyl-4,5,6,7-tetrahydropyrazolo[1,5-a]pyridine (45 mg, contains acetic acid). The mother liquor was removed under vacuo to yield 6-methyl-4,5,6,7-tetrahydropyrazolo[1,5-a]pyridine (135 mg) as an oil containing ethyl acetate/acetic acid. MS (ESI): m/z=137.2 [M+H]⁺

Precursor B36-III: 3-bromo-6-methyl-pyrazolo[1,5-a]pyridine-7-carbonitrile

Step 1: T a solution of 6-bromo-3-methyl-picolinonitrile (2 g, 10.15 mmol) was dissolved in N,N-dimethylformamide (20 mL)trimethylsilylacetylene (1.2 g, 1.69 mL, 12.18 mmol), triphenylphosphine (532 mg, 2.03 mmol), bis(triphenylphosphine) palladium (II) chloride (712 mg, 1.02 mmol), triethylamine (3.08 g, 4.24 mL, 30.45 mmol) and copper (I) iodide (386 mg, 2.03 mmol) were added. The mixture was stirred at 80° C. After 16 h the solvent was removed under reduced pressure and the crude material was by flash on silica gel using a gradient heptane/ethyl acetate 0-100% to afford 3-methyl-6-(2-trimethylsilylethynyl) picolinonitrile (1.32 g, 59%) as a pink solid. MS (ESI): m/z=215.1 [M+H]⁺

Step 2:3-methyl-6-(2-trimethylsilylethynyl) picolinonitrile (1.32 g, 6.16 mmol) was dissolved in dichloromethane (16 mL) and the resulting solution was cold at 0° C. 2,4,6-trimethylbenzenesulfonic acid amino ester (2.39 g, 11.09 mmol) in dichloromethane (8 mL) was added dropwise and the mixture was stirred at 23° C. for 20 h. After completion, most of the solvent was removed under reduced pressure. Diethyl ether was added to the residue and a precipitate was formed. The solid was isolated by filtration to provide the desired product (1.24 g). The mother liquor was portioned between water and diethyl ether. Layers were separated and the ether phase was washed twice with water. The combined aqueous layers were dried on the lyophilizator to afford additional product. MS (ESI): m/z=230.2 [M+H]⁺

Step 3: A mixture of 1-amino-3-methyl-6-(2-trimethylsilylethynyl)pyridin-1-ium-2-carbonitrile. 1:1 2,4,6-trimethylbesylate (942 mg, 2.19 mmol) and K2CO3 (303 mg, 2.19 mmol) in N,N-dimethylformamide (15 mL) was stirred at 23° C. for 16 h. The solvent was removed under reduced pressure and the residue was portioned between ethyl acetate and water. Layers were separated, the aqueous layer was extracted twice with ethyl acetate. Combined organic layers were washed with brine, dried over sodium sulfate, filtered and concentrated under reduced pressure. The crude material was purified by flash on silica gel using a gradient heptane/ethyl acetate 0-60% to afford 6-methylpyrazolo[1,5-a]pyridine-7-carbonitrile (42 mg, 12%) as light yellow solid. MS (ESI): m/z=158.1 [M+H]⁺

Step 4: To a solution of 6-methylpyrazolo[1,5-a]pyridine-7-carbonitrile (18.3 mg, 0.114 mmol) in N,N-dimethylformamide (0.571 mL) was added N-bromosuccinimide (20.31 mg, 0.114 mmol). The resulting mixture was stirred at 25° C. for 2.5 h before it was portioned between ethyl acetate and water. Layers were separated and the organic layer was washed twice with water and brine. The combined organic layers were dried over sodium sulfate, filtered and concentrated under reduced pressure. The crude material was purified by flash on silica gel using a gradient heptane/ethyl acetate 0-100% to afford 3-bromo-6-methyl-pyrazolo[1,5-a]pyridine-7-carbonitrile (19 mg, 69%) as colorless solid. MS (ESI): m/z=236.0 [M+H]⁺

### EXAMPLES

### Example 1:7-bromo-N-[6-(2,2-difluoroethoxy)-5-fluoro-2-methoxy-3-pyridyl]imidazo[1,2-a]pyridine-3-sulfonamide

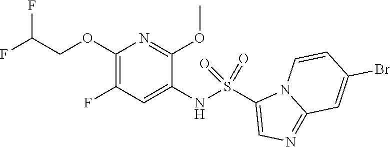 [SMILES: COc1nc(OCC(F)F)c(F)cc1NS(=O)(=O)c1cnc2cc(Br)ccn12]

To a mixture of 7-bromoimidazo[1,2-a]pyridine-3-sulfonyl chloride (Intermediate B2, 56 mg, 0.185 mmol) and [6-(2,2-difluoroethoxy)-5-fluoro-2-methoxy-3-pyridyl]amine (Intermediate A2, 41 mg, 0.185 mmol) was added pyridine (1 mL). The reaction mixture was stirred at room temperature. After full conversion the reaction mixture was concentrated in vacuo. The crude material was purified by flash on silica gel using a gradient heptane/ethyl acetate 0-60% to afford 7-bromo-N-[6-(2,2-difluoroethoxy)-5-fluoro-2-methoxy-3-pyridyl]imidazo[1,2-a]pyridine-3-sulfonamide (55 mg, 62%) as off-white solid. MS (ESI): m/z=483.1 [M+H]⁺

The following examples 2-88 were prepared in analogy to example 1 by coupling the indicated sulfonylchloride intermediates B and amine intermediates A.

| | | | | | MS (ESI): |
|---|---|---|---|---|---|
| | | | | | m/z, |
| Ex. | Structure | Name | Int. A | Int. B | [M + H]⁺ |
| 2 | 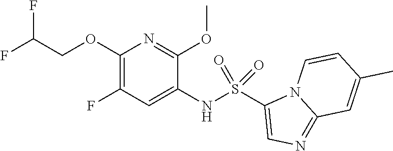 [SMILES: COc1nc(OCC(F)F)c(F)cc1NS(=O)(=O)c1cnc2cc(C)ccn12] | N-[6-(2,2-difluoroethoxy)- 5-fluoro-2-methoxy-3- pyridyl]-7-methyl- imidazo[1,2-a]pyridine-3- sulfonamide | A2 | B3 | 417.3 |
| 3 | 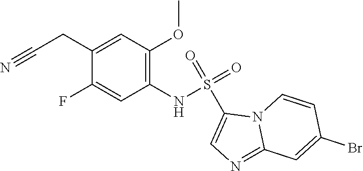 [SMILES: COc1cc(CC#N)c(F)cc1NS(=O)(=O)c1cnc2cc(Br)ccn12] | 7-bromo-N-[4- (cyanomethyl)-5-fluoro-2- methoxy- phenyl]imidazo[1,2- a]pyridine-3-sulfonamide | A1 | B2 | 441.2 |
| 4 | 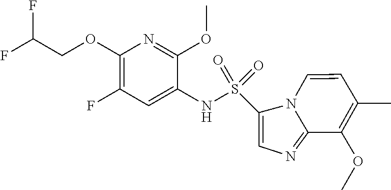 [SMILES: COc1nc(OCC(F)F)c(F)cc1NS(=O)(=O)c1cnc2c(OC)c(C)ccn12] | N-[6-(2,2-difluoroethoxy)- 5-fluoro-2-methoxy-3- pyridyl]-8-methoxy-7- methyl-imidazo[1,2- a]pyridine-3-sulfonamide | A2 | B4 | 447.2 |
| 5 | 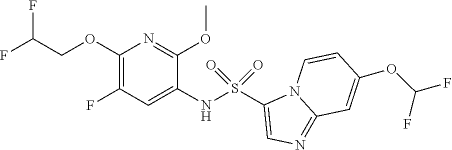 [SMILES: COc1nc(OCC(F)F)c(F)cc1NS(=O)(=O)c1cnc2cc(OC(F)F)ccn12] | N-[6-(2,2-difluoroethoxy)- 5-fluoro-2-methoxy-3- pyridyl]-7- (difluoromethoxy)imidazo [1,2-a]pyridine-3- sulfonamide | A2 | B5 | 469.0 |
| 6 | 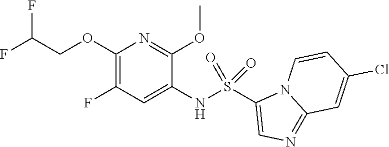 [SMILES: COc1nc(OCC(F)F)c(F)cc1NS(=O)(=O)c1cnc2cc(Cl)ccn12] | 7-chloro-N-[6-(2,2- difluoroethoxy)-5-fluoro-2- methoxy-3- pyridyl]imidazo[1,2- a]pyridine-3-sulfonamide | A2 | B6 | 437.2 |
| 7 | 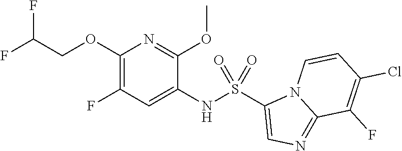 [SMILES: COc1nc(OCC(F)F)c(F)cc1NS(=O)(=O)c1cnc2c(F)c(Cl)ccn12] | 7-chloro-N-[6-(2,2- difluoroethoxy)-5-fluoro-2- methoxy-3-pyridyl]-8- fluoro-imidazo[1,2- a]pyridine-3-sulfonamide | A2 | B7 | 455.2 |
| 8 | 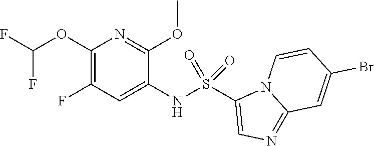 [SMILES: COc1nc(OC(F)F)c(F)cc1NS(=O)(=O)c1cnc2cc(Br)ccn12] | 7-bromo-N-[6- (difluoromethoxy)-5-fluoro- 2-methoxy-3- pyridyl]imidazo[1,2- a]pyridine-3-sulfonamide | A5 | B2 | 469.1 |
| 9 | 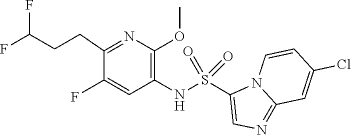 [SMILES: COc1nc(CCC(F)F)c(F)cc1NS(=O)(=O)c1cnc2cc(Cl)ccn12] | 7-chloro-N-[6-(3,3- difluoropropyl)-5-fluoro-2- methoxy-3- pyridyl]imidazo[1,2- a]pyridine-3-sulfonamide | A13 | B6 | 435.1 |
| 10 | 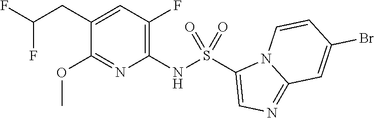 [SMILES: COc1nc(NS(=O)(=O)c2cnc3cc(Br)ccn23)c(F)cc1CC(F)F] | 7-bromo-N-[5-(2,2- difluoroethyl)-3-fluoro-6- methoxy-2- pyridyl]imidazo[1,2- a]pyridine-3-sulfonamide | A6 | B2 | 465.2 |
| 11 | 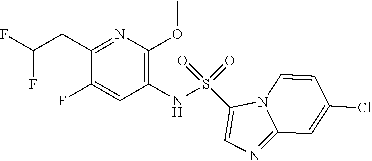 [SMILES: COc1nc(CC(F)F)c(F)cc1NS(=O)(=O)c1cnc2cc(Cl)ccn12] | 7-chloro-N-[6-(2,2- difluoroethyl)-5-fluoro-2- methoxy-3- pyridyl]imidazo[1,2- a]pyridine-3-sulfonamide | A7 | B6 | 421.1 |
| 12 | 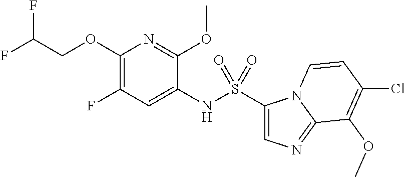 [SMILES: COc1nc(OCC(F)F)c(F)cc1NS(=O)(=O)c1cnc2c(OC)c(Cl)ccn12] | 7-chloro-N-[6-(2,2- difluoroethoxy)-5-fluoro-2- methoxy-3-pyridyl]-8- methoxy-imidazo[1,2- a]pyridine-3-sulfonamide | A2 | B8 | 467.1 |
| 13 | 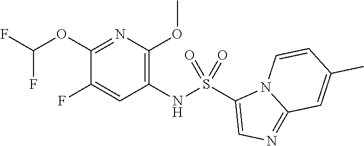 [SMILES: COc1nc(OC(F)F)c(F)cc1NS(=O)(=O)c1cnc2cc(C)ccn12] | N-[6-(difluoromethoxy)-5- fluoro-2-methoxy-3- pyridyl]-7-methyl- imidazo[1,2-a]pyridine-3- sulfonamide | A5 | B3 | 403.2 |
| 14 | 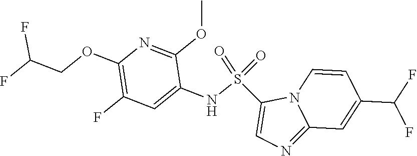 [SMILES: COc1nc(OCC(F)F)c(F)cc1NS(=O)(=O)c1cnc2cc(C(F)F)ccn12] | N-[6-(2,2-difluoroethoxy)- 5-fluoro-2-methoxy-3- pyridyl]-7- (difluoromethyl)imidazo[1,2- a]pyridine-3-sulfonamide | A2 | B10 | 453.1 |
| 15 | 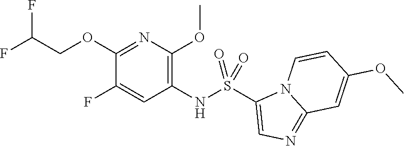 [SMILES: COc1ccn2c(S(=O)(=O)Nc3cc(F)c(OCC(F)F)nc3OC)cnc2c1] | N-[6-(2,2-difluoroethoxy)- 5-fluoro-2-methoxy-3- pyridyl]-7-methoxy- imidazo[1,2-a]pyridine-3- sulfonamide | A2 | B11 | 433.3 |
| 16 | 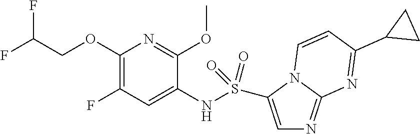 [SMILES: COc1nc(OCC(F)F)c(F)cc1NS(=O)(=O)c1cnc2nc(C3CC3)ccn12] | 7-cyclopropyl-N-[6-(2,2- difluoroethoxy)-5-fluoro-2- methoxy-3- pyridyl]imidazo[1,2- a]pyrimidine-3-sulfonamide | A2 | B12 | 444.2 |
| 17 | 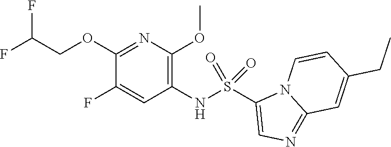 [SMILES: CCc1ccn2c(S(=O)(=O)Nc3cc(F)c(OCC(F)F)nc3OC)cnc2c1] | N-[6-(2,2-difluoroethoxy)- 5-fluoro-2-methoxy-3- pyridyl]-7-ethyl- imidazo[1,2-a]pyridine-3- sulfonamide | A2 | B13 | 431.2 |
| 18 | 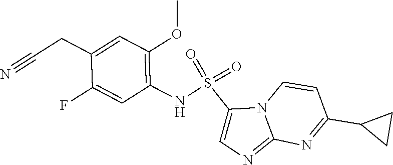 [SMILES: COc1cc(CC#N)c(F)cc1NS(=O)(=O)c1cnc2nc(C3CC3)ccn12] | N-[4-(cyanomethyl)-5- fluoro-2-methoxy-phenyl]- 7-cyclopropyl-imidazo[1,2- a]pyrimidine-3-sulfonamide | A1 | B12 | 402.3 |
| 19 | 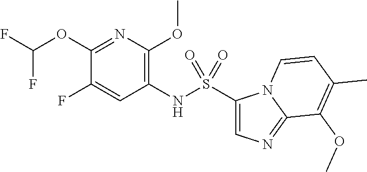 [SMILES: COc1nc(OC(F)F)c(F)cc1NS(=O)(=O)c1cnc2c(OC)c(C)ccn12] | N-[6-(difluoromethoxy)-5- fluoro-2-methoxy-3- pyridyl]-8-methoxy-7- methyl-imidazo[1,2- a]pyridine-3-sulfonamide | A5 | B4 | 433.2 |
| 20 | 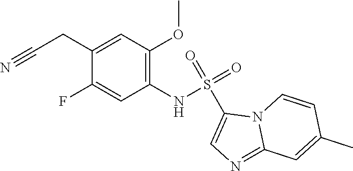 [SMILES: COc1cc(CC#N)c(F)cc1NS(=O)(=O)c1cnc2cc(C)ccn12] | N-[4-(cyanomethyl)-5- fluoro-2-methoxy-phenyl]- 7-methyl-imidazo[1,2- a]pyridine-3-sulfonamide | A1 | B3 | 375.2 |
| 21 | 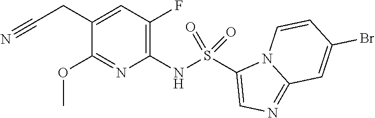 [SMILES: COc1nc(NS(=O)(=O)c2cnc3cc(Br)ccn23)c(F)cc1CC#N] | 7-bromo-N-[5- (cyanomethyl)-3-fluoro-6- methoxy-2- pyridyl]imidazo[1,2- a]pyridine-3-sulfonamide | A8 | B2 | 442.1 |
| 22 | 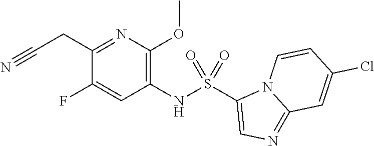 [SMILES: COc1nc(CC#N)c(F)cc1NS(=O)(=O)c1cnc2cc(Cl)ccn12] | 7-chloro-N-[6- (cyanomethyl)-5-fluoro-2- methoxy-3- pyridyl]imidazo[1,2- a]pyridine-3-sulfonamide | A3 | B6 | 396.0 |
| 23 | 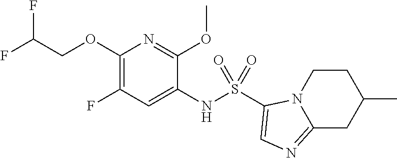 [SMILES: COc1nc(OCC(F)F)c(F)cc1NS(=O)(=O)c1cnc2n1CCC(C)C2] | N-[6-(2,2-difluoroethoxy)- 5-fluoro-2-methoxy-3- pyridyl]-7-methyl-5,6,7,8- tetrahydroimidazo[1,2- a]pyridine-3-sulfonamide | A2 | B9 | 420.9 |
| 24 | 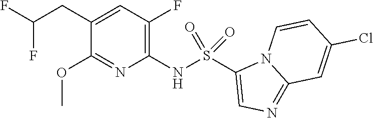 [SMILES: COc1nc(NS(=O)(=O)c2cnc3cc(Cl)ccn23)c(F)cc1CC(F)F] | 7-chloro-N-[5-(2,2- difluoroethyl)-3-fluoro-6- methoxy-2- pyridyl]imidazo[1,2- a]pyridine-3-sulfonamide | A6 | B6 | 421.1 |
| 25 | 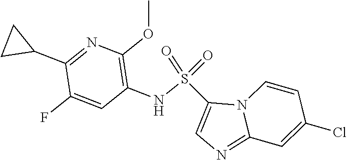 [SMILES: COc1nc(C2CC2)c(F)cc1NS(=O)(=O)c1cnc2cc(Cl)ccn12] | 7-chloro-N-(6-cyclopropyl- 5-fluoro-2-methoxy-3- pyridyl)imidazo[1,2- a]pyridine-3-sulfonamide | A9 | B6 | 397.1 |
| 26 | 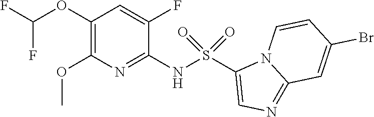 [SMILES: COc1nc(NS(=O)(=O)c2cnc3cc(Br)ccn23)c(F)cc1OC(F)F] | 7-bromo-N-[5- (difluoromethoxy)-3-fluoro- 6-methoxy-2- pyridyl]imidazo[1,2- a]pyridine-3-sulfonamide | A4 | B2 | 469.1 |
| 27 | 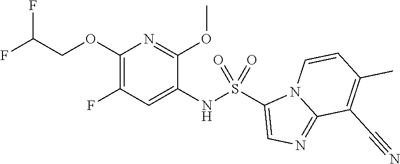 [SMILES: COc1nc(OCC(F)F)c(F)cc1NS(=O)(=O)c1cnc2c(C#N)c(C)ccn12] | 8-cyano-N-[6-(2,2- difluoroethoxy)-5-fluoro-2- methoxy-3-pyridyl]-7- methyl-imidazo[1,2- a]pyridine-3-sulfonamide | A2 | B14 | 442.2 |
| 28 | 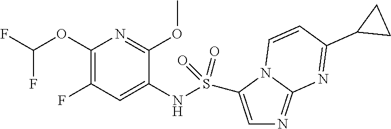 [SMILES: COc1nc(OC(F)F)c(F)cc1NS(=O)(=O)c1cnc2nc(C3CC3)ccn12] | 7-cyclopropyl-N-[6- (difluoromethoxy)-5-fluoro- 2-methoxy-3- pyridyllimidazo[1,2- a]pyrimidine-3-sulfonamide | A5 | B12 | 430.1 |
| 29 | 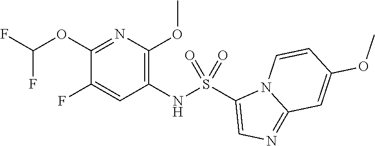 [SMILES: COc1ccn2c(S(=O)(=O)Nc3cc(F)c(OC(F)F)nc3OC)cnc2c1] | N-[6-(difluoromethoxy)-5- fluoro-2-methoxy-3- pyridyl]-7-methoxy- imidazo[1,2-a]pyridine-3- sulfonamide | A5 | B11 | 419.2 |
| 30 | 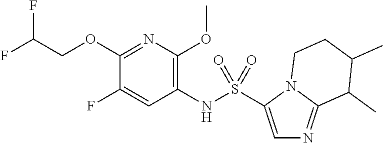 [SMILES: COc1nc(OCC(F)F)c(F)cc1NS(=O)(=O)c1cnc2n1CCC(C)C2C] | N-[6-(2,2-difluoroethoxy)- 5-fluoro-2-methoxy-3- pyridyl]-7,8-dimethyl- 5,6,7,8- tetrahydroimidazo[1,2- a]pyridine-3-sulfonamide | A2 | B15 | 435.2 |
| 31 | 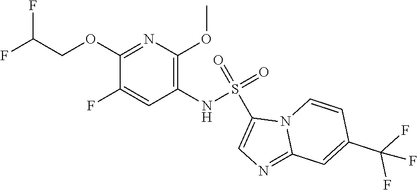 [SMILES: COc1nc(OCC(F)F)c(F)cc1NS(=O)(=O)c1cnc2cc(C(F)(F)F)ccn12] | N-[6-(2,2-difluoroethoxy)- 5-fluoro-2-methoxy-3- pyridyl]-7- (trifluoromethyl)imidazo [1,2-a]pyridine-3- sulfonamide | A2 | B16 | 471.2 |
| 32 | 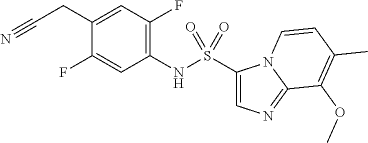 [SMILES: COc1c(C)ccn2c(S(=O)(=O)Nc3cc(F)c(CC#N)cc3F)cnc12] | N-[4-(cyanomethyl)-2,5- difluoro-phenyl]-8- methoxy-7-methyl- imidazo[1,2-a]pyridine-3- sulfonamide | A11 | B4 | 393.1 |
| 33 | 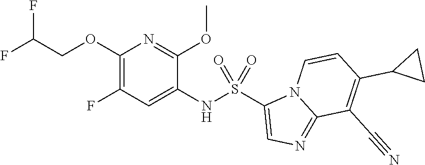 [SMILES: COc1nc(OCC(F)F)c(F)cc1NS(=O)(=O)c1cnc2c(C#N)c(C3CC3)ccn12] | 8-cyano-7-cyclopropyl-N- [6-(2,2-difluoroethoxy)-5- fluoro-2-methoxy-3- pyridyl]imidazo[1,2- a]pyridine-3-sulfonamide | A2 | B17 | 468.2 |
| 34 | 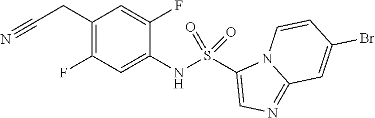 [SMILES: N#CCc1cc(F)c(NS(=O)(=O)c2cnc3cc(Br)ccn23)cc1F] | 7-bromo-N-[4- (cyanomethyl)-2,5-difluoro- phenyl]imidazo[1,2- a]pyridine-3-sulfonamide | A11 | B2 | 429.2 |
| 35 | 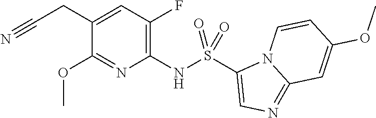 [SMILES: COc1ccn2c(S(=O)(=O)Nc3nc(OC)c(CC#N)cc3F)cnc2c1] | N-[5-(cyanomethyl)-3- fluoro-6-methoxy-2- pyridyl]-7-methoxy- imidazo[1,2-a]pyridine-3- sulfonamide | A8 | B11 | 392.2 |
| 36 | 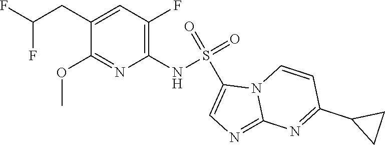 [SMILES: COc1nc(NS(=O)(=O)c2cnc3nc(C4CC4)ccn23)c(F)cc1CC(F)F] | 7-cyclopropyl-N-[5-(2,2- difluoroethyl)-3-fluoro-6- methoxy-2- pyridyl]imidazo[1,2- a]pyrimidine-3-sulfonamide | A6 | B12 | 428.2 |
| 37 | 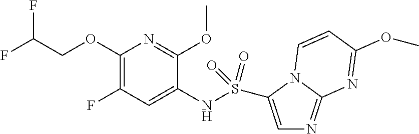 [SMILES: COc1ccn2c(S(=O)(=O)Nc3cc(F)c(OCC(F)F)nc3OC)cnc2n1] | N-[6-(2,2-difluoroethoxy)- 5-fluoro-2-methoxy-3- pyridyl]-7-methoxy- imidazo[1,2-a]pyrimidine-3- sulfonamide | A2 | B18 | 434.1 |
| 38 | 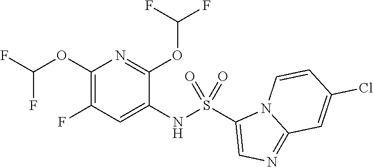 [SMILES: O=S(=O)(Nc1cc(F)c(OC(F)F)nc1OC(F)F)c1cnc2cc(Cl)ccn12] | N-[2,6- bis(difluoromethoxy)-5- fluoro-3-pyridyl]-7-chloro- imidazo[1,2-a]pyridine-3- sulfonamide | A14 | B6 | 459.1 |
| 39 | 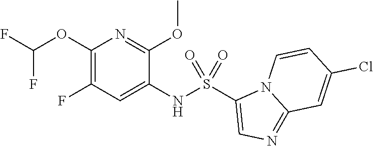 [SMILES: COc1nc(OC(F)F)c(F)cc1NS(=O)(=O)c1cnc2cc(Cl)ccn12] | 7-chloro-N-[6- (difluoromethoxy)-5-fluoro- 2-methoxy-3- pyridyl]imidazo[1,2- a]pyridine-3-sulfonamide | A5 | B6 | 423.1 |
| 40 | 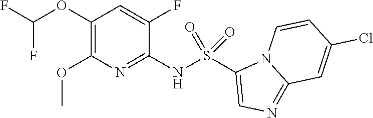 [SMILES: COc1nc(NS(=O)(=O)c2cnc3cc(Cl)ccn23)c(F)cc1OC(F)F] | 7-chloro-N-[5- (difluoromethoxy)-3-fluoro- 6-methoxy-2- pyridyl]imidazo[1,2- a]pyridine-3-sulfonamide | A4 | B6 | 423.1 |
| 41 | 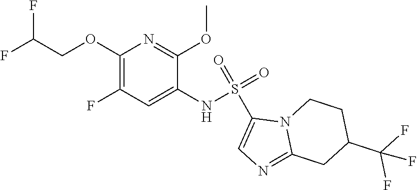 [SMILES: COc1nc(OCC(F)F)c(F)cc1NS(=O)(=O)c1cnc2n1CCC(C(F)(F)F)C2] | N-[6-(2,2-difluoroethoxy)- 5-fluoro-2-methoxy-3- pyridyl]-7- (trifluoromethyl)-5,6,7,8- tetrahydroimidazo[1,2- a]pyridine-3-sulfonamide | A2 | B20 | 475.2 |
| 42 | 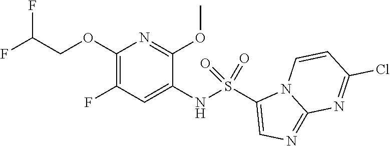 [SMILES: COc1nc(OCC(F)F)c(F)cc1NS(=O)(=O)c1cnc2nc(Cl)ccn12] | 7-chloro-N-[6-(2,2- difluoroethoxy)-5-fluoro-2- methoxy-3- pyridyl]imidazo[1,2- a]pyrimidine-3-sulfonamide | A2 | B40 | 438.2 |
| 43 | 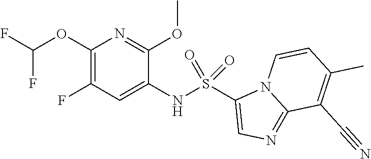 [SMILES: COc1nc(OC(F)F)c(F)cc1NS(=O)(=O)c1cnc2c(C#N)c(C)ccn12] | 8-cyano-N-[6- (difluoromethoxy)-5-fluoro- 2-methoxy-3-pyridyl]-7- methyl-imidazo[1,2- a]pyridine-3-sulfonamide | A5 | B14 | 428.2 |
| 44 | 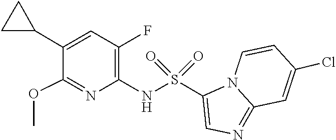 [SMILES: COc1nc(NS(=O)(=O)c2cnc3cc(Cl)ccn23)c(F)cc1C1CC1] | 7-chloro-N-(5-cyclopropyl- 3-fluoro-6-methoxy-2- pyridyl)imidazo[1,2- a]pyridine-3-sulfonamide | A15 | B6 | 397.1 |
| 45 | 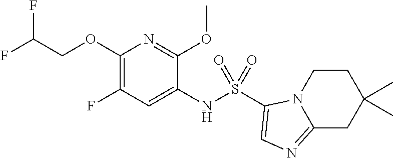 [SMILES: COc1nc(OCC(F)F)c(F)cc1NS(=O)(=O)c1cnc2n1CCC(C)(C)C2] | N-[6-(2,2-difluoroethoxy)- 5-fluoro-2-methoxy-3- pyridyl]-7,7-dimethyl-6,8- dihydro-5H-imidazo[1,2- a]pyridine-3-sulfonamide | A2 | B20 | 435.1 |
| 46 | 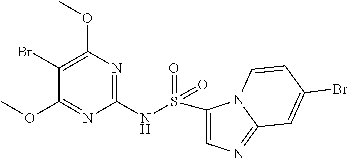 [SMILES: COc1nc(NS(=O)(=O)c2cnc3cc(Br)ccn23)nc(OC)c1Br] | 7-bromo-N-(5-bromo-4,6- dimethoxy-pyrimidin-2- yl)imidazo[1,2-a]pyridine- 3-sulfonamide | A16 | B2 | 493.9 |
| 47 | 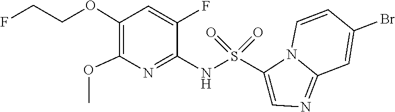 [SMILES: COc1nc(NS(=O)(=O)c2cnc3cc(Br)ccn23)c(F)cc1OCCF] | 7-bromo-N-[3-fluoro-5-(2- fluoroethoxy)-6-methoxy-2- pyridyl]imidazo[1,2- a]pyridine-3-sulfonamide | A10 | B2 | 465.1 |
| 48 | 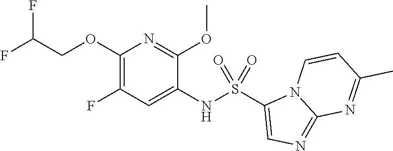 [SMILES: COc1nc(OCC(F)F)c(F)cc1NS(=O)(=O)c1cnc2nc(C)ccn12] | N-[6-(2,2-difluoroethoxy)- 5-fluoro-2-methoxy-3- pyridyl]-7-methyl- imidazo[1,2-a]pyrimidine-3- sulfonamide | A2 | B21 | 418.2 |
| 49 | 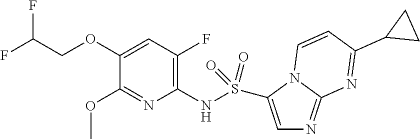 [SMILES: COc1nc(NS(=O)(=O)c2cnc3nc(C4CC4)ccn23)c(F)cc1OCC(F)F] | 7-cyclopropyl-N-[5-(2,2- difluoroethoxy)-3-fluoro-6- methoxy-2- pyridyl]imidazo[1,2- a]pyrimidine-3-sulfonamide | A12 | B12 | 444.1 |
| 50 | 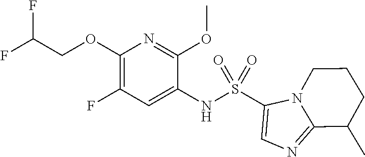 [SMILES: COc1nc(OCC(F)F)c(F)cc1NS(=O)(=O)c1cnc2n1CCCC2C] | N-[6-(2,2-difluoroethoxy)- 5-fluoro-2-methoxy-3- pyridyl]-8-methyl-5,6,7,8- tetrahydroimidazo[1,2- a]pyridine-3-sulfonamide | A2 | B22 | 421.1 |
| 51 | 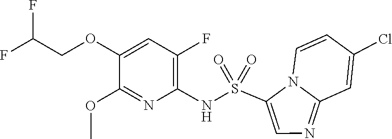 [SMILES: COc1nc(NS(=O)(=O)c2cnc3cc(Cl)ccn23)c(F)cc1OCC(F)F] | 7-chloro-N-[5-(2,2- difluoroethoxy)-3-fluoro-6- methoxy-2- pyridyl]imidazo[1,2- a]pyridine-3-sulfonamide | A12 | B6 | 437.1 |
| 52 | 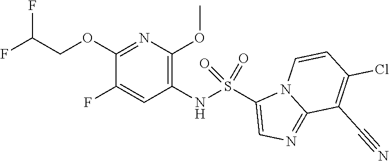 [SMILES: COc1nc(OCC(F)F)c(F)cc1NS(=O)(=O)c1cnc2c(C#N)c(Cl)ccn12] | 7-chloro-8-cyano-N-[6-(2,2- difluoroethoxy)-5-fluoro-2- methoxy-3- pyridyl]imidazo[1,2- a]pyridine-3-sulfonamide | A2 | B23 | 462.1 |
| 53 | 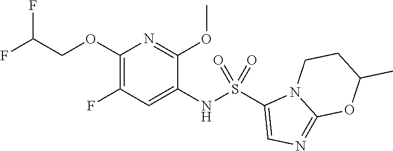 [SMILES: COc1nc(OCC(F)F)c(F)cc1NS(=O)(=O)c1cnc2n1CCC(C)O2] | N-[6-(2,2-difluoroethoxy)- 5-fluoro-2-methoxy-3- pyridyl]-7-methyl-6,7- dihydro-5H-imidazo[2,1- b][1,3]oxazine-3- sulfonamide | A2 | B24 | 423.2 |
| 54 | 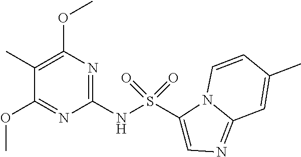 [SMILES: COc1nc(NS(=O)(=O)c2cnc3cc(C)ccn23)nc(OC)c1C] | N-(4,6-dimethoxy-5- methyl-pyrimidin-2-yl)-7- methyl-imidazo[1,2- a]pyridine-3-sulfonamide | A17 | B3 | 364.1 |
| 55 | 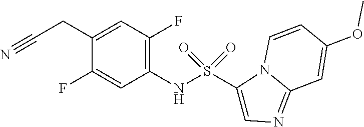 [SMILES: COc1ccn2c(S(=O)(=O)Nc3cc(F)c(CC#N)cc3F)cnc2c1] | N-[4-(cyanomethyl)-2,5- difluoro-phenyl]-7- methoxy-imidazo[1,2- a]pyridine-3-sulfonamide | A11 | B11 | 379.1 |
| 56 | 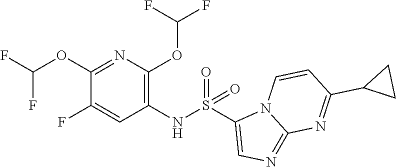 [SMILES: O=S(=O)(Nc1cc(F)c(OC(F)F)nc1OC(F)F)c1cnc2nc(C3CC3)ccn12] | N-[2,6- bis(difluoromethoxy)-5- fluoro-3-pyridyl]-7- cyclopropyl-imidazo[1,2- a]pyrimidine-3-sulfonamide | A14 | B12 | 466.1 |
| 57 | 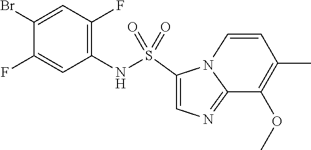 [SMILES: COc1c(C)ccn2c(S(=O)(=O)Nc3cc(F)c(Br)cc3F)cnc12] | N-(4-bromo-2,5-difluoro- phenyl)-8-methoxy-7- methyl-imidazo[1,2- a]pyridine-3-sulfonamide | A18 | B4 | 434.1 |
| 58 | 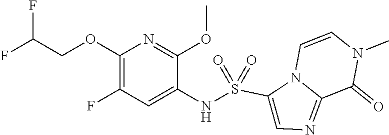 [SMILES: COc1nc(OCC(F)F)c(F)cc1NS(=O)(=O)c1cnc2c(=O)n(C)ccn12] | N-[6-(2,2-difluoroethoxy)- 5-fluoro-2-methoxy-3- pyridyl]-8-keto-7-methyl- imidazo[1,2-a]pyrazine-3- sulfonamide | A2 | B25 | 434.1 |
| 59 | 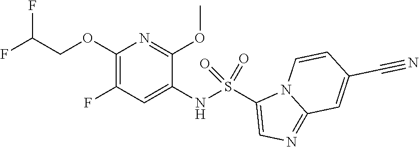 [SMILES: COc1nc(OCC(F)F)c(F)cc1NS(=O)(=O)c1cnc2cc(C#N)ccn12] | 7-cyano-N-[6-(2,2- difluoroethoxy)-5-fluoro-2- methoxy-3- pyridyl]imidazo[1,2- a]pyridine-3-sulfonamide | A2 | B26 | 428.3 |
| 60 | 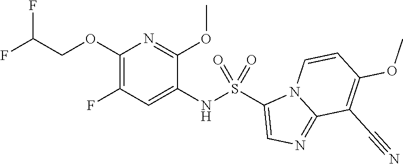 [SMILES: COc1ccn2c(S(=O)(=O)Nc3cc(F)c(OCC(F)F)nc3OC)cnc2c1C#N] | 8-cyano-N-[6-(2,2- difluoroethoxy)-5-fluoro-2- methoxy-3-pyridyl]-7- methoxy-imidazo[1,2- a]pyridine-3-sulfonamide | A2 | B27 | 458.1 |
| 61 | 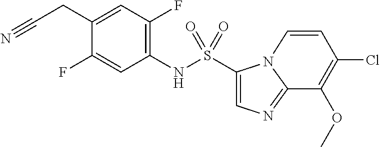 [SMILES: COc1c(Cl)ccn2c(S(=O)(=O)Nc3cc(F)c(CC#N)cc3F)cnc12] | 7-chloro-N-[4- (cyanomethyl)-2,5-difluoro- phenyl]-8-methoxy- imidazo[1,2-a]pyridine-3- sulfonamide | A11 | B8 | 413.1 |
| 62 | 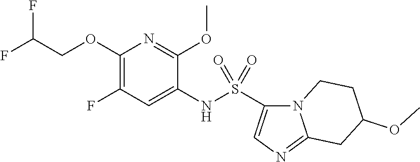 [SMILES: COc1nc(OCC(F)F)c(F)cc1NS(=O)(=O)c1cnc2n1CCC(OC)C2] | N-[6-(2,2-difluoroethoxy)- 5-fluoro-2-methoxy-3- pyridyl]-7-methoxy-5,6,7,8- tetrahydroimidazo[1,2- a]pyridine-3-sulfonamide | A2 | B28 | 437.2 |
| 63 | 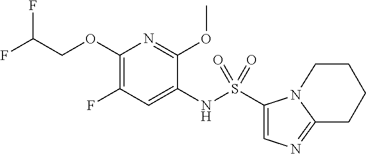 [SMILES: COc1nc(OCC(F)F)c(F)cc1NS(=O)(=O)c1cnc2n1CCCC2] | N-[6-(2,2-difluoroethoxy)- 5-fluoro-2-methoxy-3- pyridyl]-5,6,7,8- tetrahydroimidazo[1,2- a]pyridine-3-sulfonamide | A2 | B29 | 406.9 |
| 64 | 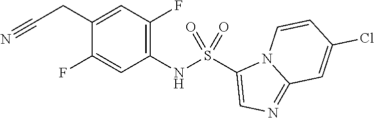 [SMILES: N#CCc1cc(F)c(NS(=O)(=O)c2cnc3cc(Cl)ccn23)cc1F] | 7-chloro-N-(4- (cyanomethyl)-2,5- difluorophenyl)imidazo[1,2- a]pyridine-3-sulfonamide | A11 | B6 | 383.1 |
| 65 | 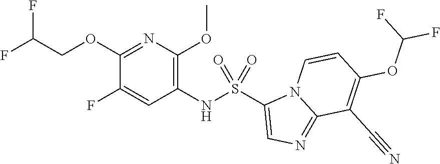 [SMILES: COc1nc(OCC(F)F)c(F)cc1NS(=O)(=O)c1cnc2c(C#N)c(OC(F)F)ccn12] | 8-cyano-N-[6-(2,2- difluoroethoxy)-5-fluoro-2- methoxy-3-pyridyl]-7- (difluoromethoxy)imidazo [1,2-a]pyridine-3- sulfonamide | A2 | B30 | 494.1 |
| 66 | 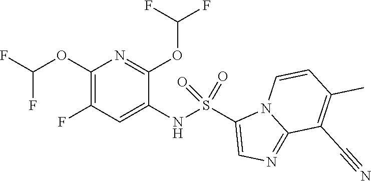 [SMILES: Cc1ccn2c(S(=O)(=O)Nc3cc(F)c(OC(F)F)nc3OC(F)F)cnc2c1C#N] | N-[2,6- bis(difluoromethoxy)-5- fluoro-3-pyridyl]-8-cyano- 7-methyl-imidazo[1,2- a]pyridine-3-sulfonamide | A14 | B14 | 464.1 |
| 67 | 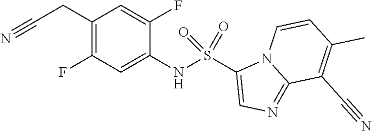 [SMILES: Cc1ccn2c(S(=O)(=O)Nc3cc(F)c(CC#N)cc3F)cnc2c1C#N] | 8-cyano-N-[4- (cyanomethyl)-2,5-difluoro- phenyl]-7-methyl- imidazo[1,2-a]pyridine-3- sulfonamide | A11 | B14 | 388.1 |
| 68 | 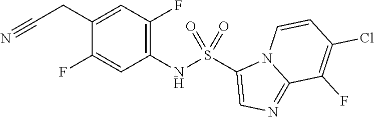 [SMILES: N#CCc1cc(F)c(NS(=O)(=O)c2cnc3c(F)c(Cl)ccn23)cc1F] | 7-chloro-N-[4- (cyanomethyl)-2,5-difluoro- phenyl]-8-fluoro- imidazo[1,2-a]pyridine-3- sulfonamide | A11 | B7 | 401.0 |
| 69 | 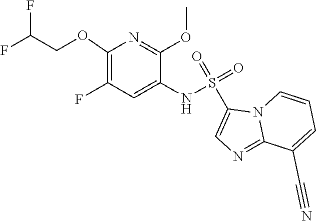 [SMILES: COc1nc(OCC(F)F)c(F)cc1NS(=O)(=O)c1cnc2c(C#N)cccn12] | 8-cyano-N-[6-(2,2- difluoroethoxy)-5-fluoro-2- methoxy-3- pyridyl]imidazo[1,2- a]pyridine-3-sulfonamide | A2 | B31 | 428.2 |
| 70 | 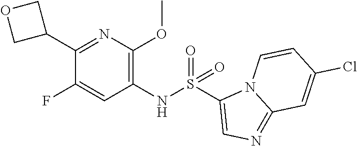 [SMILES: COc1nc(C2COC2)c(F)cc1NS(=O)(=O)c1cnc2cc(Cl)ccn12] | 7-chloro-N-[5-fluoro-2- methoxy-6-(oxetan-3-yl)-3- pyridyl]imidazo[1,2- a]pyridine-3-sulfonamide | A20 | B6 | 413.1 |
| 71 | 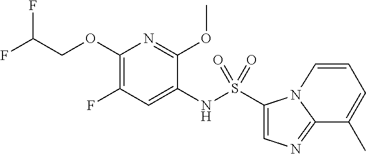 [SMILES: COc1nc(OCC(F)F)c(F)cc1NS(=O)(=O)c1cnc2c(C)cccn12] | N-[6-(2,2-difluoroethoxy)- 5-fluoro-2-methoxy-3- pyridyl]-8-methyl- imidazo[1,2-a]pyridine-3- sulfonamide | A2 | B32 | 417.1 |
| 72 | 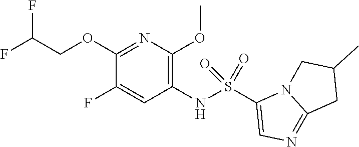 [SMILES: COc1nc(OCC(F)F)c(F)cc1NS(=O)(=O)c1cnc2n1CC(C)C2] | N-[6-(2,2-difluoroethoxy)- 5-fluoro-2-methoxy-3- pyridyl]-6-methyl-6,7- dihydro-5H-pyrrol[1,2- a]imidazole-3-sulfonamide | A2 | B33 | 407.1 |
| 73 | 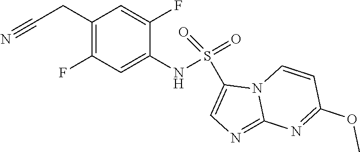 [SMILES: COc1ccn2c(S(=O)(=O)Nc3cc(F)c(CC#N)cc3F)cnc2n1] | N-[4-(cyanomethyl)-2,5- difluoro-phenyl]-7- methoxy-imidazo[1,2- a]pyrimidine-3-sulfonamide | A11 | B18 | 380.1 |
| 74 | 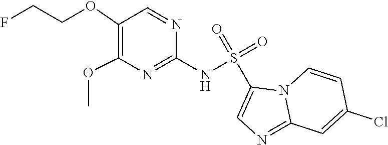 [SMILES: COc1nc(NS(=O)(=O)c2cnc3cc(Cl)ccn23)ncc1OCCF] | 7-chloro-N-[5-(2- fluoroethoxy)-4-methoxy- pyrimidin-2-yl]imidazo[1,2- a]pyridine-3-sulfonamide | A22 | B6 | 402.2 |
| 75 | 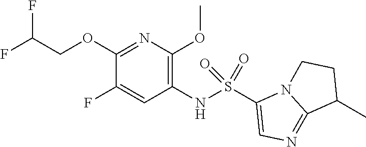 [SMILES: COc1nc(OCC(F)F)c(F)cc1NS(=O)(=O)c1cnc2n1CCC2C] | N-[6-(2,2-difluoroethoxy)- 5-fluoro-2-methoxy-3- pyridyl]-7-methyl-6,7- dihydro-5H-pyrrol[1,2- a]imidazole-3-sulfonamide | A2 | B34 | 407.1 |
| 76 | 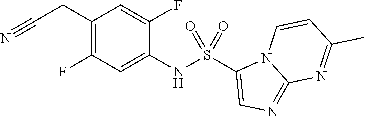 [SMILES: Cc1ccn2c(S(=O)(=O)Nc3cc(F)c(CC#N)cc3F)cnc2n1] | N-[4-(cyanomethyl)-2,5- difluoro-phenyl]-7-methyl- imidazo[1,2-a]pyrimidine-3- sulfonamide | A11 | B21 | 364.1 |
| 77 | 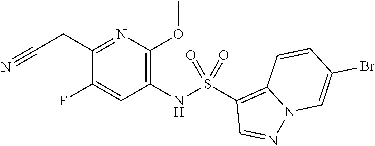 [SMILES: COc1nc(CC#N)c(F)cc1NS(=O)(=O)c1cnn2cc(Br)ccc12] | 6-bromo-N-[6- (cyanomethyl)-5-fluoro-2- methoxy-3- pyridyl]pyrazolo[1,5- a]pyridine-3-sulfonamide | A3 | B35 | 442.1 |
| 78 | 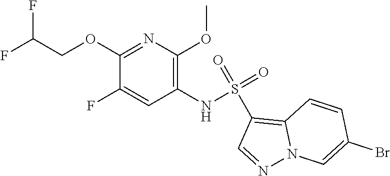 [SMILES: COc1nc(OCC(F)F)c(F)cc1NS(=O)(=O)c1cnn2cc(Br)ccc12] | 6-bromo-N-[6-(2,2- difluoroethoxy)-5-fluoro-2- methoxy-3- pyridyl]pyrazolo[1,5- a]pyridine-3-sulfonamide | A2 | B35 | 483.2 |
| 79 | 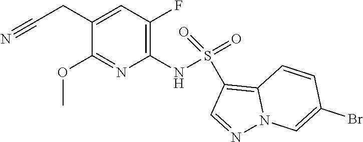 [SMILES: COc1nc(NS(=O)(=O)c2cnn3cc(Br)ccc23)c(F)cc1CC#N] | 6-bromo-N-[5- (cyanomethyl)-3-fluoro-6- methoxy-2- pyridyl]pyrazolo[1,5- a]pyridine-3-sulfonamide | A8 | B35 | 440.2 |
| 80 | 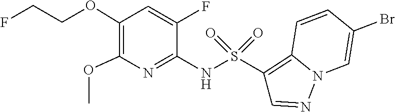 [SMILES: COc1nc(NS(=O)(=O)c2cnn3cc(Br)ccc23)c(F)cc1OCCF] | 6-bromo-N-[3-fluoro-5-(2- fluoroethoxy)-6-methoxy-2- pyridyl]pyrazolo[1,5- a]pyridine-3-sulfonamide | A10 | B35 | 465.1 |
| 81 | 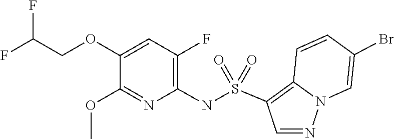 | 6-bromo-N-[5-(2,2- difluoroethoxy)-3-fluoro-6- methoxy-2- pyridyl]pyrazolo[1,5- a]pyridine-3-sulfonamide | A12 | B35 | 483.0 |
| 82 | 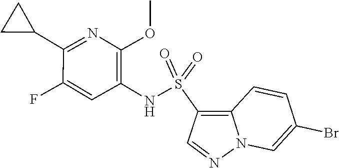 [SMILES: COc1nc(C2CC2)c(F)cc1NS(=O)(=O)c1cnn2cc(Br)ccc12] | 6-bromo-N-(6-cyclopropyl- 5-fluoro-2-methoxy-3- pyridyl)pyrazolo[1,5- a]pyridine-3-sulfonamide | A9 | B35 | 443.1 |
| 83 | 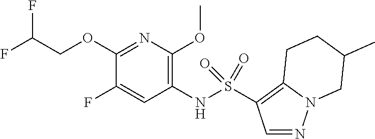 [SMILES: COc1nc(OCC(F)F)c(F)cc1NS(=O)(=O)c1cnn2c1CCC(C)C2] | N-[6-(2,2-difluoroethoxy)- 5-fluoro-2-methoxy-3- pyridyl]-6-methyl-4,5,6,7- tetrahydropyrazolo[1,5- a]pyridine-3-sulfonamide | A2 | B36 | 421.3 |
| 84 | 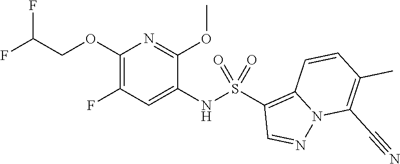 [SMILES: COc1nc(OCC(F)F)c(F)cc1NS(=O)(=O)c1cnn2c(C#N)c(C)ccc12] | 7-cyano-N-[6-(2,2- difluoroethoxy)-5-fluoro-2- methoxy-3-pyridyl]-6- methyl-pyrazolo[1,5- a]pyridine-3-sulfonamide | A2 | B37 | 442.2 |
| 85 | 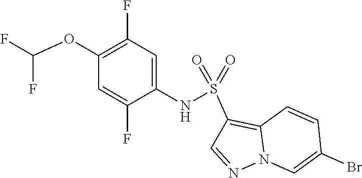 [SMILES: O=S(=O)(Nc1cc(F)c(OC(F)F)cc1F)c1cnn2cc(Br)ccc12] | 6-bromo-N-[4- (difluoromethoxy)-2,5- difluoro- phenyl]pyrazolo[1,5- a]pyridine-3-sulfonamide | A19 | B35 | 456.1 |
| 86 | 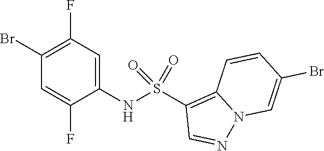 [SMILES: O=S(=O)(Nc1cc(F)c(Br)cc1F)c1cnn2cc(Br)ccc12] | 6-bromo-N-(4-bromo-2,5- difluoro- phenyl)pyrazolo[1,5- a]pyridine-3-sulfonamide | A18 | B35 | 468.0 |
| 87 | 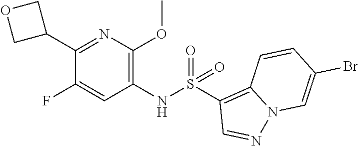 [SMILES: COc1nc(C2COC2)c(F)cc1NS(=O)(=O)c1cnn2cc(Br)ccc12] | 6-bromo-N-[5-fluoro-2- methoxy-6-(oxetan-3-yl)-3- pyridyl]pyrazolo[1,5- a]pyridine-3-sulfonamide | A20 | B35 | 459.1 |
| 88 | 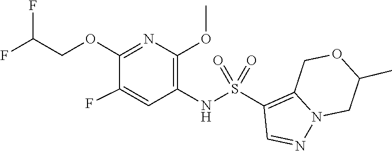 [SMILES: COc1nc(OCC(F)F)c(F)cc1NS(=O)(=O)c1cnn2c1COC(C)C2] | N-[6-(2,2-difluoroethoxy)- 5-fluoro-2-methoxy-3- pyridyl]-6-methyl-6,7- dihydro-4H-pyrazolo[5,1- c][1,4]oxazine-3- sulfonamide | A2 | B39 | 423.1 |
| 89 | 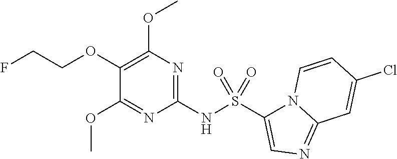 [SMILES: COc1nc(NS(=O)(=O)c2cnc3cc(Cl)ccn23)nc(OC)c1OCCF] | 7-chloro-N-[5-(2- fluoroethoxy)-4,6- dimethoxy-pyrimidin-2- yl]imidazo[1,2-a]pyridine- 3-sulfonamide | A24 | B6 | 432.1 |
| 90 | 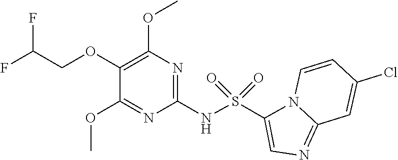 [SMILES: COc1nc(NS(=O)(=O)c2cnc3cc(Cl)ccn23)nc(OC)c1OCC(F)F] | 7-chloro-N-[5-(2,2- difluoroethoxy)-4,6- dimethoxy-pyrimidin-2- yl]imidazo[1,2-a]pyridine- 3-sulfonamide | A23 | B6 | 450.1 |
| 91 | 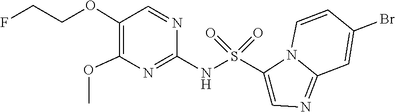 [SMILES: COc1nc(NS(=O)(=O)c2cnc3cc(Br)ccn23)ncc1OCCF] | 7-bromo-N-[5-(2- fluoroethoxy)-4-methoxy- pyrimidin-2- yl]imidazo[1,2-a]pyridine- 3-sulfonamide | A22 | B2 | 448.2 |
| 92 | 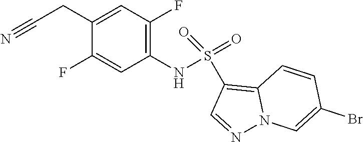 [SMILES: N#CCc1cc(F)c(NS(=O)(=O)c2cnn3cc(Br)ccc23)cc1F] | 6-bromo-N-[4- (cyanomethyl)-2,5- difluoro- phenyl]pyrazolo[1,5- a]pyridine-3-sulfonamide | A11 | B35 | 427.0 [M − H] |

### Example 93: N-[4-(cyanomethyl)-5-fluoro-2-methoxy-phenyl]-7-cyclopropyl-imidazo[1,2-a]pyridine-3-sulfonamide

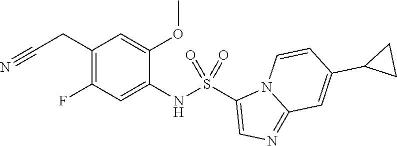 [SMILES: COc1cc(CC#N)c(F)cc1NS(=O)(=O)c1cnc2cc(C3CC3)ccn12]

5 A solution of 7-bromo-N-[4-(cyanomethyl)-5-fluoro-2-methoxy-phenyl]imidazo[1,2-a]pyridine-3-sulfonamide (60 mg, 0.137 mmol), cyclopropylboronic acid (15 mg, 0.178 mmol), PdCl2 (dppf)-CH2Cl2 (11 mg, 0.014 mmol) and cesium carbonate (133 mg, 0.410 mmol) in 1,4-dioxane (2.8 mL). The reaction mixture was stirred at 90° C. over night. The reaction mixture was poured into water and extracted with ethyl acetate. The combined organic layers were washed with brine, dried over sodium sulfate, filtered and concentrated in vacuo. The crude material was purified by flash on silica gel using a gradient heptane/ethyl acetate 0-100% to afford N-[4-(cyanomethyl)-5-fluoro-2-methoxy-phenyl]-7-cyclopropyl-imidazo[1,2-a]pyridine-3-sulfonamide (2.8 mg, 5%) as off-white solid. MS (ESI): m/z=401.2 [M+H]⁺

The following examples 90-92 were prepared in analogy to example 89 by Pd-mediated Suzuki cross-coupling reaction between bromo-imidazo sulfonamide and cyclopropylboronic acid.

| | | | MS (ESI): m/z, |
|---|---|---|---|
| Ex. | Structure | Name | [M + H]⁺ |
| 94 | 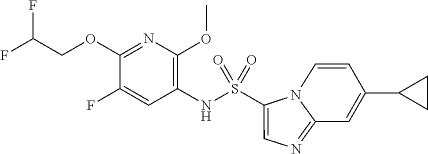 [SMILES: COc1nc(OCC(F)F)c(F)cc1NS(=O)(=O)c1cnc2cc(C3CC3)ccn12] | 7-cyclopropyl-N-[6-(2,2- difluoroethoxy)-5-fluoro-2-methoxy-3- pyridyl]imidazo[1,2-a]pyridine-3- sulfonamide | 443.2 |
| 95 | 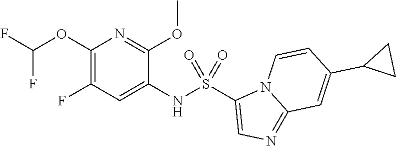 [SMILES: COc1nc(OC(F)F)c(F)cc1NS(=O)(=O)c1cnc2cc(C3CC3)ccn12] | 7-cyclopropyl-N-[6-(difluoromethoxy)- 5-fluoro-2-methoxy-3- pyridyl]imidazo[1,2-a]pyridine-3- sulfonamide | 429.2 |
| 96 | 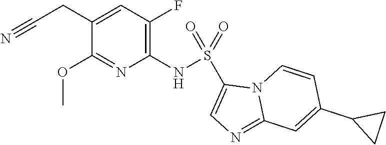 [SMILES: COc1nc(NS(=O)(=O)c2cnc3cc(C4CC4)ccn23)c(F)cc1CC#N] | N-[5-(cyanomethyl)-3-fluoro-6- methoxy-2-pyridyl]-7-cyclopropyl- imidazo[1,2-a]pyridine-3-sulfonamide | 402.2 |

### Example 97:7-cyclopropyl-N-[5-(difluoromethoxy)-3-fluoro-6-methoxy-2-pyridyl]imidazo[1,2-a]pyrimidine-3-sulfonamide

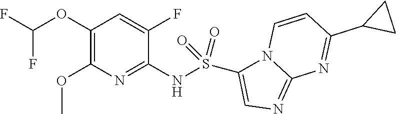 [SMILES: COc1nc(NS(=O)(=O)c2cnc3nc(C4CC4)ccn23)c(F)cc1OC(F)F]

A solution of [5-(difluoromethoxy)-3-fluoro-6-methoxy-2-pyridyl]amine (30 mg, 0.144 mmol) in N,N-dimethylformamide (500 μL) was cooled to 0° C. Sodium hydride (6.92 mg, 0.173 mmol) was added and the reaction mixture was stirred at 0° C. for 10 min. 7-cyclopropylimidazo[1,2-a]pyrimidine-3-sulfonyl chloride (40.86 mg, 0.159 mmol) was added. The reaction mixture was allowed to warm to room temperature and stirred for 1.5 h. The resulting dark purple mixture was quenched with water, poured into brine and extracted twice with ethyl acetate. The organic layers were dried over sodium sulfate, filtered and concentrated in vacuo. The crude material was purified by flash on silica gel using a gradient heptane/ethyl acetate 0-60% to afford 7-cyclopropyl-N-[5-(difluoromethoxy)-3-fluoro-6-methoxy-2-pyridyl]imidazo[1,2-a]pyrimidine-3-sulfonamide (3.3 mg, 5%) as light brown solid. MS (ESI): m/z=430.1 [M+H]⁺

The following examples 94 and 95 were prepared in analogy to example 93 by coupling the indicated sulfonylchloride intermediates B and amine intermediates A.

| | | | | | MS (ESI): |
|---|---|---|---|---|---|
| | | | | | m/z, |
| Ex. | Structure | Name | Int. A | Int. B | [M + H]⁺ |
| 98 | 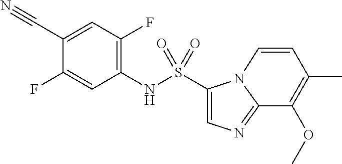 [SMILES: COc1c(C)ccn2c(S(=O)(=O)Nc3cc(F)c(C#N)cc3F)cnc12] | N-(4-cyano-2,5-difluoro- phenyl)-8-methoxy-7- methyl-imidazo[1,2- a]pyridine-3-sulfonamide | A21 | B4 | 379.1 |
| 99 | 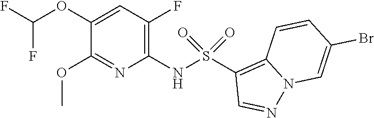 [SMILES: COc1nc(NS(=O)(=O)c2cnn3cc(Br)ccc23)c(F)cc1OC(F)F] | 6-bromo-N-[5- (difluoromethoxy)-3-fluoro- 6-methoxy-2- pyridyl]pyrazolo[1,5- a]pyridine-3-sulfonamide | A4 | B35 | 469.0 |

### Example 100: N-[6-(2,2-difluoroethoxy)-5-fluoro-2-methoxy-3-pyridyl]-7-(oxetan-3-yl)imidazo[1,2-a]pyridine-3-sulfonamide

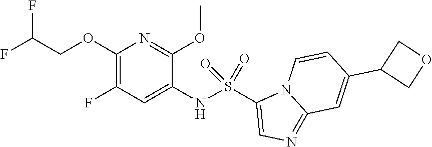 [SMILES: COc1nc(OCC(F)F)c(F)cc1NS(=O)(=O)c1cnc2cc(C3COC3)ccn12]

To an 15 mL vial equipped with a stir bar was added 7-bromo-N-[6-(2,2-difluoroethoxy)-5-fluoro-2-methoxy-3-pyridyl]imidazo[1,2-a]pyridine-3-sulfonamide (30.0 mg, 0.060 mmol), 3-bromooxetane (11.1 mg, 0.080 mmol), Ir[dF(CF3)ppy]2 (dtbpy)(PF6) (0.7 mg), NiCl2·dtbbpy (0.12 mg), tris (trimethylsilyl) silane (0.02 mL, 0.060 mmol), sodium carbonate (13.21 mg, 0.120 mmol) in dimethoxyethane (1 mL). The vial was sealed and placed under nitrogen. The reaction was stirred and irradiated with a 34 W blue LED lamp (7 cm away), with cooling fan to keep the reaction temperature at 25° C. After 14 h the reaction mixture was filtered and concentrated under reduced pressure. The residue was purified by pre-HPLC (condition: water (0.225% FA)-ACN, column: Phenomenex Gemini-NX C18 75*30 mm*3 μm) and freeze-dried to give N-[6-(2,2-difluoroethoxy)-5-fluoro-2-methoxy-3-pyridyl]-7-(oxetan-3-yl)imidazo[1,2-a]pyridine-3-sulfonamide (14 mg, 47%) as orange solid. MS (ESI): m/z=458.8 [M+H]⁺

### Example 101: N-[4-(cyanomethyl)-5-fluoro-2-methoxy-phenyl]-7-(oxetan-3-yl)imidazo[1,2-a]pyridine-3-sulfonamide

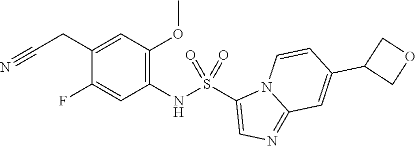 [SMILES: COc1cc(CC#N)c(F)cc1NS(=O)(=O)c1cnc2cc(C3COC3)ccn12]

The corresponding volume of the stock solution of Ir [dF (CF3) ppy] 2 (dtbpy) (PF6) (255.41 μg, 2.277E-04 mmol) in dichloromethane was added to the reaction vial and the solvent was evaporated. Then, sodium carbonate (4.83 mg, 0.046 mmol), 7-bromo-N-[4-(cyanomethyl)-5-fluoro-2-methoxy-phenyl]imidazo[1,2-a]pyridine-3-sulfonamide (10 mg, 0.023 mmol), 3-bromooxetane (4.05 mg, 2.46 μL, 0.030 mmol), tris (trimethylsilyl) silane (7.59 mg, 9.41 μL, 0.031 mmol) and ethylene glycol dimethyl ether (0.367 mL) were added. The reaction mixture was degassed by bubbling argon through for 5 min. Subsequently, the corresponding volume of the stock solution of 4,4″-di-tert-butyl-2,2″-dipyridyl (30.55 μg, 1.138E-04 mmol) and nickel (II) chloride ethylene glycol dimethyl (25.01 μg, 1.138E-04 mmol) in dimethoxyethane was added after sonication of the pastel green suspension. Finally, the mixture was again degassed by bubbling argon through for 10 min. The mixture was placed in the photoreactor and irradiated with 450 nm for 1 h (stirring at 900 rpm, 100% LED power). LCMS showed full conversion. The mixture was then filtered and concentrated in vacuo. The crude orange product was purified by flash chromatography on silica gel using a gradient heptane/ethyl acetate 0-100% to afford N-[4-(cyanomethyl)-5-fluoro-2-methoxy-phenyl]-7-(oxetan-3-yl)imidazo[1,2-a]pyridine-3-sulfonamide (3 mg, 28%) as off-white solid. MS (ESI): m/z-417.1 [M+H]^{+Example} 102: N-[5-(2,2-difluoroethyl)-3-fluoro-6-methoxy-2-pyridyl]-7-(oxetan-3-yl)imidazo[1,2-a]pyridine-3-sulfonamide

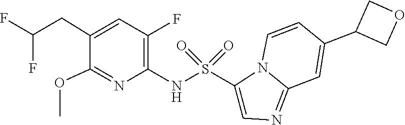 [SMILES: COc1nc(NS(=O)(=O)c2cnc3cc(C4COC4)ccn23)c(F)cc1CC(F)F]

The corresponding volume of the stock solution of Ir [dF (CF3) ppy] 2 (dtbpy) (PF6) (771.67 μg, 0.000688 mmol) in dichloromethane was added to the reaction vial and the solvent was evaporated. Then, sodium carbonate (14.58 mg, 0.138 mmol), 7-bromo-N-[5-(2,2-difluoroethyl)-3-fluoro-6-methoxy-2-pyridyl]imidazo[1,2-a]pyridine-3-sulfonamide (32 mg, 0.069 mmol), 3-bromooxetane (12.25 mg, 7.42 μL, 0.089 mmol), tris (trimethylsilyl) silane (22.92 mg, 28.43 μL, 0.092 mmol) and ethylene glycol dimethyl ether (1.11 mL) was added. The reaction mixture was degassed with bubbling argon through for 5 min. Subsequently, the corresponding volume of the stock solution of 4,4″-di-tert-butyl-2,2″-dipyridyl (92.3 μg, 3.439E-04 mmol) and nickel (II) chloride ethylene glycol dimethyl (75.56 μg, 3.439E-04 mmol) in dimethoxyethane was added after sonication of the pastel green suspension. Finally, the mixture was again degassed by bubbling argon through for 10 min. The mixture was placed in the photoreactor and irradiated with 450 nm for 15 min (stirring at 900 rpm, 100% LED power). LCMS showed product and desbromo side product. The mixture was filtered and evaporated. The residue was purified by flash chromatography using a gradient heptane/ethyl acetate 0-100% followed by C18 flash chromatography RediSepRf Gold column (C18, 5.5 g, Acetonitrile in water 10% to 100%) to afford N-[5-(2,2-difluoroethyl)-3-fluoro-6-methoxy-2-pyridyl]-7-(oxetan-3-yl)imidazo[1,2-a]pyridine-3-sulfonamide (4 mg, 11%) as off-white solid. MS (ESI): m/z=443.3 [M+H]⁺

### Example 103: N-[6-(2,2-difluoroethoxy)-5-fluoro-2-methoxy-3-pyridyl]-7-(oxetan-3-yl)imidazo[1,2-a]pyrimidine-3-sulfonamide

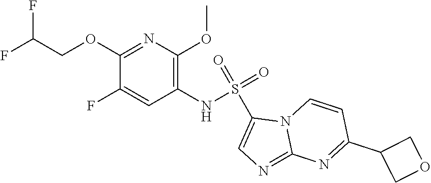 [SMILES: COc1nc(OCC(F)F)c(F)cc1NS(=O)(=O)c1cnc2nc(C3COC3)ccn12]

The reaction was performed on 10 mg scale (4 times). In a vial, Ir [dF (CF3) ppy] 2 (dtbpy) (PF6) (902.68 μg, 8.046E-04 mmol), sodium carbonate (17.06 mg, 0.161 mmol), 7-bromo-N-[6-(2,2-difluoroethoxy)-5-fluoro-2-methoxy-3-pyridyl]imidazo[1,2-a]pyrimidine-3-sulfonamide (38.8 mg, 0.080 mmol), 3-bromooxetane (14.33 mg, 8.68 μL, 0.105 mmol) and tris (trimethylsilyl) silane (26.81 mg, 33.26 μL, 0.108 mmol) were added. Ethylene glycol dimethyl ether (1.3 mL) was added and the mixture was degassed for 5 min. Then, nickel (II) chloride ethylene glycol dimethyl ether complex (88.39 μg, 4.023E-04 mmol) and 4,4″-di-tert-butyl-2 2″-dipyridyl (107.98 μg, 4.023E-04 mmol) (as stock solution in dimethoxyethane) were added. The mixture was again degassed for 10 min. The reaction mixture was irradiated with 450 nm in a photoreactor (100% LED power, 900 rpm stirring) for 15 h. The reaction mixture was filtered and concentrated in vacuo. The orange residue was purified by flash chromatography using a gradient heptane/ethyl acetate 0-100% to afford N-[6-(2,2-difluoroethoxy)-5-fluoro-2-methoxy-3-pyridyl]-7-(oxetan-3-yl)imidazo[1,2-a]pyrimidine-3-sulfonamide (combined 4 reactions: 4 mg, 8%) as yellow solid. MS (ESI): m/z=460.2 [M+H]⁺

### Example 104: N-[6-(2,2-difluoroethoxy)-5-fluoro-2-methoxy-3-pyridyl]-6-(oxetan-3-yl)pyrazolo[1,5-a]pyridine-3-sulfonamide

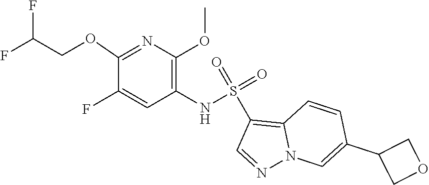 [SMILES: COc1nc(OCC(F)F)c(F)cc1NS(=O)(=O)c1cnn2cc(C3COC3)ccc12]

In a vial, Ir [dF (CF3) ppy] 2 (dtbpy) (PF6) (2.1 mg, 0.002 mmol), sodium carbonate (39.64 mg, 0.374 mmol), 6-bromo-N-[6-(2,2-difluoroethoxy)-5-fluoro-2-methoxy-3-pyridyl]pyrazolo[1,5-a]pyridine-3-sulfonamide (90 mg, 0.187 mmol), 3-bromooxetane (51.24 mg, 31.05 μL, 0.374 mmol) and tris (trimethylsilyl) silane (62.32 mg, 77.31 μL, 0.251 mmol) were added. Ethylene glycol dimethyl ether (3.02 mL) was added and the mixture was degassed for 5 min. Then, nickel (II) chloride ethylene glycol dimethyl ether complex (205.46 μg, 9.351E-04 mmol) and 4,4″-di-tert-butyl-2 2″-dipyridyl (250.97 μg, 9.351E-04 mmol) (as stock solution in dimethoxyethane) were added. The mixture was again degassed for 10 min. The reaction mixture was irradiated with 450 nm in a photoreactor (100% LED power, 900 rpm stirring) for 14 h. The reaction mixture was concentrated, dissolved in dichloromethane, filtered and purified by flash chromatography on silica gel using a gradient heptane/ethyl acetate 0-100% followed by purification by prep-HPLC and SFC to afford N-[6-(2,2-difluoroethoxy)-5-fluoro-2-methoxy-3-pyridyl]-6-(oxetan-3-yl)pyrazolo[1,5-a]pyridine-3-sulfonamide (4.3 mg, 4.5%) as light yellow solid. MS (ESI): m/z=459.2 [M+H]⁺

### Example 105:7-bromo-N-[6-(2,2-difluoroethoxy)-5-fluoro-2-methoxy-3-pyridyl]imidazo[1,2-a]pyrimidine-3-sulfonamide

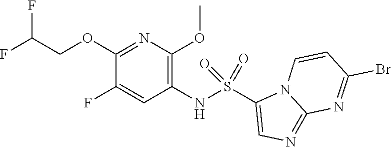 [SMILES: COc1nc(OCC(F)F)c(F)cc1NS(=O)(=O)c1cnc2nc(Br)ccn12]

To an orange-brown suspension of 7-chloro-N-[6-(2,2-difluoroethoxy)-5-fluoro-2-methoxy-3-pyridyl]imidazo[1,2-a]pyrimidine-3-sulfonamide (62.9 mg, 0.144 mmol) in propionitrile (2.37 g, 3. mL, 43.1 mmol) bromotrimethylsilane (329.94 mg, 284.44 μL, 2.16 mmol) was added. The reaction mixture was heated to 50° C. After 5 h the reaction mixture was slowly added to a stirring solution of NaOH 2N/ice and extracted with ethyl acetate. The combined organic phases were dried over sodium sulfate, filtered and concentrated in vacuo. The residue was purified by flash chromatography on silica gel using a gradient heptane/ethyl acetate 0-100% to afford 7-bromo-N-[6-(2,2-difluoroethoxy)-5-fluoro-2-methoxy-3-pyridyl]imidazo[1,2-a]pyrimidine-3-sulfonamide (38.8 mg, 56%) as off-white solid. MS (ESI): m/z=484.1 [M+H]⁺

### Example 106: N-[6-(2,2-difluoroethoxy)-5-fluoro-2-methoxy-3-pyridyl]-7-(1-hydroxy-1-methyl-ethyl)imidazo[1,2-a]pyridine-3-sulfonamide

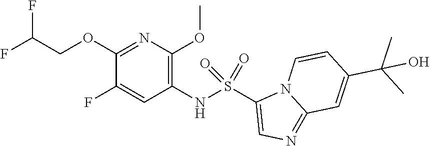 [SMILES: COc1nc(OCC(F)F)c(F)cc1NS(=O)(=O)c1cnc2cc(C(C)(C)O)ccn12]

A solution of 3-[[6-(2,2-difluoroethoxy)-5-fluoro-2-methoxy-3-pyridyl] sulfamoyl]imidazo[1,2-a]pyridine-7-carboxylic acid methyl ester (10 mg, 0.022 mmol) in tetrahydrofuran (100 μL) was cooled to 0° C. 3 M methylmagnesium bromide in diethyl ether (37.47 mg, 36.2 μL, 0.109 mmol) was added dropwise. The reaction mixture was stirred at 0° C. for 1 h before it was quenched with sat. NH4Cl and extracted twice with ethyl acetate. The organic layers were dried over sodium sulfate, filtered and concentrated in vacuo. The crude material was purified by flash chromatography on silica gel using a gradient heptane/ethyl acetate 0-100% to afford N-[6-(2,2-difluoroethoxy)-5-fluoro-2-methoxy-3-pyridyl]-7-(1-hydroxy-1-methyl-ethyl)imidazo[1,2-a]pyridine-3-sulfonamide (5.6 mg, 56%) as a colorless solid. MS (ESI): m/z=461.1 [M+H]^{+Example} 107: N-[6-(2,2-difluoroethoxy)-5-fluoro-2-methoxy-3-pyridyl]-7-(3-hydroxyoxetan-3-yl)imidazo[1,2-a]pyridine-3-sulfonamide

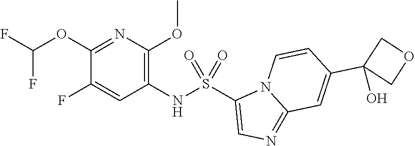 [SMILES: COc1nc(OC(F)F)c(F)cc1NS(=O)(=O)c1cnc2cc(C3(O)COC3)ccn12]

To a stirred solution of 7-bromo-N-[6-(2,2-difluoroethoxy)-5-fluoro-2-methoxy-3-pyridyl]imidazo[1,2-a]pyridine-3-sulfonamide (43 mg, 0.089 mmol) in tetrahydrofuran (860 μL) was added 1.3 M Turbo Grignard (196.09 mg, 206.2 μL, 0.268 mmol) at 0° C. The reaction mixture was stirred at 0° C. for 1 h before oxetan-3-one (19.32 mg, 15.7 μL, 0.268 mmol) was added. The reaction mixture was stirred at room temperature for 90 min before it was quenched with sat. NH4Cl solution and extracted twice with ethyl acetate. The organic layers were dried over sodium sulfate, filtered and concentrated in vacuo. The residue was purified by prep-HPLC to afford N-[6-(2,2-difluoroethoxy)-5-fluoro-2-methoxy-3-pyridyl]-7-(3-hydroxyoxetan-3-yl)imidazo[1,2-a]pyridine-3-sulfonamide (3 mg, 6%) as a colorless solid. MS (ESI): m/z=475.1 [M+H]⁺

### Example 108 and Example 109: (7R)—N-[6-(2,2-difluoroethoxy)-5-fluoro-2-methoxy-3-pyridyl]-7-methyl-5,6,7,8-tetrahydroimidazo[1,2-a]pyridine-3-sulfonamide and (7S)—N-[6-(2,2-difluoroethoxy)-5-fluoro-2-methoxy-3-pyridyl]-7-methyl-5,6,7,8-tetrahydroimidazo[1,2-a]pyridine-3-sulfonamide

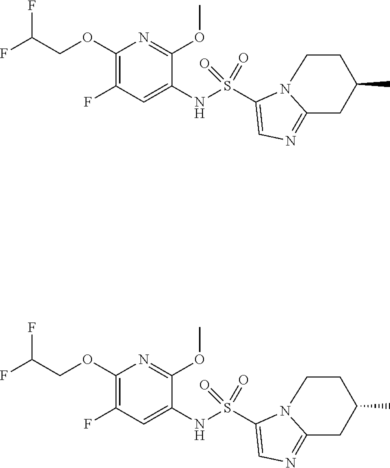 [SMILES: COc1nc(OCC(F)F)c(F)cc1NS(=O)(=O)c1cnc2n1CC[C@@H](C)C2 ; COc1nc(OCC(F)F)c(F)cc1NS(=O)(=O)c1cnc2n1CC[C@H](C)C2]

Example 23 was subjected to chiral separation using SFC (Column: Chiralpak IC-3 50×4.6 mm I.D., 3 μm Mobile phase: Phase A for CO2, and Phase B for IPA (0.05% DEA); Gradient elution: IPA (0.05% DEA) in CO2 from 5% to 40%, Flow rate: 3 mL/min; Detector: PDA,Column Temp: 35C; Back Pressure: 100Bar″) (running time-2.109) to yield both enantiomers as a off-white solid.

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula I 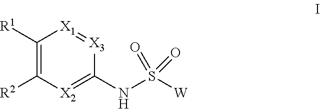
wherein,
R¹ is alkyl, cyano, cyanoalkyl, cyclopropyl, halo, haloalkoxy, haloalkyl or oxetanyl;
R² is alkoxy, H or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy, H, or halo;
R¹² is H or halo;
R¹³ is alkoxy, H, halo or haloalkoxy;
W is selected from Ring Systems A, B, C, D, or E 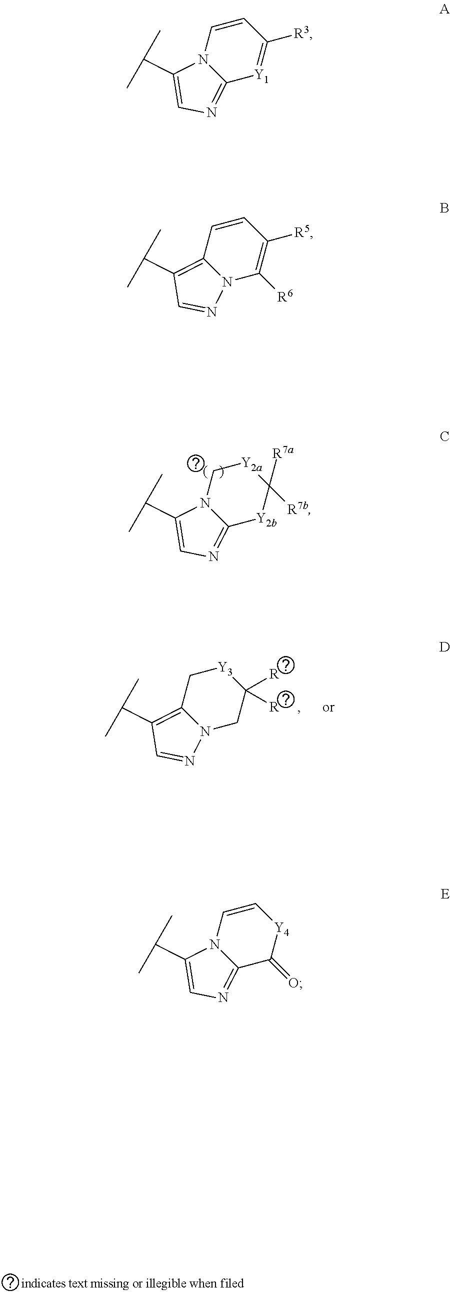
Y¹ is CR⁴ or N;
R³ is alkoxy, alkyl, cyano, cyclopropyl, H, halo, haloalkoxy, haloalkyl, hydroxyalkyl, hydroxyoxetanyl, or oxetanyl;
R⁴ alkoxy, alkyl, cyano, H, or halo;
R⁵ is alkyl, halo, haloalkyl, cyclopropyl or oxetanyl;
R⁶ is H, cyano, alkyl, alkoxy or halo;
Y₂ₐ is CR^{8a} or O and Y_{2b} is CR^{8b} or O, wherein only one of Y₂ₐ and Y_{2b} can be O;
n is 0 or 1;
R^{7a} and R^{7b} are independently selected from H, alkyl, alkoxy, or haloalkyl;
R^{8a} and R^{9b} are independently selected from H or alkyl;
Y³ is O or CH₂;
R^{9a} and R^{9b} are independently selected from H, alkyl, alkoxy, or haloalkyl;
Y⁴ is NR 10;
R¹⁰ is alkyl or haloalkyl;
and pharmaceutically acceptable salts thereof.

2. A compound according to claim 1, wherein Y₂ₐ is CH₂ and Y_{2b} is CR⁸b or O, wherein R^{8b} is H or alkyl.

3. A compound according to claim 1, wherein R^{9a} or R^{9b} are alkyl and the other is H.

4. A compound according to claim 1, wherein R¹ is alkyl, cyanoalkyl, cyclopropyl, halo, haloalkoxy or haloalkyl.

5. A compound according to claim 1, wherein R² is alkoxy or halo.

6. A compound according to claim 1, wherein R¹¹ is alkoxy or H.

7. A compound according to claim 1, wherein R¹² is H.

8. A compound according to claim 1, wherein R¹³ is alkoxy.

9. A compound according to claim 1, wherein W is selected from Ring Systems A, B or C.

10. A compound according to claim 1, wherein R³ is alkoxy, alkyl, cyclopropyl, halo, haloalkoxy, haloalkyl, hydroxyalkyl or oxetanyl.

11. A compound according to claim 1, wherein R⁴ is alkoxy, cyano, H or halo.

12. A compound according to claim 1, wherein R⁵ is halo and R⁶ is H.

13. A compound according to claim 1, wherein when R^{7a}, R^{7b}, R^{8a} and R^{9b} are all H then X₁ is N and n is 1.

14. A compound according to claim 1, wherein
R¹ is alkyl, cyano, cyanoalkyl, cyclopropyl, halo, haloalkoxy, haloalkyl or oxetanyl;
R² is alkoxy, H or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³,
R¹¹ is alkoxy, H, or halo;
R¹² is H or halo;
R¹³ is alkoxy, H, halo or haloalkoxy;
W is selected from Ring Systems A, B, C, D, or E 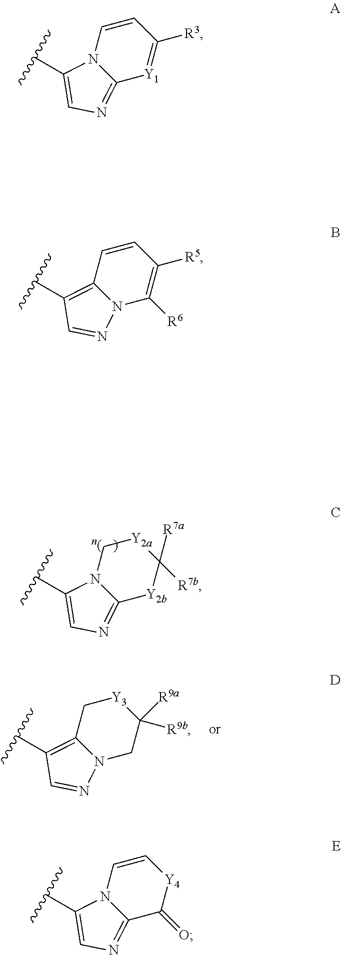
Y₁ is CR⁴ or N;
R³ is alkoxy, alkyl, cyano, cyclopropyl, H, halo, haloalkoxy, haloalkyl, hydroxyalkyl, hydroxyoxetanyl, or oxetanyl;
R⁴ alkoxy, alkyl, cyano, H, or halo;
R⁵ alkyl, halo or oxetanyl;
R⁶ cyano or H;
Y₂ₐ is CH₂ and Y_{2b} is CR^{8b} or O, wherein R^{8b} is H or alkyl;
nis 0 or 1;
R^{7a} and R^{7b} are independently selected from H, alkyl, alkoxy, or haloalkyl;
Y³ is O or CH₂;
R^{9a} or R^{9b} are alkyl and the other is H;
Y⁴ is NR 10;
R¹⁰ is alkyl;
wherein when R^{7a}, R^{7b}, R^{8a} and R^{8b} are all H then X₁ is N and n is 1;
and pharmaceutically acceptable salts thereof.

15. A compound according to claim 1, wherein
R¹ is alkyl, cyanoalkyl, cyclopropyl, halo, haloalkoxy, or haloalkyl;
R² is alkoxy or halo;
X₁ is N, X₂ is CR¹² and X₃ is CR¹³, or
X₁ is CR¹¹, X₂ is CR¹², and X₃ is CR¹³, or
X₁ is CR¹¹, X₂ is N, and X₃ is N or CR¹³;
R¹¹ is alkoxy or H;
R¹² is H;
R¹³ is alkoxy, halo or haloalkoxy;
W is selected from Ring Systems A, B, or C 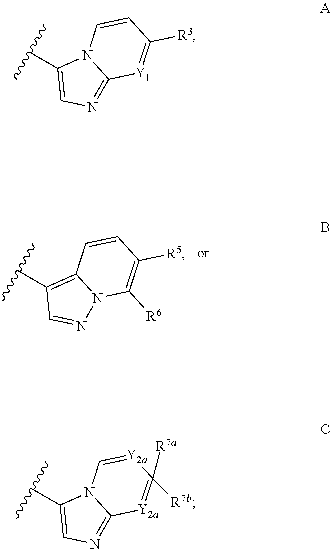
Y₁ is CR⁴ or N;
R³ is alkoxy, alkyl, cyclopropyl, halo, haloalkoxy, haloalkyl, hydroxyalkyl, or oxetanyl;
R⁴ is alkoxy, cyano, H, or halo;
R⁵ is halo;
R⁶ is H;
Y₂ₐ is CH₂ and Y_{2b} is CR^{8b} or O, wherein R^{8b} is H or alkyl;
n is 1;
R^{7a} and R^{7b} are independently selected from H, alkyl, or haloalkyl;
wherein when R^{7a}, R^{7b}, R^{8a} and R^{8b} are all H then X₁ is N and n is 1;
and pharmaceutically acceptable salts thereof.

16. A compound according to claim 1, selected from
7-bromo-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyridine-3-sulfonamide;
7-bromo-N-[4-(cyanomethyl)-5-fluoro-2-methoxy-phenyl]imidazo[1,2-a]pyridine-3-sulfonamide;
N-[6-(2,2-difluoroethoxy)-5-fluoro-2-methoxy-3-pyridyl]-8-methoxy-7-methyl-imidazo[1,2-a]pyridine-3-sulfonamide;
N-[6-(2,2-difluoroethoxy)-5-fluoro-2-methoxy-3-pyridyl]-7-(difluoromethoxy)imidazo[1,2-a]pyridine-3-sulfonamide;
7-chloro-N-[6-(2,2-difluoroethoxy)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-fluoro-imidazo[1,2-a]pyridine-3-sulfonamide;
7-bromo-N-[6-(difluoromethoxy)-5-fluoro-2-methoxy-3-pyridyl]imidazo[1,2-a]pyridine-3-sulfonamide;
7-chloro-N-[6-(3,3-difluoropropyl)-5-fluoro-2-methoxy-3-pyridyl]imidazo[1,2-a]pyridine-3-sulfonamide;
7-bromo-N-[5-(2,2-difluoroethyl)-3-fluoro-6-methoxy-2-pyridyl]imidazo[1,2-a]pyridine-3-sulfonamide;
7-chloro-N-[6-(2,2-difluoroethyl)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-methoxy-imidazo[1,2-a]pyridine-3-sulfonamide;
N-[6-(difluoromethoxy)-5-fluoro-2-methoxy-3-pyridyl]-7-methyl-imidazo[1,2-a]pyridine-3-sulfonamide;
N-[6-(2,2-difluoroethoxy)-5-fluoro-2-methoxy-3-pyridyl]-7-(difluoromethyl)imidazo[1,2-a]pyridine-3-sulfonamide;
N-[6-(2,2-difluoroethoxy)-5-fluoro-2-methoxy-3-pyridyl]-7-methoxy-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-ethyl-imidazo[1,2-a]pyridine-3-sulfonamide;
N-[4-(cyanomethyl)-5-fluoro-2-methoxy-phenyl]-7-cyclopropyl-imidazo[1,2-a]pyrimidine-3-sulfonamide;
N-[6-(difluoromethoxy)-5-fluoro-2-methoxy-3-pyridyl]-8-methoxy-7-methyl-imidazo[1,2-a]pyridine-3-sulfonamide;
N-[4-(cyanomethyl)-5-fluoro-2-methoxy-phenyl]-7-methyl-imidazo[1,2-a]pyridine-3-sulfonamide;
7-bromo-N-[5-(cyanomethyl)-3-fluoro-6-methoxy-2-pyridyl]imidazo[1,2-a]pyridine-3-sulfonamide;
7-chloro-N-[6-(cyanomethyl)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-5,6,7,8-tetrahydroimidazo[1,2-a]pyridine-3-sulfonamide;
7-chloro-N-[5-(2,2-difluoroethyl)-3-fluoro-6-methoxy-2-pyridyl]imidazo[1,2-a]pyridine-3-sulfonamide;
7-chloro-N-(6-cyclopropyl-5-fluoro-2-methoxy-3-pyridyl)imidazo[1,2-a]pyridine-3-sulfonamide;
7-bromo-N-[5-(difluoromethoxy)-3-fluoro-6-methoxy-2-pyridyl]imidazo[1,2-a]pyridine-3-sulfonamide;
8-cyano-N-[6-(2,2-difluoroethoxy)-5-fluoro-2-methoxy-3-pyridyl]-7-methyl-imidazo[1,2-a]pyridine-3-sulfonamide;
7-cyclopropyl-N-[6-(difluoromethoxy)-5-fluoro-2-methoxy-3-pyridyl]imidazo[1,2-a]pyrimidine-3-sulfonamide;
N-[6-(difluoromethoxy)-5-fluoro-2-methoxy-3-pyridyl]-7-methoxy-imidazo[1,2-a]pyridine-3-sulfonamide;
N-[6-(2,2-difluoroethoxy)-5-fluoro-2-methoxy-3-pyridyl]-7,8-dimethyl-5,6,7,8-tetrahydroimidazo[1,2-a]pyridine-3-sulfonamide;
N-[6-(2,2-difluoroethoxy)-5-fluoro-2-methoxy-3-pyridyl]-7-(trifluoromethyl)imidazo[1,2-a]pyridine-3-sulfonamide;
N-[4-(cyanomethyl)-2,5-difluoro-phenyl]-8-methoxy-7-methyl-imidazo[1,2-a]pyridine-3-sulfonamide;
8-cyano-7-cyclopropyl-N-[6-(2,2-difluoroethoxy)-5-fluoro-2-methoxy-3-pyridyl]imidazo[1,2-a]pyridine-3-sulfonamide;
7-bromo-N-[4-(cyanomethyl)-2,5-difluoro-phenyl]imidazo[1,2-a]pyridine-3-sulfonamide;
N-[5-(cyanomethyl)-3-fluoro-6-methoxy-2-pyridyl]-7-methoxy-imidazo[1,2-a]pyridine-3-sulfonamide;
7-cyclopropyl-N-[5-(2,2-difluoroethyl)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-7-methoxy-imidazo[1,2-a]pyrimidine-3-sulfonamide;
N-[2,6-bis(difluoromethoxy)-5-fluoro-3-pyridyl]-7-chloro-imidazo[1,2-a]pyridine-3-sulfonamide;
7-chloro-N-[6-(difluoromethoxy)-5-fluoro-2-methoxy-3-pyridyl]imidazo[1,2-a]pyridine-3-sulfonamide;
7-chloro-N-[5-(difluoromethoxy)-3-fluoro-6-methoxy-2-pyridyl]imidazo[1,2-a]pyridine-3-sulfonamide;
N-[6-(2,2-difluoroethoxy)-5-fluoro-2-methoxy-3-pyridyl]-7-(trifluoromethyl)-5,6,7,8-tetrahydroimidazo[1,2-a]pyridine-3-sulfonamide;
7-chloro-N-[6-(2,2-difluoroethoxy)-5-fluoro-2-methoxy-3-pyridyl]imidazo[1,2-a]pyrimidine-3-sulfonamide;
8-cyano-N-[6-(difluoromethoxy)-5-fluoro-2-methoxy-3-pyridyl]-7-methyl-imidazo[1,2-a]pyridine-3-sulfonamide;
7-chloro-N-(5-cyclopropyl-3-fluoro-6-methoxy-2-pyridyl)imidazo[1,2-a]pyridine-3-sulfonamide;
N-[6-(2,2-difluoroethoxy)-5-fluoro-2-methoxy-3-pyridyl]-7,7-dimethyl-6,8-dihydro-5H-imidazo[1,2-a]pyridine-3-sulfonamide;
7-bromo-N-(5-bromo-4,6-dimethoxy-pyrimidin-2-yl)imidazo[1,2-a]pyridine-3-sulfonamide;
7-bromo-N-[3-fluoro-5-(2-fluoroethoxy)-6-methoxy-2-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyrimidine-3-sulfonamide;
7-cyclopropyl-N-[5-(2,2-difluoroethoxy)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-8-methyl-5,6,7,8-tetrahydroimidazo[1,2-a]pyridine-3-sulfonamide;
7-chloro-N-[5-(2,2-difluoroethoxy)-3-fluoro-6-methoxy-2-pyridyl]imidazo[1,2-a]pyridine-3-sulfonamide;
7-chloro-8-cyano-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-6,7-dihydro-5H-imidazo[2,1-b][1,3]oxazine-3-sulfonamide;
N-(4,6-dimethoxy-5-methyl-pyrimidin-2-yl)-7-methyl-imidazo[1,2-a]pyridine-3-sulfonamide;
N-[4-(cyanomethyl)-2,5-difluoro-phenyl]-7-methoxy-imidazo[1,2-a]pyridine-3-sulfonamide;
N-[2,6-bis(difluoromethoxy)-5-fluoro-3-pyridyl]-7-cyclopropyl-imidazo[1,2-a]pyrimidine-3-sulfonamide;
N-(4-bromo-2,5-difluoro-phenyl)-8-methoxy-7-methyl-imidazo[1,2-a]pyridine-3-sulfonamide;
N-[6-(2,2-difluoroethoxy)-5-fluoro-2-methoxy-3-pyridyl]-8-keto-7-methyl-imidazo[1,2-a]pyrazine-3-sulfonamide;
7-cyano-N-[6-(2,2-difluoroethoxy)-5-fluoro-2-methoxy-3-pyridyl]imidazo[1,2-a]pyridine-3-sulfonamide;
8-cyano-N-[6-(2,2-difluoroethoxy)-5-fluoro-2-methoxy-3-pyridyl]-7-methoxy-imidazo[1,2-a]pyridine-3-sulfonamide;
7-chloro-N-[4-(cyanomethyl)-2,5-difluoro-phenyl]-8-methoxy-imidazo[1,2-a]pyridine-3-sulfonamide;
N-[6-(2,2-difluoroethoxy)-5-fluoro-2-methoxy-3-pyridyl]-7-methoxy-5,6,7,8-tetrahydroimidazo[1,2-a]pyridine-3-sulfonamide;
N-[6-(2,2-difluoroethoxy)-5-fluoro-2-methoxy-3-pyridyl]-5,6,7,8-tetrahydroimidazo[1,2-a]pyridine-3-sulfonamide;
7-chloro-N-(4-(cyanomethyl)-2,5-difluorophenyl)imidazo[1,2-a]pyridine-3-sulfonamide;
8-cyano-N-[6-(2,2-difluoroethoxy)-5-fluoro-2-methoxy-3-pyridyl]-7-(difluoromethoxy)imidazo[1,2-a]pyridine-3-sulfonamide;
N-[2,6-bis(difluoromethoxy)-5-fluoro-3-pyridyl]-8-cyano-7-methyl-imidazo[1,2-a]pyridine-3-sulfonamide;
8-cyano-N-[4-(cyanomethyl)-2,5-difluoro-phenyl]-7-methyl-imidazo[1,2-a]pyridine-3-sulfonamide;
7-chloro-N-[4-(cyanomethyl)-2,5-difluoro-phenyl]-8-fluoro-imidazo[1,2-a]pyridine-3-sulfonamide;
8-cyano-N-[6-(2,2-difluoroethoxy)-5-fluoro-2-methoxy-3-pyridyl]imidazo[1,2-a]pyridine-3-sulfonamide;
7-chloro-N-[5-fluoro-2-methoxy-6-(oxetan-3-yl)-3-pyridyl]imidazo[1,2-a]pyridine-3-sulfonamide;
N-[6-(2,2-difluoroethoxy)-5-fluoro-2-methoxy-3-pyridyl]-8-methyl-imidazo[1,2-a]pyridine-3-sulfonamide;
N-[6-(2,2-difluoroethoxy)-5-fluoro-2-methoxy-3-pyridyl]-6-methyl-6,7-dihydro-5H-pyrrol [1,2-a]imidazole-3-sulfonamide;
N-[4-(cyanomethyl)-2,5-difluoro-phenyl]-7-methoxy-imidazo[1,2-a]pyrimidine-3-sulfonamide;
7-chloro-N-[5-(2-fluoroethoxy)-4-methoxy-pyrimidin-2-yl]imidazo[1,2-a]pyridine-3-sulfonamide;
N-[6-(2,2-difluoroethoxy)-5-fluoro-2-methoxy-3-pyridyl]-7-methyl-6,7-dihydro-5H-pyrrol [1,2-a]imidazole-3-sulfonamide;
N-[4-(cyanomethyl)-2,5-difluoro-phenyl]-7-methyl-imidazo[1,2-a]pyrimidine-3-sulfonamide;
6-bromo-N-[6-(cyanomethyl)-5-fluoro-2-methoxy-3-pyridyl]pyrazolo[1,5-a]pyridine-3-sulfonamide;
6-bromo-N-[6-(2,2-difluoroethoxy)-5-fluoro-2-methoxy-3-pyridyl]pyrazolo[1,5-a]pyridine-3-sulfonamide;
6-bromo-N-[5-(cyanomethyl)-3-fluoro-6-methoxy-2-pyridyl]pyrazolo[1,5-a]pyridine-3-sulfonamide;
6-bromo-N-[3-fluoro-5-(2-fluoroethoxy)-6-methoxy-2-pyridyl]pyrazolo[1,5-a]pyridine-3-sulfonamide;
6-bromo-N-[5-(2,2-difluoroethoxy)-3-fluoro-6-methoxy-2-pyridyl]pyrazolo[1,5-a]pyridine-3-sulfonamide;
6-bromo-N-(6-cyclopropyl-5-fluoro-2-methoxy-3-pyridyl)pyrazolo[1,5-a]pyridine-3-sulfonamide;
N-[6-(2,2-difluoroethoxy)-5-fluoro-2-methoxy-3-pyridyl]-6-methyl-4,5,6,7-tetrahydropyrazolo[1,5-a]pyridine-3-sulfonamide;
7-cyano-N-[6-(2,2-difluoroethoxy)-5-fluoro-2-methoxy-3-pyridyl]-6-methyl-pyrazolo[1,5-a]pyridine-3-sulfonamide;
6-bromo-N-[4-(difluoromethoxy)-2,5-difluoro-phenyl]pyrazolo[1,5-a]pyridine-3-sulfonamide;
6-bromo-N-(4-bromo-2,5-difluoro-phenyl)pyrazolo[1,5-a]pyridine-3-sulfonamide;
6-bromo-N-[5-fluoro-2-methoxy-6-(oxetan-3-yl)-3-pyridyl]pyrazolo[1,5-a]pyridine-3-sulfonamide;
N-[6-(2,2-difluoroethoxy)-5-fluoro-2-methoxy-3-pyridyl]-6-methyl-6,7-dihydro-4H-pyrazolo[5,1-c][1,4]oxazine-3-sulfonamide;
N-[4-(cyanomethyl)-5-fluoro-2-methoxy-phenyl]-7-cyclopropyl-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyridine-3-sulfonamide;
7-cyclopropyl-N-[6-(difluoromethoxy)-5-fluoro-2-methoxy-3-pyridyl]imidazo[1,2-a]pyridine-3-sulfonamide;
N-[5-(cyanomethyl)-3-fluoro-6-methoxy-2-pyridyl]-7-cyclopropyl-imidazo[1,2-a]pyridine-3-sulfonamide;
7-cyclopropyl-N-[5-(difluoromethoxy)-3-fluoro-6-methoxy-2-pyridyl]imidazo[1,2-a]pyrimidine-3-sulfonamide;
N-(4-cyano-2,5-difluoro-phenyl)-8-methoxy-7-methyl-imidazo[1,2-a]pyridine-3-sulfonamide;
6-bromo-N-[5-(difluoromethoxy)-3-fluoro-6-methoxy-2-pyridyl]pyrazolo[1,5-a]pyridine-3-sulfonamide;
N-[6-(2,2-difluoroethoxy)-5-fluoro-2-methoxy-3-pyridyl]-7-(oxetan-3-yl)imidazo[1,2-a]pyridine-3-sulfonamide;
N-[4-(cyanomethyl)-5-fluoro-2-methoxy-phenyl]-7-(oxetan-3-yl)imidazo[1,2-a]pyridine-3-sulfonamide;
N-[5-(2,2-difluoroethyl)-3-fluoro-6-methoxy-2-pyridyl]-7-(oxetan-3-yl)imidazo[1,2-a]pyridine-3-sulfonamide;
N-[6-(2,2-difluoroethoxy)-5-fluoro-2-methoxy-3-pyridyl]-7-(oxetan-3-yl)imidazo[1,2-a]pyrimidine-3-sulfonamide;
N-[6-(2,2-difluoroethoxy)-5-fluoro-2-methoxy-3-pyridyl]-6-(oxetan-3-yl)pyrazolo[1,5-a]pyridine-3-sulfonamide;
7-bromo-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-(1-hydroxy-1-methyl-ethyl)imidazo[1,2-a]pyridine-3-sulfonamide;
N-[6-(2,2-difluoroethoxy)-5-fluoro-2-methoxy-3-pyridyl]-7-(3-hydroxyoxetan-3-yl)imidazo[1,2-a]pyridine-3-sulfonamide;
and pharmaceutically acceptable salts thereof.

17. A compound according to claim 1, selected from
7-bromo-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyridine-3-sulfonamide;
7-bromo-N-[4-(cyanomethyl)-5-fluoro-2-methoxy-phenyl]imidazo[1,2-a]pyridine-3-sulfonamide;
N-[6-(2,2-difluoroethoxy)-5-fluoro-2-methoxy-3-pyridyl]-8-methoxy-7-methyl-imidazo[1,2-a]pyridine-3-sulfonamide;
N-[6-(2,2-difluoroethoxy)-5-fluoro-2-methoxy-3-pyridyl]-7-(difluoromethoxy)imidazo[1,2-a]pyridine-3-sulfonamide;
7-chloro-N-[6-(2,2-difluoroethoxy)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-fluoro-imidazo[1,2-a]pyridine-3-sulfonamide;
7-bromo-N-[6-(difluoromethoxy)-5-fluoro-2-methoxy-3-pyridyl]imidazo[1,2-a]pyridine-3-sulfonamide;
7-chloro-N-[6-(3,3-difluoropropyl)-5-fluoro-2-methoxy-3-pyridyl]imidazo[1,2-a]pyridine-3-sulfonamide;
7-bromo-N-[5-(2,2-difluoroethyl)-3-fluoro-6-methoxy-2-pyridyl]imidazo[1,2-a]pyridine-3-sulfonamide;
7-chloro-N-[6-(2,2-difluoroethyl)-5-fluoro-2-methoxy-3-pyridyl]imidazo[1,2-a]pyridine-3-sulfonamide;
7-chloro-N-[6-(2,2-difluoroethoxy)-5-fluoro-2-methoxy-3-pyridyl]-8-methoxy-imidazo[1,2-a]pyridine-3-sulfonamide;
N-[6-(difluoromethoxy)-5-fluoro-2-methoxy-3-pyridyl]-7-methyl-imidazo[1,2-a]pyridine-3-sulfonamide;
N-[6-(2,2-difluoroethoxy)-5-fluoro-2-methoxy-3-pyridyl]-7-(difluoromethyl)imidazo[1,2-a]pyridine-3-sulfonamide;
N-[6-(2,2-difluoroethoxy)-5-fluoro-2-methoxy-3-pyridyl]-7-methoxy-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-ethyl-imidazo[1,2-a]pyridine-3-sulfonamide;
N-[4-(cyanomethyl)-5-fluoro-2-methoxy-phenyl]-7-cyclopropyl-imidazo[1,2-a]pyrimidine-3-sulfonamide;
N-[6-(difluoromethoxy)-5-fluoro-2-methoxy-3-pyridyl]-8-methoxy-7-methyl-imidazo[1,2-a]pyridine-3-sulfonamide;
N-[4-(cyanomethyl)-5-fluoro-2-methoxy-phenyl]-7-methyl-imidazo[1,2-a]pyridine-3-sulfonamide;
7-bromo-N-[5-(cyanomethyl)-3-fluoro-6-methoxy-2-pyridyl]imidazo[1,2-a]pyridine-3-sulfonamide;
7-chloro-N-[6-(cyanomethyl)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-5,6,7,8-tetrahydroimidazo[1,2-a]pyridine-3-sulfonamide;
7-chloro-N-[5-(2,2-difluoroethyl)-3-fluoro-6-methoxy-2-pyridyl]imidazo[1,2-a]pyridine-3-sulfonamide;
7-chloro-N-(6-cyclopropyl-5-fluoro-2-methoxy-3-pyridyl)imidazo[1,2-a]pyridine-3-sulfonamide;
7-bromo-N-[5-(difluoromethoxy)-3-fluoro-6-methoxy-2-pyridyl]imidazo[1,2-a]pyridine-3-sulfonamide;
8-cyano-N-[6-(2,2-difluoroethoxy)-5-fluoro-2-methoxy-3-pyridyl]-7-methyl-imidazo[1,2-a]pyridine-3-sulfonamide;
7-cyclopropyl-N-[6-(difluoromethoxy)-5-fluoro-2-methoxy-3-pyridyl]imidazo[1,2-a]pyrimidine-3-sulfonamide;
N-[6-(difluoromethoxy)-5-fluoro-2-methoxy-3-pyridyl]-7-methoxy-imidazo[1,2-a]pyridine-3-sulfonamide;
N-[6-(2,2-difluoroethoxy)-5-fluoro-2-methoxy-3-pyridyl]-7,8-dimethyl-5,6,7,8-tetrahydroimidazo[1,2-a]pyridine-3-sulfonamide;
N-[6-(2,2-difluoroethoxy)-5-fluoro-2-methoxy-3-pyridyl]-7-(trifluoromethyl)imidazo[1,2-a]pyridine-3-sulfonamide;
N-[4-(cyanomethyl)-2,5-difluoro-phenyl]-8-methoxy-7-methyl-imidazo[1,2-a]pyridine-3-sulfonamide;
8-cyano-7-cyclopropyl-N-[6-(2,2-difluoroethoxy)-5-fluoro-2-methoxy-3-pyridyl]imidazo[1,2-a]pyridine-3-sulfonamide;
7-bromo-N-[4-(cyanomethyl)-2,5-difluoro-phenyl]imidazo[1,2-a]pyridine-3-sulfonamide;
N-[5-(cyanomethyl)-3-fluoro-6-methoxy-2-pyridyl]-7-methoxy-imidazo[1,2-a]pyridine-3-sulfonamide;
7-cyclopropyl-N-[5-(2,2-difluoroethyl)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-7-methoxy-imidazo[1,2-a]pyrimidine-3-sulfonamide;
N-[2,6-bis(difluoromethoxy)-5-fluoro-3-pyridyl]-7-chloro-imidazo[1,2-a]pyridine-3-sulfonamide;
7-chloro-N-[6-(difluoromethoxy)-5-fluoro-2-methoxy-3-pyridyl]imidazo[1,2-a]pyridine-3-sulfonamide;
7-chloro-N-[5-(difluoromethoxy)-3-fluoro-6-methoxy-2-pyridyl]imidazo[1,2-a]pyridine-3-sulfonamide;
N-[6-(2,2-difluoroethoxy)-5-fluoro-2-methoxy-3-pyridyl]-7-(trifluoromethyl)-5,6,7,8-tetrahydroimidazo[1,2-a]pyridine-3-sulfonamide;
7-chloro-N-[6-(2,2-difluoroethoxy)-5-fluoro-2-methoxy-3-pyridyl]imidazo[1,2-a]pyrimidine-3-sulfonamide;
8-cyano-N-[6-(difluoromethoxy)-5-fluoro-2-methoxy-3-pyridyl]-7-methyl-imidazo[1,2-a]pyridine-3-sulfonamide;
7-chloro-N-(5-cyclopropyl-3-fluoro-6-methoxy-2-pyridyl)imidazo[1,2-a]pyridine-3-sulfonamide;
N-[6-(2,2-difluoroethoxy)-5-fluoro-2-methoxy-3-pyridyl]-7,7-dimethyl-6,8-dihydro-5H-imidazo[1,2-a]pyridine-3-sulfonamide;
7-bromo-N-(5-bromo-4,6-dimethoxy-pyrimidin-2-yl)imidazo[1,2-a]pyridine-3-sulfonamide;
7-bromo-N-[3-fluoro-5-(2-fluoroethoxy)-6-methoxy-2-pyridyl]imidazo[1,2-a]pyridine-3-sulfonamide;
N-[6-(2,2-difluoroethoxy)-5-fluoro-2-methoxy-3-pyridyl]-7-methyl-imidazo[1,2-a]pyrimidine-3-sulfonamide;
7-cyclopropyl-N-[5-(2,2-difluoroethoxy)-3-fluoro-6-methoxy-2-pyridyl]imidazo[1,2-a]pyrimidine-3-sulfonamide;
N-[6-(2,2-difluoroethoxy)-5-fluoro-2-methoxy-3-pyridyl]-8-methyl-5,6,7,8-tetrahydroimidazo[1,2-a]pyridine-3-sulfonamide;
7-chloro-N-[5-(2,2-difluoroethoxy)-3-fluoro-6-methoxy-2-pyridyl]imidazo[1,2-a]pyridine-3-sulfonamide;
7-chloro-8-cyano-N-[6-(2,2-difluoroethoxy)-5-fluoro-2-methoxy-3-pyridyl]imidazo[1,2-a]pyridine-3-sulfonamide;
N-[6-(2,2-difluoroethoxy)-5-fluoro-2-methoxy-3-pyridyl]-7-methyl-6,7-dihydro-5H-imidazo[2,1-b][1,3]oxazine-3-sulfonamide;
N-(4,6-dimethoxy-5-methyl-pyrimidin-2-yl)-7-methyl-imidazo[1,2-a]pyridine-3-sulfonamide;
6-bromo-N-(6-cyclopropyl-5-fluoro-2-methoxy-3-pyridyl)pyrazolo[1,5-a]pyridine-3-sulfonamide;
N-[4-(cyanomethyl)-5-fluoro-2-methoxy-phenyl]-7-cyclopropyl-imidazo[1,2-a]pyridine-3-sulfonamide;
7-cyclopropyl-N-[6-(2,2-difluoroethoxy)-5-fluoro-2-methoxy-3-pyridyl]imidazo[1,2-a]pyridine-3-sulfonamide;
7-cyclopropyl-N-[6-(difluoromethoxy)-5-fluoro-2-methoxy-3-pyridyl]imidazo[1,2-a]pyridine-3-sulfonamide;
N-[5-(cyanomethyl)-3-fluoro-6-methoxy-2-pyridyl]-7-cyclopropyl-imidazo[1,2-a]pyridine-3-sulfonamide;
7-cyclopropyl-N-[5-(difluoromethoxy)-3-fluoro-6-methoxy-2-pyridyl]imidazo[1,2-a]pyrimidine-3-sulfonamide;
6-bromo-N-[5-(difluoromethoxy)-3-fluoro-6-methoxy-2-pyridyl]pyrazolo[1,5-a]pyridine-3-sulfonamide;
N-[6-(2,2-difluoroethoxy)-5-fluoro-2-methoxy-3-pyridyl]-7-(oxetan-3-yl)imidazo[1,2-a]pyridine-3-sulfonamide;
7-bromo-N-[6-(2,2-difluoroethoxy)-5-fluoro-2-methoxy-3-pyridyl]imidazo[1,2-a]pyrimidine-3-sulfonamide;
N-[6-(2,2-difluoroethoxy)-5-fluoro-2-methoxy-3-pyridyl]-7-(1-hydroxy-1-methyl-ethyl)imidazo[1,2-a]pyridine-3-sulfonamide;
and pharmaceutical salts thereof.

18. A process of preparing a compound of claim 1 comprising reacting of a compound of formula V with a compound of formula VI in the presence of a base to provide a compound of formula I,

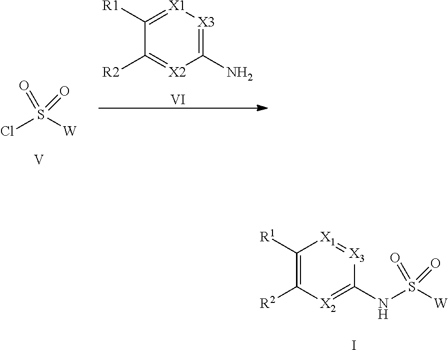

19-20. (canceled)

21. A pharmaceutical composition comprising a compound according to claim 1 and a therapeutically inert carrier.

22-26. (canceled)

27. A method for the treatment or prophylaxis of conditions resulting from direct damage to myelin sheaths, demyelinating disorders, CNS disorders associated with myelin loss, and inflammation in the CNS, which method comprises administering an effective amount of a compound according to claim 1 to a patient in need thereof.

28. A method for the treatment or prophylaxis of multiple sclerosis, which method comprises administering an effective amount of a compound according to claim 1 to a patient in need thereof.

29-30. (canceled)

31. The method of claim 27, wherein the disease or condition is central pontine and extra-pontine myelinolysis, carbon monoxide poisoning, nutritional deficiency, or virus-induced demyelination.

32. The method of claim 27, wherein the disease or condition is multiple sclerosis, acute and multiphasic disseminated encephalomyelitis, neuromyelitis optica spectrum disorders, or leukodystrophies.

33. The method of claim 27, wherein the disease or condition is Alzheimer's disease, schizophrenia, Parkinson's disease, Huntington's disease, Amyotrophic lateral sclerosis, or Ischemia due to stroke.

34. The method of claim 27, wherein the disease or condition is inflammation related to encephalitis, primary angiitis, meningitis or obesity.
